(19) United States
(12) Patent Application Publication
(10) Pub. No.: US 2010/0240597 A1
(43) Pub. Date: Sep. 23, 2010

(54) METHODS OF DELIVERY OF MOLECULES TO CELLS USING A RICIN SUBUNIT AND COMPOSITIONS RELATING TO SAME

(75) Inventors: Carole L. Cramer, Jonesboro, AR (US); Maureen C. Dolan, Jonesboro, AR (US); Michael J. Reidy, Rockville, MD (US)

Correspondence Address: JOE D. CALHOUN, P.O. BOX 251504, LITTLE ROCK, AR, 72225, US

(73) Assignee: ARKANSAS STATE UNIVERSITY, State University, AR (US)

(21) Appl. No.: 12/664,342

(22) PCT Filed: Jun. 12, 2008
(86) PCT No.: PCT/US2008/066790

§ 371 Date: Dec. 11, 2009

Related U.S. Application Data
(60) Provisional application No. 60/944,193, filed on Jun. 15, 2007.

(51) Int. Cl. A61K 38/12; C07K 14/415; C12P 21/06; C12N 5/02; C12N 5/10

(52) U.S. Cl. 514/21.2; 530/370; 435/70.1; 435/375; 435/419

(57) ABSTRACT

A method of preparing molecules of interest for delivery to eukaryotic cells is shown, wherein a ricin B chain subunit not having a ribosome inactivating subunit and retaining lectin activity is modified by modifying the first cysteine residue to be absent or substituted with an amino acid other than cysteine, or removing a protease sensitive site at the N-terminal of the subunit, or adding an endoplasmic reticulum retrieval signal, and operatively associating the subunit with a molecule of interest. Methods of operatively associating the subunit and molecule of interest include chemical conjugation at primary amines, conjugation with N-glycans of the subunit, a disulfide bond, and assembly of immunoglobulin domains. The invention provides for operatively associating multiple molecules of interest with a ricin B chain subunit, and delivery into targeted cells, cell components, and combination of cells.

This application claims priority to previously filed and co-pending application U.S. Ser. No. 60/944,193, filed Jun. 15, 2007, the contents of which and all references cited here are incorporated herein by reference in their entirety.

### BACKGROUND OF THE INVENTION

Expression of heterologous proteins in eukaryotic and prokaryotic cells is employed in many biotechnology settings for various purposes such as in expressing proteins which impact the cell itself, and in which the cell is a host for production of the protein. Such processes hold particular promise in production of industrial and pharmaceutically useful proteins. In many instances, obtaining adequate expression levels of the protein by the host or in the target cell can be challenging, as can targeting adequate amounts of the protein of interest to the cell to be impacted. For example, when delivering a pharmaceutically useful protein to an animal cell, it is necessary to provide adequate amounts to achieve the desired response, as well as deliver the protein to the target cell and in a manner that is therapeutically useful.

The castor bean plant (Ricinus communis) has developed a defense mechanism that employs production of the type II ribosome inactivating protein ricin. As is described in more detail below, ricin is toxic and is used by the plant to defend against attacking agents. The ricin toxin consists of the A-chain (RTA, an N-glycosidase that inactivates ribosomes) and the B-chain (RTB, a galactose/N-galactosamine specific lectin) that are joined via a single disulfide bond. The B-chain (RTB) triggers endocytosis by binding to surface glycoproteins and glycolipids on target cells, thereby delivering RTA to ribosomes in the cytoplasm through a multi-part pathway. A large portion of ricin toxin remains in the lysosomal compartments, and smaller portions accumulate in the endoplasmic reticulum. When ricin A and ricin B are in the endoplasmic reticulum, they dissociate and RTA is translocated to the cytoplasm. Ricin also has the capacity to delivery “across” as well as into a cell by cycling between endosomes, lysosomes and the cell surface.

Attempts have been made to determine if it is possible to employ ricin as a delivery agent for antigen or toxin delivery. Most efforts to date use the entire ricin protein, isolated from castor bean. In some cases, attempts have been made to specifically purify the RTB lectin subunit away from the RTA toxic subunit. Concerns arise with the potential that even after extensive steps to remove ricin A have been performed, toxins may remain. In one instance, recombinant RTB was expressed in hairy roots of plants and used to produced immune response to an associated protein in mice. (Medina-Bolivar et al.). There remains a need to find an efficient system to produce a toxin-free form of RTB to aid in the delivery of various molecules.

### SUMMARY OF THE INVENTION

The invention is directed to methods of preparing a molecule of interest for delivery to a eukaryotic cell where a recombinant ricin B chain subunit which does not have a ribosome inactivating subunit and retains lectin activity is modified and operatively associated with a molecule of interest. In an embodiment the modification provides for a different amino acid at the first cysteine residue of the subunit other than cysteine, in another embodiment provides for truncation at the N-terminal to remove the a protease sensitive site, and in another embodiment adds an endoplasmic reticulum retrieval signal. Other embodiments provide for means of operatively associating the molecule of interest through conjugation, covalent binding, protein-protein interactions and genetic fusion. Embodiments provide for conjugation of molecules of interest at the primary amines of the subunit, in another embodiment conjugation with N-linked glycans of the subunit, and in a further embodiment bonding of molecules of interest to the subunit through disulfide bonds. Still further embodiments provide for fusing the molecule of interest at the N-terminus of the subunit, and another embodiment provides for fusion at the C-terminus of the subunit. Use of immunoglobulin domains for association of the molecule of interest is provided in an embodiment, and association through a breakable disulfide bond provided in yet another. Expression levels in host cells of the subunit are at least about 0.1% total soluble protein in a further embodiment.

### DESCRIPTION OF THE DRAWINGS

FIG. 1A shows an example of a ricin B chain subunit encoding nucleotide (SEQ ID NO: 1), FIG. 1B shows an example of an amino acid sequence comprising a ricin B chain subunit (SEQ ID NO: 2) and FIG. 1C shows a comparison of SEQ ID NO: 2 and six other ricin B chain subunit amino acid sequences (SEQ ID NO: 3-8).

FIG. 2A is a graphic representation of engineering an immunoglobulin domain-based scaffold to connect a carrier to the payload. FIG. 2B is a graphic representation of ricin pathways.

FIG. 3 shows recombinant RTB and RTB:GFP gene construct maps.

FIG. 4 shows generation of RTB-specific antibodies in rabbit with a gel (A) of soluble/misfolded E. coli-derived RTB used as antigen in developing antibodies and a Western blot (B) of plant-derived RTB probed with rabbit anti-RTB.

FIG. 5 is a gel showing purification of recombinant RTB and RTB:GFP conjugation of fluorescein and biotin to rRTB.

FIG. 6 shows maps of plant-expressed RTB-carrier constructs.

FIG. 7 shows maps of E. coli-derived payload constructs.

FIG. 8 shows maps of plant-derived payload constructs.

FIG. 9 shows Western blots of carrier constructs.

FIG. 10 is a gel of purified RTB-containing fusion proteins.

FIG. 11 is a Western blot showing migration patterns of RTB:κLC under different reducing conditions.

FIG. 12 shows gels of purification of GST:Fd:TC and removal of the GST tag by thrombin digestion, and addition of Lumio Green® reagent prior to gel loading, photographed with and without UV light-box to show TC-mediated fluorescence.

FIG. 13 shows gels of plant-produced carrier and payload by anti-RTB (left) and anti-GFP (right) Western analysis after 15 minute exposure (top) and three hour exposure (bottom).

FIG. 14 is a graph summarizing analysis of κLC-Fd mediated interactions via asialofetuin assays and GFP ELISA.

FIG. 15 is a Western blot of plant-synthesized product purified based on RTB activity.

FIG. 16 is a gel showing analysis of RTB: κLC-Fd*:GFP interaction with anti-RTB Western (A) and silver-stained 10% SDS-PAGE of purified samples (B)

FIG. 17 shows a graphic depiction of processing of ricin by castor bean.

FIG. 18 shows maps of constructs.

FIG. 19 shows representation of five proteins producing breakdown produces that were sequenced (A); Coomassie stained members of sequence RTB-purified proteins (B); and results of N-terminal sequencing on indicated bands (C).

FIG. 20 shows a gel of C_{L}:link:RTB(tr) via anti-RTB Western (A) and Coomassie-strained membrane of RTB-purified C_{L}:link:RTB(tr).

FIG. 21 shows anti-RTB Western analysis comparing lactose-binding fractions generated from infiltration of five different constructs.

FIG. 22 shows summaries of C_{L}:RTB and three mutated constructs generated via site-directed mutagenesis (A); and an anti-RTB Western blot of lactose-binding fractions generated from point mutations (B).

FIG. 23 shows a map of a construct.

FIG. 24 shows maps of three constructs.

FIG. 25 depicts the process of preparation of the RTB:L:IL-12 pBC construct.

FIG. 26 is a Western blot of RTB:IL-12 plants and non-transgenic plants with anti-mIL.

FIG. 27 shows a gel with a comparison of RTB-12 fusions recovered by lactose affinity chromatography and probed with polyclonal mIL-12 antibody (A) or RTB antibody (B).

FIG. 28 is a gel with purified IL-12:RTB and equivalent fractions from non-transgenic control (NT) (A) and purified IL-12:RTB transferred to membrane for anti-RTB Western blotting analysis (B) or stained by Coomassie stain (C).

FIG. 29 shows four graphs, representing IL-12 bioactivity assay in mouse splenocytes. In (A) splenocytes from mice were cultured in indicated amounts of animal cell-derived mIL-12, (acdIL-12), IL-12:RTB purified from transgenic hairy roots or equivalent fractions from non-transgenic controls (NT). Supernatants were assayed for IFN-γ concentration by ELISA. In (B) Induction of IFN-γ in the presence or absence of IL-12 neutralizing antibody is shown. In (C) results of standard colorimetric cell proliferation assays are shown on splenocytes. PHA-preactivated splenocytes were cultured with indicated amounts of acdIL-12, IL-12:RTB or NT control. In (D) a comparison is shown of cell proliferation in splenocytes treated with IL-12 in the presence or absence of neutralizing antibodies.

FIG. 30(A) shows a schematic of testing IL-12:RTB in a MALT in vitro model. FIG. 30(B) graphs results of purification of IL-12:RTB or IL-12 from transgenic plants added to inserts containing a HT-29 monolayer, incubation to allow RTB to bind to the cell surface, culturing with splenocytes and collection of supernatant for IFN-γ ELISAs.

### DESCRIPTION OF EMBODIMENTS OF THE INVENTION

The inventors have developed improved methodologies for delivery of molecules of interest. Ricin toxin is known to effectively traverse mucosal surfaces and to partition between various pathways upon endocytosis, mainly through the interactions of the galactose/N-acetylgalactosamine-specific lectin B-subunit (RTB) with target cell proteins. Exploitation of ricin's endocytotic and sub-cellular movement characteristics was achieved through the use of recombinant RTB (rRTB). Recombinant RTB is expressed in a host cell, which can be an insect, animal, plant or yeast cell and in one preferred embodiment is a plant cell, and operatively associated with a molecule of interest. Expression levels in excess of 0.1% total soluble protein of RTB were achieved. Expression levels of 0.2% TSP, 0.3% TSP, 0.4% TSP, 0.5% TSP, 1% TSP, 5% TSP and up to 10% TSP can be achieved. The RTB may be recovered from the plant cell and conjugated or otherwise combined with a molecule of interest, through any variety of mechanisms available to one skilled in the art, or the molecule of interest may be operatively linked with the RTB in the host cell and then introduced to the target cell. The RTB and molecule of interest are then delivered to the target cell, where the RTB binds to the surface glycans of the target cell and triggers endocytosis. The RTB is able to deliver the operatively associated molecule to the target cell.

In preferred embodiments, the RTB includes the B-chain subunit, and not the RTA, and yet retains lectin activity. In an embodiment of the invention, the RTB is expressed such that it is modified, such that the amino acids comprising a protease sensitive site at the N-terminal of the subunit are modified to remove the site. In an embodiment, a protease sensitive site at the N-terminal is modified such that the activity of the site is eliminated. One embodiment provides the first six N-terminal amino acids A, D, V, C/S, M and D are not present. This is especially useful when attaching a molecule of interest at the N-terminal of the subunit, as it decreases cleavage and promotes stability of the product comprising the molecule of interest fused to the ricin B subunit. By referring to such truncated RTB proteins, it is intended that the removal of the amino acids may occur by any known biotechnology methods, or the protease sensitive site can also be removed by substituting different amino acids such that the activity of the site is eliminated. The nucleotide sequence encoding the RTB may be truncated, mutated or otherwise modified, or the amino acids cleaved by virtue of post-translational methods available to one skilled in the art. In an example, one can remove the nucleotide sequences that encode for the amino acids. This prevents cleaving of the RTB and stabilizes the fusion product. Yield increases from about 50% to 80% to about 90% to 100%.

In an additional embodiment, the amino acids at the N-terminus of the recombinant ricin B subunit are retained but the region is modified by substituting the first cysteine residue (CYS₄) of the ricin B chain subunit with another amino acid. This modification is preferred when the molecule of interest is associated as a genetic fusion to the C-terminus of the subunit or is added after the ricin B chain subunit is synthesize. Through this modification, it is possible to eliminate the site for sulfhydryl bonding which can cause bonding to other ricin B molecules or other proteins. In a separate embodiment, the first cysteine residue is left intact in order to specifically provide an unpaired cysteine on the ricin B subunit to permit formation of a disulfide bond between the ricin B chain subunit and the molecule of interest.

As noted, the RTB can be operatively associated with the molecule of interest by either various chemical interactions of the molecule of interest with the RTB after RTB is expressed in the host cell, or can be co-expressed in the host cell. Means of operatively associating the molecule of interest by a chemical interaction are described herein, and can include covalent binding via chemical conjugation and protein-protein interactions. If co-expressed, the fusion product can then be used for delivery to the target cell. The inventors have discovered it is possible to operatively associate the molecule of interest at either the C- or N-terminus of the RTB and retain lectin activity. In some cases, association of the molecule of interest specifically at the C-terminus or the N-terminus of RTB is preferred in order to retain full activity of the molecule of interest. Additionally, a linker can be placed between the molecule of interest and the ricin B chain subunit to provide adequate spacing for both the molecule of interest and the ricin B chain subunit to retain full activity or to provide a detection tag or cleavage potential. Preferred embodiments provided the molecule is associated with the C-terminus of the RTB, which they have discovered avoids the cleavage which can occur otherwise, and which would reduce yield of the useful fusion RTB-molecule of interest. However, by fusing the molecule of interest to the N-terminus of truncated RTB, the likelihood of this cleavage is greatly reduced.

Use of immunoglobulin domain assembling is also provided with the invention, where an immunoglobulin domain is fused to the ricin B subunit, and can assemble with the molecule of interest, if the molecule of interest also comprises a second immunoglobulin domain known to assemble with the first immunoglobulin domain. In this embodiment, the ricin B chain subunit could be used to deliver, for example, a fully assembled monoclonal antibody or an Fab domain (light chain assembled with the Fd portion of the heavy chain and retains its binding specificity) into a cell or cell compartment to activate or inhibit a specific process. In an additional embodiment, the second immunoglobulin domain may be fused to the molecule of interest and the first and second domains assemble to operatively associate the subunit and molecule of interest, as more fully described below.

Another embodiment provides that multiple molecules of interest, either multiple copies of the same molecule, or different molecules, may be operatively associated with a single RTB. Delivery of multiple molecules to the target cell provides considerable advantages. The inventors discovered the binding activity of the RTB is not adversely affected, and large or small molecules can be associated with the RTB and delivered to the target cell. The multiple copies can be provided either by expressing multiple copies of the molecule of interest with the RTB in the host cell, or using the methods described below to recover RTB from the host and operatively link multiple copies of the molecule of interest with the RTB.

Proper processing of the RTB is also achieved in a preferred embodiment in which a signal peptide sequence is expressed in the host cell with the RTB such that the RTB is preferentially expressed in the host cell endoplasmic reticulum. Native RTB is a glycoprotein and is typically synthesized in association with the endoplasmic reticulum. Any functional signal peptide will be useful in directing the RTB through the endomembrane system of the host cell so that proper glycosylation may occur. The signal peptide will be cleaved during synthesis so the RTB in its final form will not have the signal peptide product. As discussed below, it may be then chemically associated with the molecule of interest, or recovered with the molecule of interest already associated by expression of both in the host cell.

Further, by adding an endoplasmic reticulum retrieval sequence (KDEL, HDEL, SEKDEL, etc.) to be expressed as a fused protein with the RTB, it was discovered it is possible to carry the peptide sequence product through with the RTB and use endoplasmic reticulum retrieval sequence to direct more of the molecule of interest to the endoplasmic reticulum (ER) in the target cell. By using of the term endoplasmic reticulum retrieval sequence is intended to encompass the alternative term of an endoplasmic reticulum retention signal, as long as the sequence directs or redirects the protein to the endoplasmic reticulum. The endoplasmic reticulum retrieval sequence was able to re-direct location of the molecule of interest within the target cell. Under normal circumstances, most RTB, when delivered to the target cell, migrates to the lysosomes and about 10% or less is directed to the endoplasmic reticulum. By combining an endoplasmic reticulum retrieval sequence with the RTB, interaction with the KDEL receptor in the target cells occurred and moved the RTB and the associated molecule of interest to the endoplasmic reticulum. This can be useful in applications where delivery to the endoplasmic reticulum of the target cell is preferred, such as in the case of the vaccine applications, where delivery of antigen to the ER results in a greater T_{H}1 or cell-mediated immune response. Delivery to the ER may also be useful, for example, when delivering a molecule of interest comprising a therapeutic enzyme whose site of action resides within the ER. Selectively directing molecules to the ER, which is closely associated with the nucleus, may also be preferred when the molecule of interest comprises a DNA sequence, for example one used for gene therapy, or an RNA sequences, for example one used in RNAi strategies to inhibit an endogenous gene activity in the target cell.

Fusing the RTB with the targeting peptide for later direction to the endoplasmic reticulum of the target cell may not be preferred where instead it is desired to take advantage of the ability of RTB to be directed to the lysosome of the target cell for delivery of the molecule of interest. This is useful, for example, when delivering a molecule of interest for treatment of lysosomal diseases, such as the delivery of glucocerebrosidase to Gaucher Disease patients or iduronidase to Hurler's Syndrome patients. Delivery to the lysosome may also be the preferred route in the case of some vaccine applications, where delivery of antigen to the lysosome results in a greater T_{H}2 or antibody-mediated immune response. One can also take advantage of the ability of RTB to deliver across cell layers, a process called transcytosis, as well as into the target cells. For example (and as demonstrated in the examples below), the inventors discovered it is possible to employ the RTB to move the molecule of interest across cell layers. This is particularly useful, for example, where the target cell is an immune responsive cell that sits below a mucosal cell layer. Interaction with such specialized cells (such as M-cells) is necessary to interface with the key immune responsive cells. RTB can move the molecule of interest into the mucosal cell through endocytosis and cycle to the opposite cell surface, thereby interacting with the immune cells below. This is useful in applications where interaction with the specialized cells below the epithelial cells is useful, such as in mucosally-administered vaccines.

Further, separation of the RTB from the molecule of interest is possible after delivery to the target cell, by providing a disulfide bond between the RTB and molecule of interest. The target cell possesses various ways of breaking disulfide bonds. Various methods of accomplishing this are described below.

The foregoing provides an improved method of delivery of molecules of interest to a targeted cell. The method is useful in a wide range of applications. Examples, without intending to be limiting, include delivery of therapeutically useful molecules of interest to an animal cell, delivery of molecules of interest to the mucosal cells of an animal, drug delivery, vaccine antigen delivery, antibody delivery, nucleic acid delivery, and in treatment of diseases. RTB is very effective at carrying payload molecules across mucosal surfaces and thus could be enabling for nasal, oral, dermal, transdermal, inhalational, anal or vaginal delivery of certain drugs. Direct delivery of antigen molecules directly to antigen presenting cells (APCs) enhances the immune response. RTB has been shown to enhance immunogenicity of model antigens genetically fused to RTB (see Medina-Bolivar, et al. Vaccine 21 (2003) 997-1005). The conjugation chemistry potentially increases the payload to carrier ratio by up to eight-fold thus increasing efficacy of antigen presentation and vaccine efficacy, or effectiveness of drug delivery. Efficient delivery of DNA (gene therapy) or RNA (e.g., for RNAi strategies) into animal cells is achieved by linking the molecule of interest to RTB.

In one example of the many applications of use of the methods described is in treatment of lysosomal storage diseases. Lysosomes, which are present in all animal cells, are acidic cytoplasmic organelles that contain an assortment of hydrolytic enzymes. These enzymes function in the degradation of internalized and endogenous macromolecular substrates. When there is a lysosomal enzyme deficiency, the deficient enzyme's undegraded substrates gradually accumulate within the lysosomes causing a progressive increase in the size and number of these organelles within the cell. This accumulation within the cell eventually leads to malfunction of the organ and to the gross pathology of a lysosomal storage disease, with the particular disease depending on the particular enzyme deficiency. More than thirty distinct, inherited lysosomal storage diseases have been characterized in humans. One proven treatment for lysosomal storage diseases is enzyme replacement therapy in which an active form of the enzyme is administered directly to the patient. However, abundant, inexpensive and safe supplies of therapeutic lysosomal enzymes are not commercially available for the treatment of any of the lysosomal storage diseases. There are a large number of metabolic storage disorders known to affect man. As a group, these diseases are the most prevalent genetic abnormalities of humans, yet individually they are quite rare. One of the three major classes of these conditions, comprising the majority of patients, is the sphingolipidoses in which excessive quantities of undegraded fatty components of cell membranes accumulate because of inherited deficiencies of specific catabolic enzymes. Principal disorders in this category are Gaucher disease, Niemann-Pick disease, Fabry disease, and Tay-Sachs disease. All of these disorders are caused by harmful mutations in the genes that code for specific housekeeping enzymes within lysosomes. Thus, to be effective, enzyme replacement therapy requires that the requisite exogenous enzyme be taken up by the cells in which the materials are catabolized and that they be incorporated into lysosomes within these cells. Fabry disease is an ideal candidate for enzyme replacement therapy because the disease does not involve the central nervous system.

Examples of modified lysosomal enzyme are: (a) an enzymatically-active fragment of an N-acetylgalactosaminidase, acid lipase, α-galactosidase, glucocerebrosidase, α-L-iduronidase, iduronate sulfatase, α-mannosidase or sialidase; (b) the α-N-acetylgalactosaminidase, acid lipase, α-galactosidase, glucocerebrosidase, α-L-iduronidase, iduronate sulfatase, α-mannosidase, sialidase or (a) having one or more amino acid residues added to the amino or carboxyl terminus of the α-N-acetylgalactosaminidase, acid lipase α-galactosidase, glucocerebrosidase, α-L-iduronidase, iduronate sulfatase, α-mannosidase, sialidase or (a); or (c) the α-N-acetylgalactosaminidase, acid lipase, α-galactosidase, glucocerebrosidase, α-L-iduronidase, iduronate sulfatase, α-mannosidase, sialidase or (a) having one or more naturally-occurring amino acid additions, deletions or substitutions. The modified lysosomal enzyme can comprise: (a) an enzymatically-active fragment of a human glucocerebrosidase or human α-L-iduronidase enzyme; (b) the human glucocerebrosidase, human α-L-iduronidase or (a) having one or more amino acid residues added to the amino or carboxyl terminus of the human glucocerebrosidase, human α-L-iduronidase or (a); or (c) the human glucocerebrosidase, human α-L-iduronidase or (a) having one or more naturally-occurring amino acid additions, deletions or substitutions. The modified lysosomal enzyme can be a fusion protein comprising: (I) (a) the enzymatically-active fragment of the human or animal lysosomal enzyme, (b) the human or animal lysosomal enzyme, or (c) the human or animal lysosomal enzyme or (a) having one or more naturally-occurring amino acid additions, deletions or substitutions, and (II) a cleavable linker fused to the amino or carboxyl terminus of (I); and the method comprises: (a) recovering the fusion protein from the transgenic host cell, or the cell, tissue or organ of the transgenic host cell; (b) treating the fusion protein with a substance that cleaves the cleavable linker so that (1) is separated from the cleavable linker and any sequence attached thereto; and (c) recovering the separated (I).

RTB has utility for efficiently delivering enzymes or antigens into lysosomal compartments (e.g., replacement enzyme therapy for patients with lysosomal storage disorders). RTB binds surface glycoprotein and glycolipids that are present on most mammalian cell types, RTB has the potential to effectively deliver payload to all the key cells of pathological significance for lysosomal diseases by mechanisms that do not require expensive in vitro manipulation of lysosomal enzyme glycans (the mechanism utilized for replacement therapeutics such as Ceredase® (Genzyme) for Gaucher Disease).

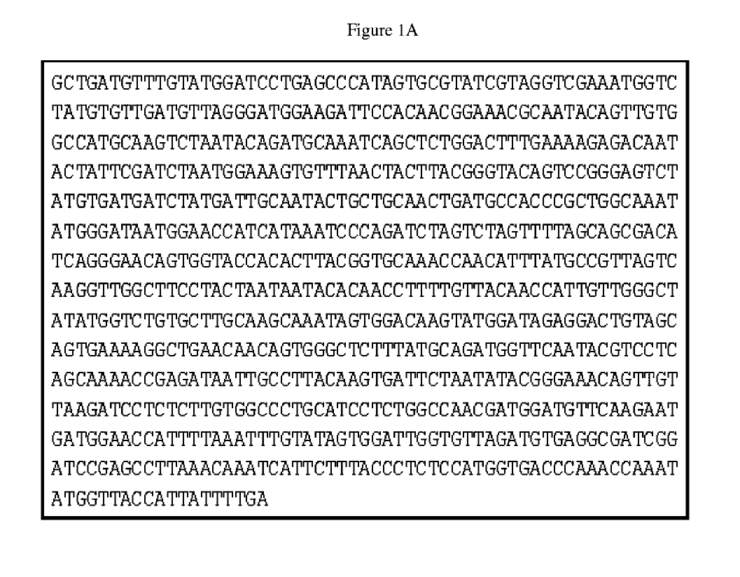

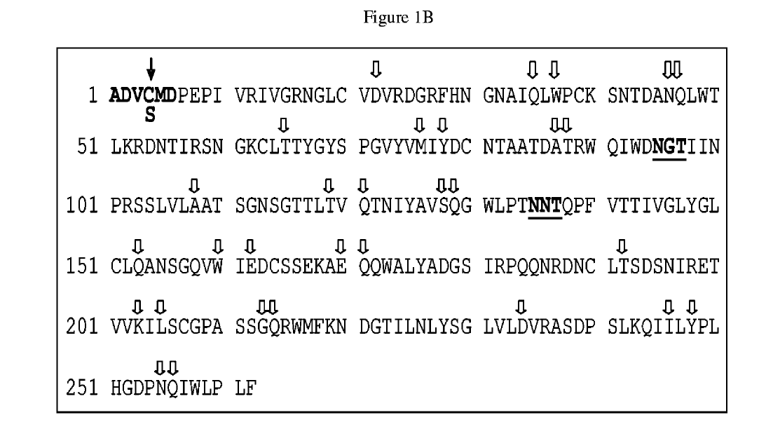

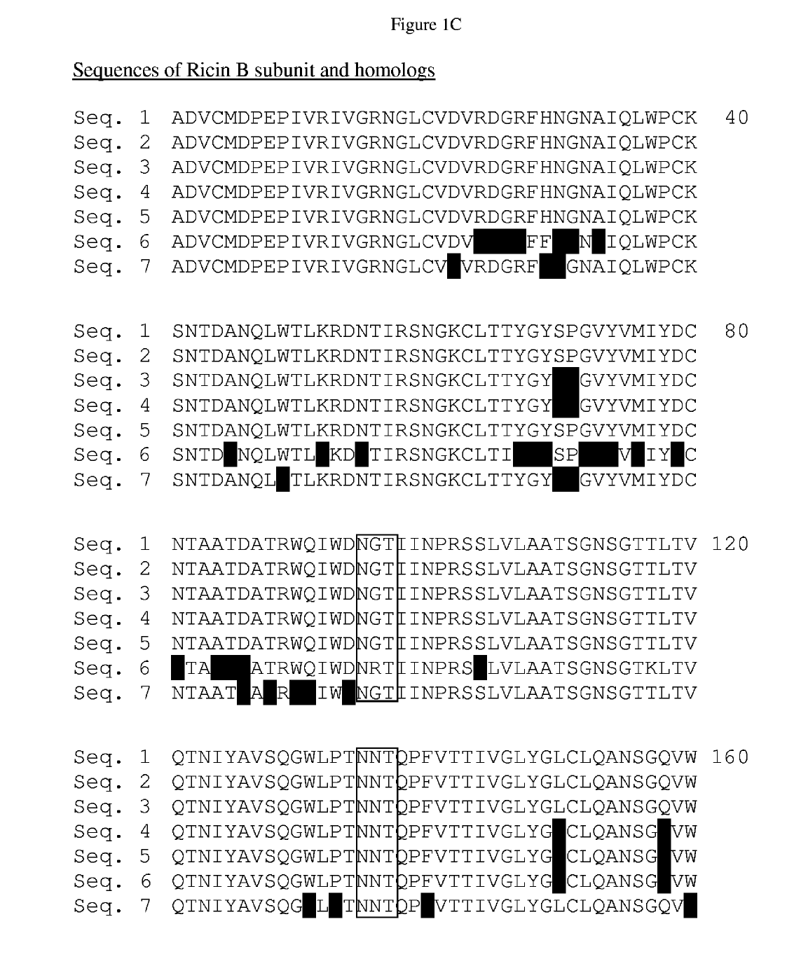

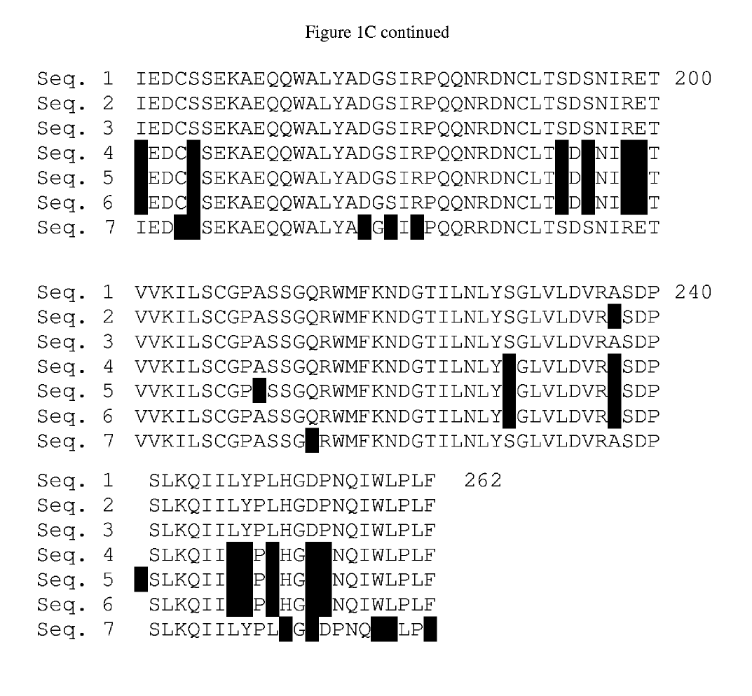

In referring to RTB is meant the ricin B chain that retains its lectin activity and does not contain the A chain. An exemplary nucleotide sequence encoding ricin B chain subunit is shown in FIG. 1A.

The ricin B chain protein is typically characterized as about 34,7000 daltons in molecular weight and about 260 to 262 amino acids and an exemplary sequence is shown in FIG. 1B. The first six bolded amino acids can be removed from the full chain (RTB) in select embodiments to make the truncated RTB. Those residues underlined are asparagine (N)-linked glycosylation sites. The black arrows point to the cysteine that forms a disulfide bond with the ricin A subunit (RTA) in the active toxin. In select embodiments, the cysteine (cys4) was modified to a serine to eliminate RTB-RTB dimerization or disulfide bonding to other molecules. In other embodiments, this cysteine can be used to associate molecules of interest to RTB via disulfide bonding. Open arrows indicate amino acids involved in sugar binding for lectin activity. (See Rutenberg et al. (1991) Structure of ricin B-chain at 2.5 A Resolution, Proteins: Structure, Function and Genetics, 10:260-269.) The actual amino acid sequence obtained from castor beans varies depending upon the variety, and recombinant ricin can be varied further for optimum performance. Those skilled in the art appreciate that the precise sequence will vary yet be able to retain its native biological activity. Here, when referring to the ricin B chain subunit nucleotide sequence is intended to include a nucleotide sequence encoding the ricin B chain, and when referring to the ricin B chain subunit is intended to include the amino acid sequence of same, and those substantially similar, that is, those able to carry out its native biological activity. By way of example, Piatak et al. describes in U.S. Pat. No. 5,840,522 differences between isoforms of ricin and sets forth an amino acid sequence for ricin E B chain subunit amino acid. Still other examples are shown in FIG. 1C which compares ricin B subunit amino acid sequences, including that of SEQ ID NO: 2 (sequence 1 in the figure) (See GenBank accession number pbd/2AAI/B, version GI:494727 (containing subunits A and B) by Montfort et al. and referenced at “The three-dimensional structure of ricin at 2.8 A” J. Biol Chem. 262 (11), 5398-5403 (1987); SEQ ID NO: 3 (sequence 2 in the figure) found at GenBank Accession No. gb/AAB22582.1 by Roberts et al. and referenced at Targeted Diagnostic Ther. 7, 81-97 (1992); SEQ ID NO: 4 (sequence 3 in the figure) GenBank Accession No. prf/1111227A, ricin D B chain by Araki et al. and referenced at FEBS Lett. 191 (1), 121-124 (1985); SEQ ID NO: 5 (sequence 4 in the figure) GenBank Accession No. prf/1303208A ricin E B chain by Araki et al. and discussed at Biochim. Biophys. Acta 911 (2), 191-200 (1987); SEQ ID NO: 6 (sequence 5 in the figure) GenBank Accession No. gb/AAA63506.1 a ricin E B chain by Ladin et al. and referenced at Plant Mol. Biol. 9, 287-295 (1987); SEQ ID NO: 7 (sequence 6 in the figure) GenBank Accession No. gb/AAB22584.1 from Ricinus communis by Roberts et al. and referenced at Targeted Diagn. Ther. 7, 81-97 (1992); and SEQ ID NO: 8 (sequence 7 in the figure) GenBank Accession No. prf/0702158A by ricin D by Kimura et al. and referenced at Agric. Biol. Chem. 45 (1), 277-284 (1981). The conserved N-linked glycosylation sites (NXT) are boxed. Amino acids that differ from SEQ ID NO: 2 are highlighted in gray. Thus one skilled in the art readily appreciates that the ricin B chain subunit referred to is the ricin B chain that retains its lectin activity and does not contain the A chain.

An RTB is produced by infecting or transforming the host cell with a nucleic acid molecule encoding the RTB and causing expression of the nucleic acid molecule such that RTB accumulates in the host cell. In referring to a recombinant RTB is meant an RTB other than the native ricin which is isolated from castor bean without further modification, and refers to an RTB produced without RTA. Nucleotide sequences encoding RTB are known, as discussed in the examples below. The methods are not limited to any particular sequence, as long as RTB is encoded which does not include the A-chain and retains lectin activity. The ELISA assay described below is one of a variety of methods available to one skilled in the art to ascertain lectin activity, such as affinity chromatography.

The RTB is operatively associated with the molecule of interest. In referring to operatively associated is intended any manner of associating the molecule with the RTB so that the molecule of interest is carried with the RTB to the target cells. This may occur, for example, after the RTB is recovered from the host cell, and can be associated with the molecule of interest using chemical interactions, for example. In another example, the RTB may be produced in the host cell fused to the molecule of interest, and then recovered.

Examples of such chemical interactions include conjugation, covalent binding, protein-protein interactions or the like. Examples of such in vitro operative associations are attachment of N-hydroxysuccinimide (NHS)-derivatized small molecules and proteins to recombinant RTB via formation of covalent interactions with primary amines. RTB contains eight primary amines, one at the N-terminus and seven on lysine side chains. Investigation of the crystal structure of RTB revealed that these lysines are situated on the surface of the molecule, thus allowing for a high payload to carrier ratio using this chemistry. Such modifications did not compromise RTB lectin activity or ability to deliver payload into mammalian cells. Another example utilizes NHS-biotin, which enables the RTB-mediated uptake of streptavidin (which binds strongly to biotin), allowing for attachment of up to eight copies of a large (˜60 kD in the case of streptavidin) protein to rRTB in this manner. Assembly of multiple large payloads onto RTB also did not compromise RTB lectin activity or its ability to deliver payload into mammalian cells. Hydrazine-derivatized small molecules, which form covalent binds to oxidized glycans on RTB, is another way to attach payloads to RTB.

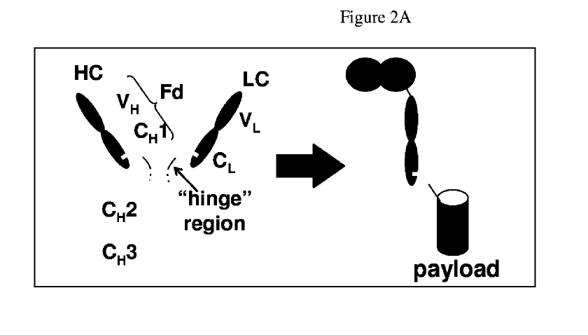

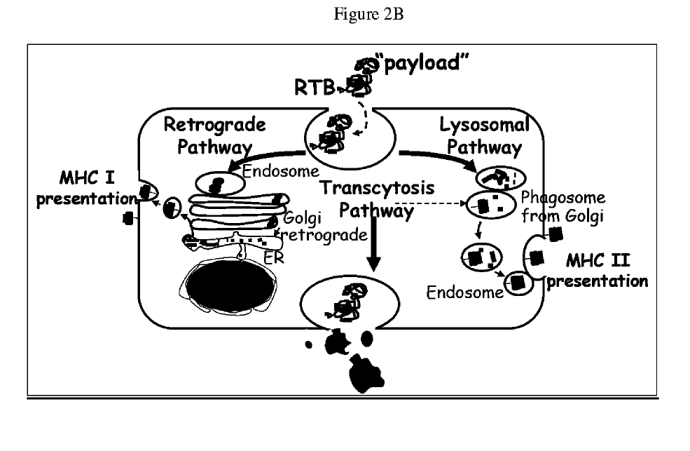

Immunoglobulins (Ig) are a class of proteins that are composed of two identical “heavy chains” and two identical “light chains”. One heavy chain is linked to one light chain through a disulfide bond, and the heavy chains are linked together via two disulfide bonds. An embodiment of the invention exploits these naturally-occurring interactions to attach a payload protein to RTB. The molecule of interest and the ricin subunit B are operatively associated with one another using this strategy by providing for a first immunoglobulin domain fused to the ricin B subunit. It is capable of assembling such that the molecule of interest and the subunit are operatively associated with one another. A diagrammatic representation of this interaction is shown in FIG. 2A. In one embodiment, the molecule of interest may itself comprise a second immunoglobulin domain which can assemble with the first immunoglobulin domain. In another embodiment, a second immunoglobulin domain is fused to the molecule of interest and the second immunoglobulin domain assembles with the first immunoglobulin domain. Clearly many variations on such a system are available to one skilled in the art.

In one example, this is accomplished by creating two DNA constructs that are co-expressed in a plant cell. Construct 1, the carrier construct, is a genetic fusion of the nucleotide sequence encoding RTB and the nucleotide sequence encoding the mouse kappa light chain, separated by a flexible (Gly₃Ser)₃ linker. When expressed alone, this construct produces the RTB:kLC protein. Construct 2, the payload construct, is a fusion of the Fd region of the mouse alpha heavy chain and the nucleotide sequence encoding the payload. When expressed alone, this construct produces Fd:payload. The Fd region is the portion of the heavy chain gene that interacts with the light chain. When constructs 1 and 2 are co-expressed in the same plant, the plant cell machinery assembles the two separate proteins into a heterodimer of RTB:kLC and Fd:payload joined via a disulfide bond. One skilled in the art could combine the relevant nucleic acid molecules which encode all the components on one construct, and the precise arrangement is dictated by the particular application. The RTB-specific purification protocol set out herein enables purification of this heterodimer regardless of the payload protein. Purified heterodimer is then endocytosed by target mammalian cells via RTB-mediated pathways. Referring to FIG. 2A, a representation of engineering an immunoglobulin domain-based scaffold to connect carrier to payload is represented. A typical Ig is depicted on the right. Individual domains are identified. The horizontal bars indicated inter-chain disulfide bonds. Each domain also contains either one or two internal disulfide bonds. The strategy exploits HC:LC interactions to mediate payload binding to an RTB-carrier protein via breakable disulfide interaction.

This technology is beneficial for a variety of reasons. First, because the carrier (RTB:kLC) and the payload (Fd:Payload) are joined through a breakable disulfide bond, release of the payload upon endocytosis is possible. This characteristic may be advantageous depending on the nature of the payload. Second, the system is flexible. One can change the carrier/payload identity merely by swapping out one payload gene for another. By including targeting information on the payload construct, localization is possible of the payload to a different compartment than that which the carrier would naturally migrate to Implementation of this technology increases the overall flexibility and efficiency of the RTB-mediated carrier system, and allows for payload- and application-specific carrier characteristics.

In the situation that it would be advantageous for a target payload protein to separate from the carrier after endocytosis, providing a breakable disulfide bond between the RTB and the molecule of interest allows for such separation. For example, the immunoglobulin-based scaffolding, as discussed above, is shown to be able to attach a payload protein to RTB through a breakable disulfide bond.

A nucleic acid encoding the RTB may also be expressed in the host cell as a fusion protein with the molecule of interest. Preparation of such constructs are well known to one skilled in the art and are described in further detail in examples below. The RTB in an embodiment may be expressed along with a sequence encoding an endoplasmic reticulum retrieval sequence. Any endoplasmic reticulum retrieval sequence may be used in the invention. One example is the KDEL sequence or the slightly longer version SEKDEL. The KDEL sequence (Lys-Asp-Glu-Leu) contains the binding site for a receptor in the endoplasmic reticulum. (Munro, S. and Pelham, H. R. B. 1987 “A C-terminal signal prevents secretion of luminal ER proteins.” Cell. 48:899-907.) Another example is HDEL, where histidine is substituted for the lysine.

In referring to a molecule of interest is intended any molecule which is capable of being operably associated with RTB and delivered by RTB to the target cells. (The term “payload” is used at times and refers to the molecule of interest). The molecule of interest selected will depend upon the ultimate intended use, but may include, for example, proteins; amino acids; enzymes (such as DNAase, and lysosomal and ER-localized enzymes used in replacement therapy); antigens (including both protein-based immunogens used, for example in cancer treatment and producing vaccines as well as chemical antigens used, for example in generating anti-chemical antibodies and for vaccine strategies targeting drugs of abuse); antibodies; cytokines; transcription factors; and polynucleic acids including deoxyribonucleic acid, ribonucleic acid, interfering ribonucleic acids; peptides; and chemicals and small molecules (such as therapeutic drugs, hormones, inhibitors, receptor agonists and antagonists, phytochemicals, antioxidants, and small molecular immunogens), among others.

A small sample of the type of polypeptides which may be useful in the invention include: various antibodies and antibody domains, cytokines [for example, interferon (IFN) α, β, γ, tumor necrosis factor (TNF) α, β, lymphotoxin (LT), interleukin (IL) 1-35, granulocyte colony-stimulating factor (G-CSF), granulocyte-macrophage colony-stimulating factor (GM-CSF), stem cell factor (SCF), leukemia inhibiting factor (LIF)], growth factors [for example, erythropoietin (EPO), nerve growth factor, epidermal growth factor (EGF), fibroblast growth factor (FGF), hepatocyte growth factor (HGF), platelet-derived growth factor (PDGF), vascular endothelial growth factor (VEGF), growth hormone (GH), insulin-like growth factor (IGF), transforming growth factors such as TGFα and TGFβ, and the like], vaccine antigens [for example, antigen proteins of human immunodeficiency virus (HIV), human hepatitis B virus (HBV), human hepatitis C virus (HCV), herpes simplex virus (HSV), cytomegalovirus (CMV), adult T cell leukemia virus (ATLV), influenza virus, Japanese encephalitis virus, rubella virus, measles virus, and the like including vaccine antigens targeting bacterial and parasitic diseases and vaccine antigens targeting diseases of livestock and poultry], various receptors [for example, G protein-coupled receptor (GPCR), cytokine receptor, nuclear receptor, and the like], various gene expression regulatory proteins, replacement enzyme therapeutics [for example lysosomal enzymes as discussed previously and enzymes with sites of action in the nucleus or endomembrane system such as UDG-glucuronosyltransferases], superoxide dismutase, α-1-antitrypsin, insulin, proinsulin, vesicle stimulating hormone, calcitonin, luteinizing hormone, glucagon, blood coagulation factors such as factor VIIIC, factor IX, factor Xa, tissue factor, and von Willebrand factor, anticoagulant factors such as protein C, atrial natriuretic factor, pulmonary surfactant, plasminogen activators such as urokinase and human urinary or tissue plasminogen activators (t-PA), bombesin, thrombin, enkephalinase, human macrophage inflammatory protein (MIP-1-α), mouse gonadotropin relating peptides, inhibin, activin, and the like. The foregoing is intended to be illustrative and not limiting of the type of molecules of interest useful in the invention.

As noted, the molecule of interest can be a nucleic acid molecule that does or does not produce a protein, and may inhibit expression of another molecule. Means of increasing or inhibiting a protein are well known to one skilled in the art and, by way of example, may include, transgenic expression, antisense suppression, co-suppression methods including but not limited to: RNA interference, gene activation or suppression using transcription factors and/or repressors, mutagenesis including transposon tagging, directed and site-specific mutagenesis, chromosome engineering (see Nobrega et. al., Nature 431:988-993(04)), homologous recombination, TILLING (Targeting Induced Local Lesions In Genomes), and biosynthetic competition to manipulate the expression of proteins. Many techniques for gene silencing are well known to one of skill in the art, including but not limited to knock-outs (such as by insertion of a transposable element such as Mu, Vicki Chandler, The Maize Handbook ch. 118 (Springer-Verlag 1994) or other genetic elements such as a FRT, Lox or other site specific integration site; RNA interference (Napoli et al. (1990) Plant Cell 2:279-289; U.S. Pat. No. 5,034,323, Sharp (1999) Genes Dev. 13:139-141, Zamore et al. (2000) Cell 101:25-33; and Montgomery et al. (1998) PNAS USA 95:15502-15507); virus-induced gene silencing (Burton, et al. (2000) Plant Cell 12:691-705, and Baulcombe (1999) Curr. Op. Plant Bio. 2:109-113); target-RNA-specific ribozymes (Haseloff et al. (1988) Nature 334: 585-591); hairpin structures (Smith et al. (2000) Nature 407:319-320; WO 99/53050; and WO 98/53083); MicroRNA (Aukerman & Sakai (2003) Plant Cell 15:2730-2741); ribozymes (Steinecke et al. (1992) EMBO J. 11:1525, and Perriman et al. (1993) Antisense Res. Dev. 3:253); oligonucleotide mediated targeted modification (e.g., WO 03/076574 and WO 99/25853); zinc-finger targeted molecules (e.g., WO 01/52620; WO 03/048345; and WO 00/42219); and other methods or combinations of the above methods known to those of skill in the art. Any method of increasing or inhibiting a protein can be used in the present invention. Several examples are outlined in more detail below for illustrative purposes.

The molecule of interest can be an antisense sequence for a targeted gene. (See, e.g., Sheehy et al. (1988) PNAS USA 85:8805-8809; and U.S. Pat. Nos. 5,107,065; 5,453,566; and 5,759,829). By “antisense DNA nucleotide sequence” is intended a sequence that is in inverse orientation to the 5′-to-3′ normal orientation of that nucleotide sequence. When delivered into a cell, expression of the antisense DNA sequence prevents normal expression of the DNA nucleotide sequence for the targeted gene. The antisense nucleotide sequence encodes an RNA transcript that is complementary to and capable of hybridizing with the endogenous messenger RNA (mRNA) produced by transcription of the DNA nucleotide sequence for the targeted gene. As noted, other potential approaches to impact expression of proteins in the plant include traditional co-suppression, that is, inhibition of expression of an endogenous gene through the expression of an identical structural gene or gene fragment introduced through transformation (Goring, D. R., Thomson, L., Rothstein, S. J. 1991. Proc. Natl. Acad Sci. USA 88:1770-1774 co-suppression; Taylor (1997) Plant Cell 9:1245; Jorgensen (1990) Trends Biotech. 8(12):340-344; Flavell (1994) PNAS USA 91:3490-3496; Finnegan et al. (1994) Bio/Technology 12: 883-888; and Neuhuber et al. (1994) Mol. Gen. Genet. 244:230-241)). In one example, co-suppression can be achieved by using a DNA segment such that transcripts of the segment are produced in the sense orientation and where the transcripts have at least 65% sequence identity to transcripts of the endogenous gene of interest, thereby suppressing expression of the endogenous gene in the cell. (See, U.S. Pat. No. 5,283,184). Additional methods of co-suppression are known in the art and can be similarly applied to the instant invention. These methods involve the silencing of a targeted gene by spliced hairpin RNA's and similar methods also called RNA interference and promoter silencing (see Smith et al. (2000) Nature 407:319-320, Waterhouse and Helliwell (2003)) Nat. Rev. Genet. 4:29-38; Waterhouse et al. (1998) Proc. Natl. Acad. Sci. USA 95:13959-13964; Chuang and Meyerowitz (2000) Proc. Natl. Acad. Sci. USA 97:4985-4990; Stoutjesdijk et al. (2002) Plant Phystiol. 129:1723-1731; and Patent Application WO 99/53050; WO 99/49029; WO 99/61631; WO 00/49035 and U.S. Pat. No. 6,506,559.

For mRNA interference, the expression cassette is designed to express an RNA molecule that is modeled on an endogenous siRNA gene. The siRNA gene encodes an RNA that forms a hairpin structure containing a 22-nucleotide sequence that is complementary to another endogenous gene. siRNA molecules are highly efficient at inhibiting the expression of endogenous genes.

In another example, the polynucleotide to be introduced into the cell comprises an inhibitory sequence that encodes a zinc finger protein that binds to a gene encoding a protein of the invention resulting in reduced expression of the gene. In particular embodiments, the zinc finger protein binds to a regulatory region of a gene of the invention. In other embodiments, the zinc finger protein binds to a messenger RNA encoding a protein and prevents its translation. Methods of selecting sites for targeting by zinc finger proteins have been described, for example, in U.S. Pat. No. 6,453,242.

Molecules of interest such as nucleic acid sequences (for example, single stranded RNAs, hairpin structure RNAs, double stranded RNAs, single stranded DNAs double stranded DNAs, and plasmid DNA) can be operatively linked the recombinant ricin B chain subunit utilizing conjugation chemistries referred to herein, including but not limited to bonding to primary amines, bonding to sugars of glycans on glycoproteins, and bonding through disulfide bond formation to cysteine residues of the protein (See, e.g., Lungwitz et al. (2005) Eur. J. Pharmacet. Bioparmacet. 60:247-266; Lovrinovic and Niemeyer (2005) BBRC 335:943-948). In one example, DNA sequences encoding a reporter protein such as β-glucuronidase (GUS) or green fluorescent protein (GFP) and operationally linked to promoter and terminator sequences for expression in plants are conjugated to RTB and RTB-ER as a double stranded DNA fragment and as part of an intact plasmid. The DNAs are initially conjugated to polyethylenimine (PEI) and the PEI-derivatized DNAs are then conjugated to the primary amines of the recombinant ricin B chain subunit. The RTB-DNA and RTB-ER-DNA products are then provided to a host cell(s), such as plant cells through various available processes that could include but are not limited to addition to plant protoplasts, infiltration into leaves or other tissue, rubbing on leaves with an abrasive, and particle bombardment. Delivery of the GFP-encoding DNA by RTB and rRTB-ER is detected as plant cells showing GFP fluorescence. Delivery of the GUS-encoding DNA by RTB and RTB-ER is detected as plant cells showing blue pigmentation following exposure to the GUS substrate X-Gluc. In another example, DNA linked to RTB is used to increase transformation efficiency in plants where vacuum infiltration of DNA is used and where particle bombardment is used to deliver DNA for transformation. In another example, DNA sequences are prepared for RTB-mediated delivery to animal cells, mucosal surfaces, tissues or organs in vitro or in vivo. DNAs containing operative elements for expression in animal cells or for integration into the animal cell genome are conjugated to RTB and RTB-ER as described above in the example targeting plant cells. In one example, DNA plasmids with sequences encoding GFP operationally linked to a strong constitutive CMV promoter (pGFP) are conjugated to the primary amines of RTB and RTB-ER using commercially available conjugation chemistries and linkers. In one embodiment, the RTB:pGFP and RTB-ER:pGFP products are added to cultured HT-29 and the cells are monitored over the next 48 hours for accumulation of GFP protein as monitored by fluorescence microscopy. In another example, mice are exposed intranasally and inhalationally to solutions containing RTB:pGFP and RTB-ER:pGFP. After 48, 72 and 96 hours, the presence of GFP proteins is monitored in situ using a whole animal imaging system. Alternatively, mice are sacrificed and nasal, bronchial and lung tissue is excised and analyzed by fluorescence microscopy. Target cells are the cells in which the molecule of interest is to be delivered.

Included in reference to target cell is a cell compartment or component, as discussed herein, such as the cytosol, lysosome, endoplasmic reticulum, vacuole or the like. It is intended to include targeting to combinations of cells such as a particular animal organ or tissue or types of cells such as macrophages or the like. Delivery can be across layers of cells, through transcytosis, and across the mucosa, as discussed further herein. In preferred embodiments, the cells are eukaryotic cells and in preferred embodiments are animal cells and in still further preferred embodiments are mammalian cells. The RTB and molecules of interest may be introduced to the target cells in any manner which provides optimum exposure of the RTB and molecule of interest to the cell. By introducing the RTB and the molecule of interest to the cell is meant a method of providing the RTB and molecule of interest such that the RTB is able to deliver the molecule of interest to the cell. Sufficient contact with the cells of the animal is needed in order that the RTB may interact with the cell. The methods here are especially useful where presenting the RTB and molecule to a mucosal cell of an animal and can include nasal, oral, dermal, transdermal, inhalational, anal or vaginal exposure. Clearly, the precise means of introducing the RTB and molecule to the target cell are not critical as long as the RTB can deliver the molecule to the cell. Injection, transformation and any other means of introducing the RTB and molecule to the cell are among the many methods available to one skilled in the art.

Host cells are those cells in which at least the recombinant rRTB may be expressed, and are those in preferred embodiments where the RTB and the molecule of interest may be expressed. They can be any eukaryotic cells, and include plant, insect, animal and yeast cells. In a preferred embodiment the host cell is a plant cell. When referring to a host cell it is also intended to include protoplasts, that is a cell consisting of the cell membrane and all of the intracellular components, but devoid of a cell wall.

The expression of RTB with or without expression of the molecule of interest in the host cell may be accomplished by preparing a construct comprising a nucleic acid molecule encoding the same. Clearly, one skilled in the art appreciates that such constructs can take many different forms and yet achieve the goal of expressing the RTB either with or without the molecule of interest and any other desired components. A promoter may be used to drive expression of one or more of the above. A selection marker may optionally be included. In other embodiments, the expression construct can contain two or more nucleotide sequences encoding RTB and/or the molecule of interest, which could be linked to the same promoter or different promoters. What is more, as discussed below, viral replication in plants may employ entirely different delivery methods. The precise methodology used will vary depending upon the goal to be achieved.

Promoter elements can be those that are constitutive or sufficient to render promoter-dependent gene expression controllable as being cell-type specific, tissue-specific or time or developmental stage specific, or being inducible by external signals or agents. Promoter elements employed to control expression of product proteins and the selection gene can be any host-compatible promoters. When used with plant host cells, these can be plant gene promoters, such as, for example, the ubiquitin promoter (European patent application no. 0 342 926); the promoter for the small subunit of ribulose-1,5-bis-phosphate carboxylase (ssRUBISCO) (Coruzzi et al., 1984; Broglie et al., 1984); or promoters from the tumor-inducing plasmids from Agrobacterium tumefaciens, such as the nopaline synthase, octopine synthase and mannopine synthase promoters (Velten and Schell, 1985) that have plant activity; or viral promoters such as the cauliflower mosaic virus (CaMV) 19S and 35S promoters (Guilley et al., 1982; Odell et al., 1985), the figwort mosaic virus FLt promoter (Maiti et al., 1997) or the coat protein promoter of TMV (Grdzelishvili et al., 2000). Still other promoter examples include an early or late promoter of adenovirus (Ad), an early or late promoter of simian virus 40 (SV40), a thymidine kinase (tk) gene promoter of herpes simplex virus (HSV), promoters obtained from viral genomes of Rous sarcoma virus, cytomegalovirus, mouse papilloma virus, bovine papilloma virus, avian sarcoma virus, retrovirus, hepatitis B virus and the like, promoters derived from mammals such as an actin promoter or an immunoglobulin promoter, and heat shock protein promoter.

The range of available host compatible promoters includes tissue specific and inducible promoters. An inducible regulatory element is one that is capable of directly or indirectly activating transcription of one or more DNA sequences or genes in response to an inducer. In the absence of an inducer the DNA sequences or genes will not be transcribed. Typically the protein factor that binds specifically to an inducible regulatory element to activate transcription is present in an inactive form, which is then directly or indirectly converted to the active form by the inducer. The inducer can be a chemical agent such as a protein, metabolite, growth regulator, herbicide or phenolic compound or a physiological stress imposed directly by heat, cold, salt, or toxic elements or indirectly through the actin of a pathogen or disease agent such as a virus. A host cell containing an inducible regulatory element may be exposed to an inducer by externally applying the inducer to the cell or plant such as by spraying, watering, heating or similar methods.

Any inducible promoter can be used in the instant invention. See Ward et al. (1993). Exemplary inducible promoters include ecdysone receptor promoters, U.S. Pat. No. 6,504,082; promoters from the ACE1 system which responds to copper (Mett et al. (1993)); In2-1 and In2-2 gene which respond to benzenesulfonamide herbicide safeners (U.S. Pat. No. 5,364,780; the GST promoter, which is activated by hydrophobic electrophilic compounds that are used as pre-emergent herbicides; and the PR-1a promoter, which is activated by salicylic acid. Other chemical-regulated promoters of interest include steroid-responsive promoters (see, for example, the glucocorticoid-inducible promoter in Schena et al. (1991) and McNellis et al. (1998) and tetracycline-inducible and tetracycline-repressible promoters (see, for example, Gatz et al. (1991), and U.S. Pat. Nos. 5,814,618 and 5,789,156).

Constitutive promoters can be utilized to target enhanced transcription and/or expression within a particular host tissue. Promoters may express in the tissue of interest, along with expression in other tissue, may express strongly in the tissue of interest and to a much lesser degree than other tissue, or may express highly preferably in the tissue of interest. Constitutive promoters include those described in Yamamoto et al. (1997); Kawamata et al. (1997); Hansen et al. (1997); Russell et al. (1997); Rinehart et al. (1996); Van Camp et al. (1996); Canevascini et al. (1996); Yamamoto et al. (1994); Lam (1994); Orozco et al. (1993); Matsuoka et al. (1993); and Guevara-Garcia et al. (1993).

The expression cassette may also include at the 3′ terminus of the isolated nucleotide sequence of interest, a transcriptional and translational termination region functional in the host. The termination region can be native with the promoter nucleotide sequence of the present invention, can be native with the DNA sequence of interest, or can be derived from another source. Thus, any convenient termination regions can be used in conjunction with the promoter of the invention, and are available from the Ti-plasmid of A. tumefaciens, such as the octopine synthase and nopaline synthase termination regions. See also: Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. 1989) Nucleic Acids Res. 17:7891-7903; Joshi et al. (1987) Nucleic Acid Res. 15:9627-9639.

The expression cassettes can additionally contain 5′ leader sequences. Such leader sequences can act to enhance translation. Translation leaders are known in the art and include: picornavirus leaders, for example, EMCV leader (Encephalomyocarditis 5′ noncoding region), Elroy-Stein et al. (1989) Proc. Natl. Acad. Sci. USA 86:6126-6130; potyvirus leaders, for example, TEV leader (Tobacco Etch Virus), Allison et al. (1986); MDMV leader (Maize Dwarf Mosaic Virus), Virology 154:9-20; human immunoglobulin heavy-chain binding protein (BiP), Macejak et al. (1991) Nature 353:90-94; untranslated leader from the coat protein mRNA of alfalfa mosaic virus (AMV RNA 4), Jobling et al. (1987) Nature 325:622-625); tobacco mosaic virus leader (TMV), Gallie et al. (1989) Molecular Biology of RNA, pages 237-256; and maize chlorotic mottle virus leader (MCMV), Lommel et al. (1991) Virology 81:382-385. See also Della-Cioppa et al. (1987) Plant Physiology 84:965-968. The cassette can also contain sequences that enhance translation and/or mRNA stability such as introns.

In those instances where it is desirable to have an expressed product of an isolated nucleotide sequence directed to a particular organelle, particularly the plastid, amyloplast, or to the endoplasmic reticulum, or secreted at the cell's surface or extracellularly, the expression cassette can further comprise a coding sequence for a transit peptide. Such transit peptides are well known in the art and include, but are not limited to: the transit peptide for the acyl carrier protein, the small subunit of RUBISCO, plant EPSP synthase, and the like. As noted herein, in particular applications of the invention an endoplasmic reticulum targeting sequence is provided preferentially directing expression to the endoplasmic reticulum of the cell. Signal peptides are also employed in embodiments of the invention, and are useful particularly when expressing RTB in the host cell and achieving proper folding of the RTB protein. Targeting may also be used in delivery of the RTB and the molecule of interest in the target cell. Any functional signal peptide will function for this purpose. For various reasons, targeting to other cellular components may also be desired. A variety of such sequences are known to those skilled in the art. For example, if it is preferred that expression be directed to the cell wall, this may be accomplished by use of a signal sequence and one such sequence is the barley alpha amylase signal sequence, (Rogers, (1985) J. Biol Chem 260, 3731-3738). Another example is the brazil nut protein signal sequence when used in canola or other dicotyledons. Directing expression with nuclear localization signals may also be useful. Examples of such applications are where transcription factor payloads are to be delivered to the nucleus, and when using zinc fingers, as discussed below. Such nuclear localization signals are know, such as Pro-Lys-Lys-Lys-Arg-Lys-Val which can act as a nuclear location signal. Kalderon et al. (1984) “A short amino acid sequence able to specify nuclear location” Cell 39 (3 Pt 2): 499-509. Expressing the protein in the endoplasmic reticulum of the host cell is accomplished through various sequences available. This may be accomplished by use of a localization sequence, such as KDEL. This sequence contains the binding site for a receptor in the endoplasmic reticulum. Munro, S. and Pelham, H. R. B. (1987) Cell 48:899-907. The patatin signal sequence is also frequently employed in cell expression. Iturriaga et al. (1989) The Plant Cell, Vol. 1, 381-390.

In preparing the expression cassette, the various nucleic acid fragments can be manipulated, so as to provide for the sequences in the proper orientation and, as appropriate, in the proper reading frame. Toward this end, adapters or linkers can be employed to join the fragments or other manipulations can be involved to provide for convenient restriction sites, removal of superfluous DNA, removal of restriction sites, or the like. For this purpose, in vitro mutagenesis, primer repair, restriction digests, annealing, and resubstitutions such as transitions and transversions, can be involved.

Reporter genes can be included in the transformation vectors. Examples of suitable reporter genes known in the art can be found in, for example: Jefferson et al. (1991) in Plant Molecular Biology Manual, ed. Gelvin et al. (Kluwer Academic Publishers), pp. 1-33; DeWet et al. (1987) Mol. Cell. Biol. 7:725-737; Goff et al. (1990) EMBO J. 9:2517-2522; Kain et al. (1995) BioTechniques 19:650-655; and Chiu et al. (1996) Current Biology 6:325-330. Selectable marker genes for selection of transformed cells or tissues can be included construct. These can include genes that confer antibiotic resistance or resistance to herbicides. Examples of suitable selectable marker genes include, but are not limited to: genes encoding resistance to kanamycin including neomycin phosphotransferase, see, e.g., Fraley et al, (1983) Proc. Natl. Acad. Sci. USA 80:4803; Miki et al. (1993) “Procedures for Introducing foreign DNA into plants” Methods in Plant Molecular Biology and Biotechnology”, Glick et al. (eds.) pp. 67-68 (CRC Press 1993); chloramphenicol, Herrera Estrella et al. (1983) EMBO J. 2:987-992; methotrexate, Herrera Estrella et al. (1983) Nature 303:209-213; Meijer et al. (1991) Plant Mol. Biol. 16:807-820; hygromycin, Waldron et al. (1985) Plant Mol. Biol. 5:103-108; Zhijian et al. (1995) Plant Science 108:219-227; streptomycin, Jones et al. (1987) Mol. Gen. Genet. 210:86-91; spectinomycin, Bretagne-Sagnard et al. (1996) Transgenic Res. 5:131-137; bleomycin, Hille et al. (1990) Plant Mol. Biol. 7:171-176; sulfonamide, Guerineau et al. (1990) Plant Mol. Biol. 15:127-136; bromoxynil, Stalker et al. (1988) Science 242:419-423; glyphosate, Shaw et al. (1986) Science 233:478-481; phosphinothricin, DeBlock et al. (1987) EMBO J. 6:2513-2518.

Expression of a linked sequence can be tracked by providing a useful so-called screenable or scorable markers. The expression of the linked protein can be detected without the necessity of destroying tissue. By way of example without limitation, detectable markers include a β-glucuronidase, or uidA gene (GUS), which encodes an enzyme for which various chromogenic substrates are known (Jefferson, R. A. et al., 1986, Proc. Natl. Acad. Sci. USA 83:8447-8451); chloramphenicol acetyl transferase; alkaline phosphatase; a R-locus gene, which encodes a product that regulates the production of anthocyanin pigments (red color) in tissues (Dellaporta et al., in Chromosome Structure and Function, Kluwer Academic Publishers, Appels and Gustafson eds., pp. 263-282 (1988); Ludwig et al. (1990) Science 247:449); a p-lactamase gene (Sutcliffe, Proc. Nat'l. Acad. Sci. U.S.A. 75:3737 (1978)), which encodes an enzyme for which various chromogenic substrates are known (e.g., PADAC, a chromogenic cephalosporin); a xylE gene (Zukowsky et al., Proc. Nat'l. Acad. Sci. U.S.A. 80:1101 (1983)), which encodes a catechol dioxygenase that can convert chromogenic catechols; an α-amylase gene (Ikuta et al., Biotech. 8:241 (1990)); a tyrosinase gene (Katz et al., J. Gen. Microbiol. 129:2703 (1983)), which encodes an enzyme capable of oxidizing tyrosine to DOPA and dopaquinone, which in turn condenses to form the easily detectable compound melanin a green fluorescent protein (GFP) gene (Sheen et al., Plant J. 8(5):777-84 (1995)); a lux gene, which encodes a luciferase, the presence of which may be detected using, for example, X-ray film, scintillation counting, fluorescent spectrophotometry, low-light video cameras, photon counting cameras or multiwell luminometry (Teeri et al. (1989) EMBO J. 8:343); DS-RED EXPRESS (Matz, M. V. et al (1999) Nature Biotech. 17:969-973, Bevis B. J et al. (2002) Nature Biotech 20:83-87, Haas, J. et al. (1996) Curr. Biol. 6:315-324); Zoanthus sp. yellow fluorescent protein (ZsYellow) that has been engineered for brighter fluorescence (Matz et al. (1999) Nature Biotech. 17:969-973, available from BD Biosciences Clontech, Palo Alto, Calif., USA, catalog no. K6100-1); and cyan florescent protein (CYP) (Bolte et al. (2004) J. Cell Science 117:943-54 and Kato et al. (2002) Plant Physiol 129:913-42).

Any plant cell is useful as the host plant cell of the invention, whether monocot or dicot. Examples include corn (Zea mays), canola (Brassica napus, Brassica rapa ssp.), alfalfa (Medicago sativa), rice (Oryza sativa), rye (Secale cereale), sorghum (Sorghum bicolor, Sorghum vulgare), sunflower (Helianthus annuus), wheat (Triticum aestivum), soybean (Glycine max), tobacco (Nicotiana tabacum; N. benthamiana), potato (Solanum tuberosum), peanuts (Arachis hypogaea), cotton (Gossypium hirsutum), sweet potato (Ipomoea batatus), cassava (Manihot esculenta), coffee (Cofea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), oats, barley, vegetables, ornamentals, and conifers.

Animal cells may also be used as the host cells. Examples of useful mammalian host cells include simian kidney derived lines transformed with SV40 (COS7 cells), human embryonic renal lines (293 cells), baby hamster kidney cells (BHK cells), Chinese hamster ovary cells (CHO cells, particularly CHO (DHFR.sup.-) cells (ATCC, CRL-9096)), mouse Sertoli cells (TM4 cells), simian renal cells (CV1 cells), african green monkey renal cells (VERO cells), human uterocervical carcinoma cells (HeLa cells), canine renal cells (MDCK cells), buffalo rat liver cells (BRL3A cells), human pulmonary cells (W138 cells), human liver cells (HepG2 cells), TRI cells, MRC5 cells, FS4 cells, and the like. Furthermore, it is also possible to use myeloma cells used as cells for cell fusion, hybridoma cells obtained by fusing these cells with a variety of lymphocytes or spleen cells. In addition, the useful host cells for practice of the present invention include multipotential embryonic stem cells (ES cells). The ES cells can be obtained from pre-implantation embryo cultured in vitro. These cells can be cultured, and also differentiated in vitro (Evans, N. J. et al., Nature, 292, 154 156, 1981). Such ES cells can be derived from any one of various species including primates such as human, useful cattle such as cows, pigs, sheep and goat, rats, rabbits, mice, and the like. Among those, the ES cells derived from cattle such as cows, pigs, sheep and goat, which can be used as a host for producing a foreign protein and derived from experimental animals such as rats and mice are appropriate hosts. Transformed ES cells selected with the marker gene have high possibilities that the desired protein is highly expressed, and thus the desired protein can be expected to be expressed in a high level in an animal obtained with the transformed ES cells.

The particular transformation protocol will vary depending upon the host. In plants, suitable methods of transforming plant cells include microinjection, Crossway et al. (1986) Biotechniques 4:320-334; electroporation, Riggs et al. (1986) Proc. Natl. Acad. Sci. USA 83:5602-5606; Agrobacterium-mediated transformation, see for example, Townsend et al. U.S. Pat. Nos. 5,563,055; direct gene transfer, Paszkowski et al. (1984) EMBO J. 3:2717-2722; viral replication systems, Turpen et al, 6,660,500 and 6,462,255; and ballistic particle acceleration, see for example, Sanford et al. U.S. Pat. No. 4,945,050, Tomes et al. (1995) in Plant Cell, Tissue, and Organ Culture: Fundamental Methods, ed. Gamborg and Phillips (Springer-Verlag, Berlin); and McCabe et al. (1988) Biotechnology 6:923-926. Also see Weissinger et al. (1988) Annual Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio/Technology 6:923-926 (soybean); Datta et al. (1990) Bio/Technology 8:736-740 (rice); Klein et al. (1988) Proc. Natl. Acad. Sci. USA 85:4305-4309 (maize); Klein et al. (1988) Biotechnology 6:559-563 (maize); Klein et al. (1988) Plant Physiol. 91:440-444 (maize); Fromm et al. (1990) Biotechnology 8:833-839; Hooydaas-Van Slogteren et al. (1984) Nature (London) 311:763-764; Bytebier et al. (1987) Proc. Natl. Acad. Sci. USA 84:5345-5349 (Liliaceae); De Wet et al. (1985) in The Experimental Manipulation of Ovule Tissues, ed. G. P. Chapman et al. (Longman, New York), pp. 197-209 (pollen); Kaeppler et al. (1990) Plant Cell Reports 9:415-418; and Kaeppler et al. (1992) Theor. Appl. Genet. 84:560-566 (whisker-mediated transformation); D. Halluin et al. (1992) Plant Cell 4:1495-1505 (electroporation); Li et al. (1993) Plant Cell Reports 12:250-255 and Christou et al. (1995) Annals of Botany 75:407-413 (rice); Osjoda et al. (1996) Nature Biotechnology 14:745-750 (maize via Agrobacterium tumefaciens).

The plant cells that have been transformed may or may not be grown into plants in accordance with conventional methods. See, for example, McCormick et al. (1986) Plant Cell Reports 5:81-84. These plants can then be grown and pollinated with the same transformed strain or different strains.

Animal cells may be transformed using many of the methods above, such as electroporation, (Zimmermann, U., Biochim. Biophys. Acta, 694:227, 1982) and microinjection, for example (Capecchi, M R, Cell, 22:479, 1980), as well as the calcium phosphate precipitation method (Graham, van der Eb, Virology, 52:456, 1973) and the liposome method (Mannino, R. J., Gould-Fogerite, S., BioTechniques, 6:682, 1988), and the like.

Expression vectors for the transformation of insect cells, and in particular, baculovirus-based expression vectors, are well known in the art. Transformation of insect cells and production of foreign polypeptides therein is disclosed by Guarino et al., U.S. Pat. No. 5,162,222 and WIPO publication No. WO 94/06463. Insect cells can be infected with recombinant baculovirus, commonly derived from Autographa californica nuclear polyhedrosis virus (AcNPV). See, King, L. A. and Possee, R. D., The Baculovirus Expression System: A Laboratory Guide, London, Chapman & Hall; O'Reilly, D. R. et al., Baculovirus Expression Vectors: A Laboratory Manual, New York, Oxford University Press., 1994; and, Richardson, C. D., Ed., Baculovirus Expression Protocols. Methods in Molecular Biology, Totowa, N J, Humana Press, 1995. The second method of making recombinant baculovirus utilizes a transposon-based system described by Luckow (Luckow, V. A, et al., J Virol 67:4566 79, 1993). This system is sold in the Bac-to-Bac kit (Life Technologies, Rockville, Md.). This system utilizes a transfer vector, pFastBac1™ (Life Technologies) containing a Tn7 transposon to move the DNA encoding the β-glucanase fusion protein into a baculovirus genome maintained in E. coli as a large plasmid called a “bacmid.” The pFastBac1™ transfer vector utilizes the AcNPV polyhedrin promoter to drive the expression of the gene of interest.

Fungal cells, including yeast cells, can also be used within the present invention. Yeast expression systems are well known in the art, and include expression vectors for Saccharomyces cerevisiae, Candida albicans and C. maltosa, Hansenula polymorpha, Kluyveromyces fragilis and K. lactis, Pichia guillerimondii and P. pastoris, Schizosaccharomyces pombe, and Yarrowia lipolytica. Some examples of methods employed are those for transforming disclosed by, Kawasaki, U.S. Pat. No. 4,599,311; Kawasaki et al., U.S. Pat. No. 4,931,373; Brake, U.S. Pat. No. 4,870,008; Welch et al., U.S. Pat. No. 5,037,743; and Murray et al., U.S. Pat. No. 4,845,075. Transformed cells are selected by phenotype determined by the selectable marker, commonly drug resistance or the ability to grow in the absence of a particular nutrient (e.g., leucine). A preferred vector system for use in Saccharomyces cerevisiae is the POT1 vector system disclosed by Kawasaki et al. (U.S. Pat. No. 4,931,373), which allows transformed cells to be selected by growth in glucose-containing media. Suitable promoters and terminators for use in yeast include those from glycolytic enzyme genes (see, e.g., Kawasaki, U.S. Pat. No. 4,599,311; Kingsman et al., U.S. Pat. No. 4,615,974; and Bitter, U.S. Pat. No. 4,977,092) and alcohol dehydrogenase genes. See also U.S. Pat. Nos. 4,990,446; 5,063,154; 5,139,936 and 4,661,454. Transformation systems for other yeasts, including Hansenula polymorpha, Schizosaccharomyces pombe, Kluyveromyces lactis, Kluyveromyces fragilis, Ustilago maydis, Pichia pastoris, Pichia guillermondii and Candida maltosa are known in the art. See, for example, Gleeson et al., J. Gen. Microbiol. 132:3459 65, 1986 and Cregg, U.S. Pat. No. 4,882,279. Aspergillus cells may be utilized according to the methods of McKnight et al., U.S. Pat. No. 4,935,349. Methods for transforming Acremonium chrysogenum are disclosed by Sumino et al., U.S. Pat. No. 5,162,228. Methods for transforming Neurospora are disclosed by Lambowitz, U.S. Pat. No. 4,486,533. Exemplary promoter sequences for expression in yeast include the inducible GAL1,10 promoter, the promoters from alcohol dehydrogenase, enolase, glucokinase, glucose-6-phosphate isomerase, glyceraldehyde-3-phosphate-dehydrogenase, hexokinase, phosphofructokinase, 3-phosphoglycerate mutase, pyruvate kinase, and the acid phosphatase gene. Yeast selectable markers include ADE2, HIS4, LEU2, TRP1, and ALG7, which confers resistance to tunicamycin; the neomycin phosphotransferase gene, which confers resistance to G418; and the CUP1 gene, which allows yeast to grow in the presence of copper ions.

Expression of the transferred sequence can be checked by detecting the marker gene, and various methods including, for example the northern blotting assay or RT-PCR with RNA recovered from cells, the ELISA assay or western blotting assay with an antibody of the expressed protein, or the detection of enzyme activity in the culture medium or cells in case of the protein being an enzyme.

The product of the sequence expression may or may not be isolated at this point. There are a variety of means available to one skilled in the art for purification of such products so expressed and any may be employed in the invention. For example, one could employ fractionation with affinity, immunoaffinity or ion exchange columns; ethanol, PEG, or ammonium sulfate precipitation; reverse phase HPLC; chromatography with cation exchange resins such as silica or DEAE, e.g. gel electrophoresis with Sephadex G-75; or chromatography with a plasminogen column to which the target product is linked. These methods can be referred to, for example, Guide to Protein Purification Methods in Enzymology, vol. 182, edited by Deutscher, Academic Press

The following examples are offered by way of illustration and not by way of limitation. All references cited herein are incorporated herein by reference.

### Example 1

Nature has evolved clever ways in which to deliver toxic proteins across the barrier of the cell membrane. Some of these toxins, such as the type II ribosome inactivating protein (RIP) ricin from the seeds of the castor bean plant (Ricinus communis), must navigate through labyrinthine paths once inside the cell to reach their substrate. In the case of ricin, it must reach the ribosomes which reside in the cytoplasm. Below is demonstrated that, by taking advantage of certain aspects of ricin function, such as the uptake and trafficking within target cells, while eliminating the toxic effects of ribosome inactivation, it is possible to create a safe and effective means to deliver proteins to cells which otherwise are not able pass the outer membrane barrier.

Ricin toxin is a heterodimeric protein consisting of the 32 kD N-glycosidase A-chain (RTA) which mediates ricin toxicity and the 32 kD galactose/N-acetyl-galactosamine-specific lectin B-chain (RTB), connected via a single disulfide bond.¹ Upon ingestion or inhalation of the toxin, ricin is taken up by cells through the binding of RTB to cell surface galactose residues. A large portion of endocytosed ricin remains in lysosomal and/or endosomal compartments.² However, some of the ricin RTA is delivered to the cytoplasm via retrograde transport through the Golgi to the endoplasmic reticulum (ER).³ Once in the ER, RTA and RTB dissociate⁴, and RTA is translocated to the cytoplasm via Sec61p-dependent pathways⁵, where it presumably evades ubiquitination and ER-associated degradation through a low abundance of lysine residues.⁶ Only then is RTA given access to the 28 S ribosomal RNA, upon which it de-purinates a specific nucleotide, halting protein synthesis.⁷

Researchers are exploring whether the trafficking of ricin within mammalian cells can be exploited for antigen or toxin delivery in immunological applications. One promising area of research is the use of RTA in immunotoxins, conjugated to monoclonal antibodies specific to certain cancer cell types.⁸ Other groups are interested in whether it might be possible to exploit the delivery potential of RTB to carry antigens or therapeutics across cell membranes. Early work by Roth, et al.^{9,10} and Hofmann, et al.^{11,12} demonstrated that insulin bound to RTB via disulfide bonds increased sensitivity in cell lines that were either sensitive or insensitive to insulin. However, the aim was not to deliver insulin as much as to facilitate binding to the cell surface to increase insulin:receptor interactions. More recently, Beaumelle, et al. showed that dihydrofolate reductase was transported to the cytosol (through an unfolded intermediate) of target cells when fused to the N-terminus of a disarmed RTA (RTA_{R180H}¹³) and re-associated with castor bean-derived RTB (cbRTB).¹⁴ Tagge et al. performed similar experiments with green fluorescent protein (GFP) fused to RTA_{R180H}, then reassembled with cbRTB, and was able to follow the trafficking in Hep3B and KB cells through fluorescence microscopy.¹⁵ Because antigen presentation through the Major Histocompatibility Complex I (MHC I) pathway also involves Sec61p transport to the cytosol, researchers have explored whether the ricin transport pathway could be exploited for vaccine strategies (see FIG. 2B). Grimaldi, et al. has shown that a short peptide of the mouse pneumovirus (P₂₆₁) fused to RTA_{R180H}, re-associated with RTB and delivered to mice intraperitoneally produced P₂₆₁-specific CD8⁺ T-cells, but did not protect against viral challenge. Results reported by this group were consistent with MHC class I presentation.¹⁶

The pathways in a ricin-mediated antigen delivery system which may be exploited are represented in FIG. 2B. Upon endocytosis, a large portion of ricin moves into the lysosomal pathway, and cycles between lysosomal and endosomal compartments, as well as the cell surface. In Antigen Presenting Cells (APC) this pathway leads to Major Histocompatibility Complex II presentation. Some internalized ricin moves into the Retrograde pathway, traveling through the Golgi to the ER (via RTB's interaction with calreticulin), where RTA and RTB dissociate and RTA is transported to the cytosol by Sec61p. In ACPs the retrograde pathway leads to MHC class I antigen presentation. In epithelial cells, the transcytosis pathway may lead to contact with and uptake by APCs.

RTB's specificity makes it uniquely suited to mucosal vaccines. Mucosal surfaces comprise the site of most infection and therefore the location where strong immunity is required. Medina-Bolivar, et al. demonstrated the adjuvancy of RTB when fused to the model antigen GFP. RTB:GFP fusions were produced in transgenic tobacco hairy root cultures and administered to mice intranasally. RTB mediated the induction of strong GFP-specific immune responses comparable to that of cholera toxin B, a model mucosal adjuvant.¹⁷ However, in the course of these experiments, cellular uptake of RTB:GFP was not characterized. Work by Choi, et al. also used only RTB and focused primarily on vaccine applications, concentrating on RTB's mucosal specificity. Researchers from this group have shown that an outer capsid glycoprotein (VP7) of simian rotavirus SA11 fused to the N-terminus of RTB is produced in potato with biological activity, but at low expression levels.¹⁸ They have also shown that the fusion of NSP4 peptide of rotavirus to RTB, expressed in E. coli as inclusion bodies and refolded, enhanced the immunogenicity of NSP4 in mice over NSP4 alone.¹⁹ Unpublished data from our group has shown that RTB does not exhibit adjuvancy effects when administered as an ad-mix (i.e. not genetically fused or otherwise conjugated to) with ovalbumin (OVA) in mice. In contrast, a stimulation of OVA-specific immune response was observed by OVA ad-mixed with mistletoe lectin I (MLI), a less toxic type II RIP. This data showed that toxicity plays a role in immune response. Given that RTB alone is non-toxic, we hypothesize that RTB's adjuvancy as observed by Medina-Bolivar and Choi is due to a direct delivery and presentation of antigens to antigen presenting cells (APCs).

In order to overcome problems of these prior approaches, a recombinant RTB (rRTB) was used as a platform for a flexible and efficient protein delivery system. Producing a recombinant form of RTB eliminates the requirement of separating RTB from RTA in ricin toxin, as the gene encoding RTA would never be present. This precludes the possibility of toxicity. Secondly, certain methods of attaching “payload” molecules and/or proteins to recombinant RTB (rRTB) were used and found that they did not interfere with RTB's lectin and cellular uptake functions. By way of example is described here: 1) the production of rRTB and RTB:GFP in Nicotiana benthamiana by Agrobacterium-mediated transient expression, and subsequent purification; 2) the fluorescein labeling and biotinylation of rRTB; and 3) the uptake of fluorescein-rRTB, rRTB-biotin complexed with fluorescein-streptavidin, and RTB:GFP into human epithelial HT-29 cells. This research shows that potential payloads can be connected to the RTB carrier through various ways, including chemical conjugation at primary amines and direct fusion of polypeptides. Also, payload molecules conjugated to streptavidin were carried into cells via binding to biotinylated RTB.

### Methods

### Gene Constructs

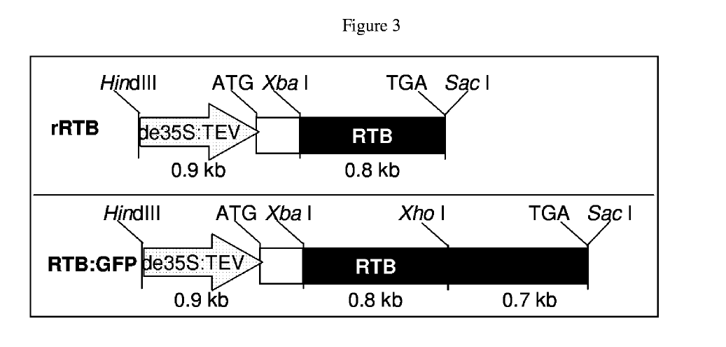

Maps of the constructs used in these studies are shown in FIG. 3. Recombinant RTB (rRTB) and RTB:GFP gene construct maps are shown. Both constructs are driven by the constitutive dual enhanced 35S CaMV promoter and contain the patatin signal peptide (sp). Constructs were assembled in pBC and promoter:gene cassettes were subcloned into the pBIB-Kan binary vector (via HindIII/SalI for rRTB and HindIII/SacI for RTB:GFP). The creation of construct R6-2, encoding RTB:GFP has been described elsewhere.¹⁷

Sequences encoding recombinant RTB (rRTB) were PCR amplified using 5′-TCTAGAGCTGATGTTTCTATGGAT (F) (SEQ ID NO: 9) and 5′-GTCGACTCAAAATAATGGTAACCATA (R) (SEQ ID NO: 10) using Pfu DNA polymerase. The template used was R6-2. These primers added the XbaI restriction site on the 5′ end of the gene (underlined), and a stop codon (TGA; in red) and SalI site on the 3′ end (bold). The HindIII/XbaI fragment containing the dual enhanced cauliflower mosaic virus (CaMV) 35S promoter, the tobacco etch virus (TEV) translational enhancer, and the patatin signal peptide (de35S:TEV::sp) was isolated from plasmid pBC-R6-2. The de35S:TEV::sp fragment and the rRTB PCR product were ligated into the pBC cloning vector (Stratagene, Cedar Creek Tex.) which was digested with HindIII and SalI in a tri-molecular reaction to give plasmid pBC-35S:rRTB. After sequence confirmation, the promoter:gene cassette was subcloned into the pBIB-Kan²⁰ binary vector via HindIII SalI.

### Plant Growth Conditions

Seeds of Nicotiana benthamiana, provided by Dr. S. Tolin (Virginia Tech, Blacksburg Va.), were germinated by direct-seeding into 4 inch pots and plants were used for expressing rRTB and RTB:GFP. The growth media used was a 2:1 mixture of Promix BX and PGX (Hummert). Growth conditions of 16 hr photoperiod (180 μmol s⁻² m⁻¹), 25° C. days, 21° C. nights, 65% humidity were maintained via Conviron ATC60 growth chamber. Plants were watered as needed. Plants 5-6 weeks from seeding were selected for infiltration. The typical yield of infiltrated leaf material was 10-20 g fresh weight per plant.

Agrobacterium tumefaciens-Mediated Transient Expression

pBIB-Kan plasmids harboring promoter:gene cassettes were transformed into A. tumefaciens strain LBA4404 using a modified freeze/thaw method.²¹ Positive clones were grown in 50 mL YEP medium (10 g/L bacto-peptone, 10 g/L yeast extract, 5 g/L NaCl) containing 100 μg/mL kanamycin and 60 μg/mL streptomycin for 48 hr at 28° C., 220 rpm. To induce A. tumefaciens prior to infiltration, cell pellets were harvested via centrifugation (5000×g for 10 min), resuspended in 300 mL induction media (20 mM MES pH 5.5, 0.3 g/L MgSO₄.7H₂O, 0.15 g/L KCl, 0.01 g/L CaCl₂, 0.0025 g/L FeSO₄.7H₂O, 2 mL/L 1 M NaH₂PO₄ pH 7.0, 10 g/L glucose) containing 100 μg/mL kanamycin and 60 μg/mL streptomycin, supplemented with 0.2 μM acetosyringone and incubated at 28° C., 220 rpm, for 4 hr to overnight. Induced A. tumefaciens cultures were introduced into four to six week old Nicotiana benthamiana plants either by pressure injection or vacuum infiltration. For pressure injection, a disposable syringe without a needle was filled with A. tumefaciens culture and pressed against the underside of the leaf.²² For vacuum infiltration, plants were place upside-down in a beaker containing the induced culture so that all aerial portions were submerged. This was then placed inside a vacuum chamber and vacuum was applied (approximately 1 min) and broken by abruptly pulling off the tube from the chamber.²³ This procedure was performed twice for each plant to ensure complete infiltration. Following infiltration, plants were replaced to their growth chambers and allowed to incubate for 48-72 hr.

### Extraction and Purification of RTB and RTB:GFP Fusion Proteins

For Agro-infiltrated leaves, 10-20 g of infiltrated leaves were ground under LN₂ in a mortar and pestle to a fine powder. 50 mL of extraction buffer 2 (100 mM Tris-HCl pH 7.5, 20 mM D-galactose, 1% PVPP) was added to the powder and allowed to thaw at RT. The resulting crude extract was centrifuged at 14,200×g for 30 min at 4° C. The supernatant was filtered through KimWipes, brought to 100 mL with distilled H₂O and the pH was adjusted to 7.5 with 1 N NaOH. This cleared extract was then filtered though a 0.45 μm membrane and loaded onto an equilibrated 20 mL column volume MacroPrep High Q (Bio-Rad, Hercules Calif.) column using a Bio-Rad Duo-Flow FPLC system. Following loading of the sample, the column was washed with 80 mL of 50 mM Tris-HCl pH 7.5. The RTB-containing proteins were eluted and collected from the column by washing with 45 mL 50 mM Tris-HCl pH 7.5, 400 mM NaCl. The column was then cleaned by washing with 50 mM Tris-HCl pH 7.5, 1 M NaCl and re-equilibrated with 50 mM Tris-HCl pH 7.5. The RTB-containing sample (400 mM NaCl) was loaded onto a 1 mL immobilized lactose column (EY Laboratories, San Mateo Calif.) and washed with PBS. Purified RTB and RTB-containing fusion proteins were eluted by washing with 4×1 mL PBS+500 mM D-galactose. RTB-containing samples were then concentrated using YM-10 Centricons (Millipore Corp., Bedford Mass.) and dialyzed to PBS. Concentrated, dialyzed samples were then analyzed via silver stained SDS-PAGE and asialofetuin binding assay. SDS-PAGE was performed using 10% or 12% PAGE-gels (PAGE-gel, Inc. San Diego, Calif.). Silver staining was performed using the SilverSnap kit (Pierce, Rockford, Ill.).

### Asialofetuin Binding Assay.

A functional ELISA utilizing asialofetuin instead of a capture antibody was employed to assess galactose-specific lectin activity and quantify rRTB and RTB-containing fusion proteins. Asialofetuin is a modified mammalian glycoprotein that contains galactose-terminated glycans (Sigma, St. Louis Mo.). Asialofetuin at 300 μg/mL in PBS was bound to the wells of an Immulon 4HBX plate for 1 hr at RT. The wells were then blocked with 3% BSA in PBS for 1 hr at RT. Castor bean-derived RTB (cbRTB; Vector Labs, Burlingame Calif.) was used for the standard curve, ranging from 1.95 to 250 ng/well in PBS+10 mM D-galactose. For asialofetuin binding, 100 μL of standards and samples incubated at RT for 1 hr. The plate was then washed 3× with PBS (300 μL/well). Rabbit anti-Ricinus communis lectin antibody (Sigma R-1254), diluted to 1:4000 in blocking buffer was then added (200 μL/well) and allowed to incubate for 1 hr at RT. Wells were washed again and alkaline phosphatase labeled goat anti-rabbit antibody (Bio-Rad, Hercules Calif.), diluted 1:4000 in blocking buffer, was added and allowed to incubate for 45 min at RT. The wells were washed a third time and alkaline phosphatase substrate (100 μL/well; Pierce, Rockford Ill.) was applied. After the color developed sufficiently (10-15 min), the reaction was stopped with the addition of 50 μL 2 N NaOH and the absorbance at 405 nm was read. The inverse of the absorbance at 405 nm was plotted vs. the inverse of the standard RTB/well to give a linear relationship. This equation was then used to estimate the amount of RTB in the samples in terms of RTB equivalents.

By probing with antibodies for proteins other than the rabbit anti-Ricinus communis lectin antibody used for quantification, it was possible to use this assay to determine carrier/payload interactions. For example, cbRTB and rRTB samples that underwent biotinylation chemistry (see below) were assessed for lectin activity and confirmation of biotinylation by probing with horse-radish peroxidase-labeled streptavidin (Sigma, St. Louis Mo.) instead of anti-ricin antibodies. By inclusion of the proper controls, a positive reaction in this scenario indicated that biotin must be present since binding to asialofetuin presumed the presence of RTB. Identical samples probed with anti-ricin antibodies confirmed this.

### Production of RTB Specific Antibodies

The 789 bp fragment encoding the RTB portion of ricin toxin was amplified from preproricin template using primers 5′-CATATGGCTGATGTTTGTATGGATC (F) (SEQ ID NO: 9) and 5′-5′-GTCGACTCAAAATAATGGTAACCATA (R) (SEQ ID NO: 10) to add NdeI (underlined) to the 5′ end and SalI to the 3′ end (bold). A stop codon (red) was added just upstream of the SalI site. This fragment was cloned into pET41 (EMD Biosciences, San Diego Calif.). The use of the NdeI site at the 5′ end removed the vector-encoded GST sequences, creating a RTB gene containing no vector-encoded tags. pET-RTB was transformed into E. coli strain BL21(DE3). A 5 mL overnight culture was used to inoculate 1 L LB containing 100 μg/mL kanamycin (2 L flask). The culture was grown at 37° C. (220 rpm) for ˜3 hr or until the OD₆₀₀ reached ˜0.8. Isopropyl β-D-1-thiogalactopyranoside (IPTG) was added to a final concentration of 1 mM and allowed to incubate at 37° C. (220 rpm) for 3 hr. The cells were harvested via centrifugation and lysed in 30 mL 1× BugBuster (EMD Biosciences) reagent supplemented with 1 mg/mL lysozyme, 10 μg/mL DNase 1,10 μg/mL RNase A. The crude extract was centrifuged at 13,000×g for 15 min Inclusion bodies were recovered; and there was no detectable RTB in the soluble fraction via Coomassie stained SDS-PAGE analysis of pre- and post-induction fractions. This is consistent of other reports of E. coli produced RTB.^{19,24} Inclusion bodies were washed 3× with 25 mL 50 mM Tris-HCl pH 8.0, 0.1% Triton X-100, then dissolved in 2 mL 50 mM CAPS pH 11.0, 0.3% N-laurylsarcosine, 5 mM DTT. This was dialyzed to PBS+5 mM DTT 2× at 4° C. The solubilized RTB migrated to the predicted molecular weight on SDS-PAGE but showed no activity via asialofetuin assay or immobilized lactose chromatography. This is most likely due to misfolding.

As an aside, it should be noted here that many attempts were made to optimize E. coli-derived rRTB, in terms of activity of refolded protein. No reports are known that have shown production of soluble rRTB in E. coli. Protocols reported by Tonevitsky, et al.²⁴, and Choi, et al.²⁵, as well several other methods not reported were tried, with limited success. Different chaotropic reagents such as urea, guanidine HCl, and N-laurylsarcosine were tested. Methods such as dilution and successive rounds of dialysis to remove the chaotropic reagent after solubilization of the inclusion bodies, and therefore induce refolding, were also tried. The addition of redox pairs such as reduced/oxidized glutathione and cysteine/cystine, as well as reductants such as DTT and βME also proved inefficient. The presence of galactose in the refolding buffer also did not produce significant quantities of active rRTB, in our hands. We therefore only used E. coli-derived rRTB for the production of antibodies.

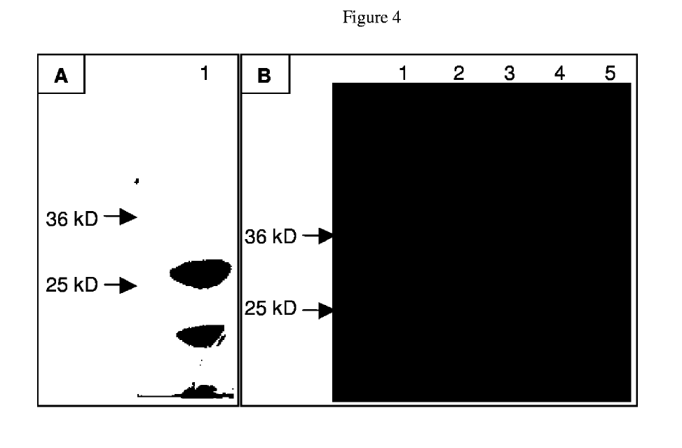

FIG. 4 shows generation of RTB-specific antibodies in rabbit. FIG. 4A shows Coomassie-stained SDS-PAGE (12%, reduced) gel of soluble/misfolded E. coli-derived RTB (lane 1) used as antigen in developing antibodies. FIG. 4B is a Western blot of plant-derived RTB probed with rabbit anti-RTB (1:10,000). Bands were developed using alkaline-phosphatase labeled goat anti-rabbit (1:4000) and CDP-Star. Lane 1 is cbRTB 30 ng; lane 2 is empty-vector (pBK) Agro-infiltrated leaf extract (10 μg total protein); lane 3 is pBK lactose elution fraction; lane 4 is 6HIS-RTB crude leaf extract (10 μg total protein); and lane 5 is 6HIS-RTB lactose elution fraction. Approximately 1 mg of solubilized inactive E. coli-derived RTB (FIG. 4A) was sent to CoCalico Biologicals (Reamstown Pa.) for raising an RTB-specific antibody in rabbit. Rabbit serum was tested for reactivity towards plant-derived RTB (cbRTB and 6HIS-RTB, a gift of Dr. Medina-Bolivar, described elsewhere²⁶) via western blots (see FIG. 4B). Total IgG was collected from the pooled positive sera via Protein A chromatography. After removing unbound material with PBS, bound IgG was eluted from Protein A sepharose beads (Sigma) by washing with 100 mM glycine pH 3.0 which was immediately neutralized with 1 M Tris-HCl pH 9.5. IgG fractions were pooled and concentrated via YM-30 Centricons (Millipore Corp., Bedford Mass.). Glycerol was added to a final concentration of 50% and antibodies were stored at −20° C. The titer for standard western blots is as high as 1:20,000 with sensitivity as low as 1 ng/lane.

### Fluorescein Labeling

cbRTB and purified rRTB were labeled at primary amine groups with NHS-Fluorescein (Pierce 46100) as per the manufacturer's instructions. The labeling reaction was allowed to proceed for 2 hr at RT. Unreacted NHS-fluorescein was quenched by the addition of Tris-HCl pH 7.5 to a final concentration of 50 mM. The reaction mixture was then dialyzed to PBS 2× at 4° C.

### Biotinylation of RTB and Binding to Streptavidin

cbRTB and rRTB were labeled at primary amine groups with sulfo-NHS-LC-biotin (Pierce 21335) as per the manufacturer's instruction. The labeling reaction was allowed to proceed for 2 hr at RT. Unreacted sulfo-NHS-LC-biotin was quenched by the addition of Tris-HCl pH 7.5 to a final concentration of 50 mM. The reaction mixture was then dialyzed to PBS 2× at 4° C. Biotinylation of RTB was confirmed via a modified asialofetuin assay. Briefly, RTB-biotin was applied to microtiter plate wells that were coated with asialofetuin and then blocked. This was allowed to incubate at RT for 1 hr, then washed with PBS. Horseradish peroxidase-labeled streptavidin (strep-HRP) was then applied to wells and allowed to incubate at RT for 20 min Horse radish peroxidase (HRP) substrate (KPL, Gaithersburg Md.) was added after washing and the color was allowed to develop for ˜10 min before stopping with 1 N H₂SO₄. The absorbance at 450 nm was then read. Only wells containing biotinylated RTB gave a reaction. The extent of biotinylation on a molar ratio basis was not determined. Prior to uptake, cbRTB-biotin and rRTB-biotin were mixed with fluorescein-labeled streptavidin (Sigma S-3762).

### Cell Uptake and Fluorescence Microscopy

For experiments to test the cellular uptake function of the various carrier/payload schemes developed, we used HT-29 human epithelial cells (ATCC) grown to 50-75% confluence in individual wells of an optical-bottom 96-well-microtiter plate (#165305; Nalge Nunc International, Rochester N.Y.) in McCoy's 5A media (+fetal bovine serum; Invitrogen, Carlsbad Calif.). Prior to sample loading, cells were washed 3× with ice-cold Hank's Balanced Salt Solution (HBSS; Invitrogen). Samples for uptake were brought to 100 μL with either HBSS or PBS before adding to the cells. Samples and cells were incubated at 4° C. for 30 min to allow binding to the cell surface without being endocytosed. Cells were then washed 3× with ice-cold HBSS again to remove any unbound proteins. Ice-cold HBSS (200 μL/well) was then added, and the T=0 picture taken. Fluorescence microscopy was performed using a Zeiss Axiovert 200M microscope fitted with SensiCam QE digital camera, and utilized IP Lab software. Photographs were taken at 40× magnification using a GFP UV filter set. The plate containing the cells was then incubated at 37° C., 5% CO₂ (normal growth conditions) to initiate endocytosis. Additional pictures were taken at T=30, 60 and 120 min

### Results

Expression and Purification of rRTB and RTB:GFP

In order to have a flexible source of rRTB and RTB-fusions, an Agrobacterium-mediated transient expression system using Nicotiana benthamiana was optimized. Efforts to develop a bacteria-based rRTB production system did not yield sufficient lectin-active rRTB. rRTB produced in E. coli was almost completely found in insoluble inclusion bodies. Re-naturation of this insoluble rRTB to an active form was inefficient (see Methods), and the feasibility of such a system on a commercial scale is low. The ideal production system would be a plant-based one, since RTB is a plant protein. In addition, bacterial systems such as E. coli do not have the capability to perform post-translational glycosylation. It has been reported by Frankel, et al. that the mannose-containing glycans on RTB facilitate uptake through the cell-surface mannose receptor in the absence of RTB lectin activity.²⁷ This suggests that presence of the glycan is important in providing yet another route for RTB to enter the cell and deliver payload proteins. To the author's knowledge, no type II RIP has been discovered in Nicotiana spp., suggesting that rRTB produced in N. benthamiana is unlikely to interact with an endogenous type II RIP A-chain.

By infiltrating Agrobacterium tumefaciens cultures directly into the leaves of N. benthamiana, a short (up to 5-7 days) burst of transient expression of genes residing in the T-DNA portion of plasmids carried by the A. tumefaciens occurs. This short expression window allows testing of a large number of gene constructs in a short time, relative to stable transformation. Due to this flexibility and the observation that expression levels of transiently expressed genes were higher than the same gene stably transformed, we chose to utilize this Agrobacterium-mediated transient expression of rRTB and RTB-fusions in N. benthamiana.

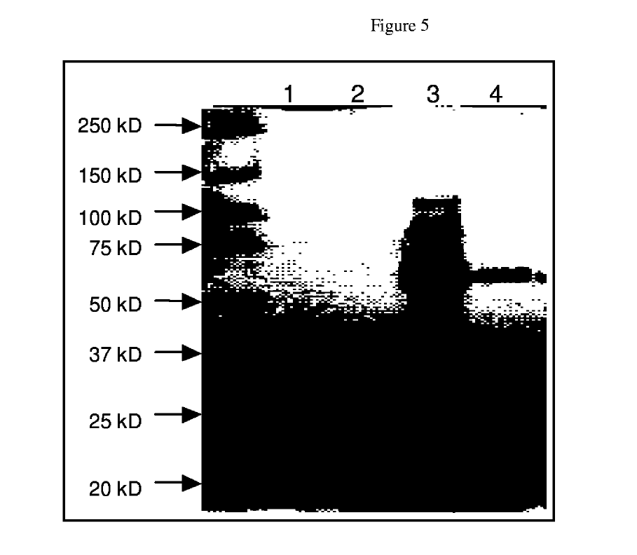

Infiltrated leaves were harvested after 72 hr and subjected to extraction and RTB purification based on anion exchange and lactose affinity chromatography (see Methods). Purity was assessed by observation of the proteins on silver stained 10% SDS-PAGE gels. There was little to no other bands seen, indicating a high level of purity. Plants infiltrated with empty-vector (pBIB-Kan) Agrobacterium and subjected to the exact same purification regime showed no bands on silver stain, indicating the specificity for RTB of this strategy. Identity of rRTB was confirmed by probing with RTB-specific antibodies, and by N-terminal sequencing. The level of expression of rRTB and RTB:GFP, as quantified via asialofetuin binding and Bradford assays was approximately 0.3% total soluble protein (TSP). The RTB-specific purification protocol typically produced 1-10 μg of purified protein per gram of leaf fresh weight. FIG. 5 is a silver stained 10% SDS-PAGE gel showing the purity of recombinant rRTB (lane 1), rRTB-fluorescein (lane 2), rRTB-biotin+fluorescein-streptavidin (lane 3), and RTB:GFP (lane 4).

Fluorescein Labeling and Biotinylation of cbRTB and rRTB

To ask if attaching small payload molecules to RTB at primary amine groups affected lectin activity, cbRTB and rRTB were labeled with NHS-fluorescein. N-hydroxysuccinimide (NHS) reagents react with exposed primary amine groups (at the N-terminus and lysine side-chains), to create an amide bond between the protein and the molecule to which NHS is esterified to, in this case fluorescein. RTB theoretically contains eight primary amines: one at the N-terminus, and seven lysine residues. Following the reaction and two rounds of dialysis against PBS, the fluorescein-labeled protein samples retained a distinct yellow color, indicative of successful labeling. cbRTB-fluorescein and rRTB-fluorescein produced a brilliant green color when subjected to UV light. The lectin binding activity of cbRTB-fluorescein and rRTB-fluorescein was tested via asialofetuin binding assay. These proteins did not exhibit significant reduction in binding capability compared to unlabeled RTB. These results suggest that labeling RTB through the primary amine groups does not interfere with lectin activity. In addition, there was no discernable difference in asialofetuin binding between rRTB and cbRTB, indicating that recombinant production of RTB does not impact its activity. SDS-PAGE analysis of rRTB-fluorescein shows a slightly slower mobility than unlabeled rRTB (FIG. 5, lanes 1 & 2). This difference in mobility is attributable to successful labeling of rRTB with NHS-fluorescein.

The streptavidin/biotin interaction was employed in order to determine if large proteins attached to RTB at primary amine groups interfere with lectin activity and cellular uptake. Streptavidin is a homo-tetramer with a molecular weight of about 67 kD. Each monomer binds one molecule of biotin, molecular weight 244.3 g/mol. This binding is one of the strongest known non-conjugated interactions, and it provides an easy way to attach a relatively large protein to RTB without creating a direct genetic fusion, by conjugating biotin to RTB then allowing streptavidin to bind the biotin. Biotinylation of cbRTB and rRTB was performed as described above using sulfo-NHS-LC-biotin, a water-soluble form of NHS-biotin that includes an 11 atom spacer arm (LC) between the NHS and the biotin, to reduce steric hindrances. The conjugation chemistry is the same as that described for the fluorescein labeling. A modified asialofetuin assay in which samples were probed with streptavidin-labeled horse radish peroxidase (HRP) was employed to assess the success of biotinylation. Comparing data obtained with this assay to an asialofetuin assay in which the same samples were probed with anti-ricin antibodies indicated that biotin was present on RTB, and that this RTB retained lectin activity. Unlabeled RTB, from both castor bean and recombinant sources and bound to asialofetuin, did not react with streptavidin-labeled HRP. Both cbRTB-biotin and rRTB-biotin bound to asialofetuin reacted strongly with streptavidin-HRP as visualized with HRP substrate at 450 nm. These findings show that cbRTB-biotin and rRTB-biotin retain both lectin activity and streptavidin binding ability. The extent of biotin labeling on a molar ratio was not investigated. cbRTB-biotin and rRTB-biotin was complexed with fluorescein-labeled streptavidin, (FITC-streptavidin; Sigma S-3762, dissolved in PBS) by mixing equal amounts of both proteins and incubating at room temperature for ˜20 min. As seen in FIG. 5 lane 3, SDS-PAGE analysis of rRTB-biotin/FITC-streptavidin complex shows several higher molecular weight bands indicative of the complex, as well as a light band corresponding to uncomplexed rRTB-biotin, which runs slower than rRTB. The gel running conditions undoubtedly break up some of the complex, as can be seen by the large ˜20 kD band corresponding to streptavidin monomers. As this sample did not undergo purification, and FITC-strep was in excess in the mixture, there was some FITC-strep that did not complex with rRTB-biotin, which appears as the ˜70 kD band.

### RTB Mediates Cell Uptake of Conjugated and Fused Payloads

Fluorescent microscopy is a powerful tool that allows investigation of several questions regarding RTB-mediated uptake. First, it allows visual observation of fluorescently-labeled RTB in real time to assess the impact of conjugation and fusion on cell uptake. Second, comparison of rRTB to cbRTB indicates the impact of recombinant versus native expression on uptake.

The assay employed to investigate answers to these questions utilized human epithelial HT-29 cells grown in a thin layer on the inner surface of a 96 well optical-bottom, black-walled microtiter plate. In preparation for sample loading, the cells were cooled by washing with ice-cold HBSS to slow the cell surface activity. Ice-cold samples containing RTB were applied and the plate was incubated at 4° C. to allow binding to the cell surface yet halting of endocytosis. After this incubation, the cells were washed again with ice-cold HBSS to wash away any unbound protein. Photographs taken at this T=0 time point usually show fluorescence only at the outer membrane, appearing as a complete outline of the cell. For control proteins such as GFP alone, no fluorescence is seen at T=0 indicating that binding was dependent on the RTB lectin activity. No internal structures are seen at this time. Incubation at 37° C. allows endocytotic processes to recommence, and uptake of the fluorescent-labeled protein was visualized in real time by taking photographs at specific time points (T=30, 60, and 120 min) following the shift to 37° C. If endocytosis and entry of labeled proteins into the endomembrane system occurs, this is typically observed as a reduced fluorescence at the plasma membrane and appearance of internal punctate fluorescence (presumably endosomes and lysosomes) at 60 and 120 min.

The various treatments tested in these experiments are listed in Table 6.

TABLE 6
Samples tested in HT-29 uptake assay

| | | HT-29 cell binding |
|---|---|---|
| Treatment # | Proteins tested | and uptake |
| 1 | cbRTB-fluorescein | + |
| 2 | rRTB-fluorescein | + |
| 3 | cbRTB + FITC-streptavidin | −− |
| 4 | cbRTB-biotin + FITC-streptavidin | + |
| 5 | rRTB-biotin + FITC-streptavidin | + |
| 6 | GFP | −− |
| 7 | RTB:GFP | + |

Approximately 500 ng of RTB equivalents (as determined by asialofetuin binding assay) of each treatment was applied to the cells in individual wells of an optical 96-well microtiter plates. Treatments 1 and 2 are fluorescein labeled cbRTB and rRTB, respectively and exhibited identical patterns of fluorescence as described above. Treatment 3 was non-biotinylated cbRTB, a negative control included to ensure that FITC-streptavidin was not capable of mediating uptake on its own. This is confirmed by absence of fluorescence at T=0. Treatments 4 and 5 were biotinylated cbRTB and rRTB, respectively, preincubated with FITC-streptavidin. Both showed positive fluorescence patterns over the time course, indicating the RTB mediated uptake of the fluorescent-labeled streptavidin. Treatment 6 was GFP alone, a negative control. Lack of fluorescence in treatment 6 indicated inability of GFP to bind to cell surfaces on its own. A positive fluorescence pattern in treatment 7, RTB:GFP, indicated RTB-mediated uptake of fused GFP. HT-29 cells stained only with LysoTracker-Red (Molecular Probes, Eugene Oreg.) show a very similar pattern, indicating that RTB locates primarily to endosomal and lysosomal compartments.

Discussion

We have reported the development of an Agrobacterium-mediated transient expression system using N. benthamiana to produce rRTB and RTB:GFP. Additionally we have shown that conjugation of proteins and small molecules to rRTB at primary amine groups does not adversely affect lectin activity and cellular uptake function. RTB has been expressed in other systems previously, such as E. coli²⁴, Saccaromyces cerevisiae²⁸, Xenopus oocytes²⁹, Spodoptera frugiperda (Sf9) cells^{30,31}, monkey kidney COS cells³², and tobacco²⁶. Additionally, ricin holotoxin has been produced in transgenic tobacco³³, and RTB:GFP has been produced in transgenic tobacco hairy root cultures.¹⁷ To our knowledge, RTB has not been produced in Nicotiana benthamiana using Agrobacterium-mediated transient expression. Work by Reed, et al. expressing hexahistidine-tagged RTB (6HIS-RTB) in stably-transformed tobacco reported an expression level of 0.007% TSP²⁶, in contrast to 0.3% TSP for rRTB in our system. Also, 6HIS-RTB transgenic lines produced at least three bands identified as RTB, due to alternative glycosylation forms and truncation at the N-terminus.²⁶ rRTB from our system was purified as a single band and possessed mannose-containing glycans (approximately 85% of a RTB-fusion bound to Concanavalin A-sepharose, data not shown).

Ricin-based vaccine strategies such as the work of Grimaldi, et al. require RTB to associate with antigen-fused RTA_{R180H}. The RTB used by this group is derived from castor bean, and therefore requires extensive purification to eliminate RTA and associated toxicity. This strategy has significant limitations for clinical applications both because of the potential of residual toxicity and the challenges of producing and processing highly toxic material at large scale. Absolute assurance of absence of RTA is guaranteed by producing RTB alone, recombinantly. In addition, the extensive tests involved in testing toxicity of payload-fused RTA_{R180H}:RTB conjugates is tedious and expensive.³⁴ It is doubtful that the public would accept a system based on RTB derived from ricin holotoxin, not to mention the costs such a system would incur to ensure safety and efficacy. rRTB produced in our system may in fact serve as a source for RTB in strategies such as that reported by Grimaldi et al. In our experiments, we did not attempt to associate rRTB with RTA to create ricin, as we do not have a source for the “disarmed” RTA_{R180H}. However, we are optimistic that our rRTB can successfully interact with RTA to form ricin. Furthermore, by producing rRTB and RTB-fusions in a plant system such as Agrobacterium-infiltrated N. benthamiana, all recovered RTB species are active and soluble, unlike rRTB produced in E. coli, which must be refolded with varying degrees of success.

The Agrobacterium-mediated transient expression of transgenes in host cells, and especially in plant cells is a very powerful tool. Many different genes and fusion constructs can be tested in a short amount of time compared to stable transformation of the same constructs. Additionally, the nature of the system is such that the expression profiles of infiltrated genes do not exhibit significant variation from plant-to-plant. By contrast, stable transformation of a single gene construct results in massive variations in expression levels among different plants transformed with the same construct, presumably due to so-called position effects, caused by the physical position of the transgene in the genome. The transient system utilizes a high Agrobacterium to plant cell ratio, and in contrast to stable transformation protocols, a higher number of inserted T-DNAs is desirable. The expression of the transgene is ectopic and transient in nature, and therefore the higher the number of T-DNAs to some saturation point, the greater the expression during the transient window. In stable transformation, one copy of inserted transgene per genome is desired, for silencing and genetics handling reasons. Both RTB:GFP and 6HIS-tagged rRTB are expressed at higher levels in transient systems compared to stable lines, presumably for these reasons.

The purification method employed, anion exchange on High Q followed by lactose affinity chromatography, proved to be very versatile in terms of time, effort, and specificity for a wide range of RTB-fusions. The regime allows for usable (μg-mg) amounts of rRTB or RTB-fusions to be extracted and purified in a single day. Using rRTB as a model, we estimate that our purification protocol yields ˜60% recovery. A broad range of diverse RTB-fusions, such as RTB:GFP, IL-12:RTB (see Liu & Cramer, 2006), RTB:κLC, and C_{L}:RTB have been purified using the exact same conditions. This flexibility enables rapid accumulation of purified RTB-fusion proteins, as specific purification conditions as influenced by each fusion partner may not have to be determined. This strategy complements the utility of the transient expression system and provides for a more user-friendly technology. In addition, this strategy is useful in investigating breakdown of certain RTB-fusions, as all breakdown products that contain an active RTB component are co-purified.

Conjugation of payloads to RTB at the primary amine groups did not significantly affect RTB-mediated binding to galactose or uptake by HT-29 cells. RTB contains eight possible sites of conjugation using this chemistry, at the N-terminus and seven lysine residues per polypeptide. Comparison of the bands in FIG. 5 lanes 1 and 2 indicate that labeling of the RTB by NHS-fluorescein is complete, however it is not apparent how many labels per RTB are present. The band in lane 2 is completely shifted upward compared to the band in lane 1. If a mixture of different ratio label-to-RTB species were present, one would expect to see a band in lane 2 that lined up with the bottom of the band in lane 1, but would be higher at the top. Instead, what is seen is that the bottom of the lane 2 band is higher than the bottom of the lane 1 band. This suggests that perhaps only one or perhaps a few different ratios of label-to-RTB species are present, which in turn suggests efficient labeling at all exposed sites. Uptake of FITC-streptavidin by biotinylated RTB showed that large proteins are able to be carried across the cell membrane in a RTB-mediated manner. The availability of primary amine groups on RTB suggest that ratios of FITC-streptavidin to biotinylated RTB may be greater than 1, indicating that RTB can deliver conjugated proteins at least 67 kD in size, and perhaps multiple copies of the same protein. In terms of usage as a vaccine delivery system, this method may be able to deliver up to eight copies of antigen per RTB molecule, which may drastically enhance the immunogenicity and efficiency of such a system. We have also demonstrated that hydrazine-derivatized molecules can be conjugated to oxidized sugars of the RTB N-linked glycans and both lectin binding and mammalian cell uptake activities were retained. Since RTB contains 2 N-linked glycan sites and each glycan contains multiple sugars, this strategy provides an additional route for attaching multiple payloads to RTB.

Direct fusion of RTB to payload, in this case GFP, showed that this method of delivery is also efficient. As mentioned earlier, visual observation of RTB:GFP uptake was not performed in earlier reports, and uptake was assumed as a prerequisite of immune response.¹⁷

### REFERENCES

1. Montfort, W., Villafranca, J. E., Monzingo, A. F., Ernst, S. R., Katzin, B., Rutenber, E., Xuong, N. H., Hamlin, R., & Robertus, J. D. The three-dimensional structure of ricin at 2.8 A. J. Biol. Chem. 262, 5398-5403 (1987).
2. Moisenovich, M., Tonevitsky, A., Maljuchenko, N., Kozlovskaya, N., Agapov, I., Volknandt, W., & Bereiter-Hahn, J. Endosomal ricin transport: involvement of Rab4- and Rab5-compartments. Histochem. Cell Biol. 121, 429-439 (2004).
3. Wesche, J. Retrograde transport of ricin. Int. J. Med. Microbiol. 291, 517-521 (2002).
4. Spooner, R. A., Watson, P. D., Marsden, C. J., Smith, D. C., Moore, K. A. H., Cook, J. P., Lord, J. M., & Roberts, L. M. Protein disulfide isomerase reduces ricin to its A and B chains in the endoplasmic reticulum. Biochem. J. 383, 285-293 (2004).
5. Wesche, J., Rapak, A., & Olsnes, S. Dependence of ricin toxicity on translocation of the toxin A-chain from the endoplasmic reticulum to the cytosol. J. Biol. Chem. 274, 34443-34449 (1999).
6. Deeks, E., Cook, J. P., Day, P. J., Smith, D. C., Roberts, L. M., & Lord, J. M. The low lysine content of ricin A chain reduces the risk of proteolytic degradation after translocation from the endoplasmic reticulum to the cytosol. Biochemistry 41, 3405-3413 (2002).
7. Olsnes, A. & Pihl, A. The molecular actions of toxins and viruses. (Elsevier Biomedical Press, New York; 1982).
8. Kreitman, R. J. & Pastan, I Immunotoxins in the treatment of hematologic malignancies. Curr. Drug Targets 7, 1301-1311 (2006).
9. Roth, R. A., Maddux, B. A., Wong, K. Y., Iwamoto, Y., & Goldfine, I. D. Insulin-ricin B chain conjugate: a hybrid molecule with ricin-binding activity and insulin biological activity. J. Biol. Chem. 256, 5350-5354 (1981).
10. Roth, R. A., Iwamoto, Y., Maddux, B. A., & Goldfine, I. D. Insulin-ricin B chain conjugate has enhanced biological activity in insulin-insensitive cells. Endocrinology 112, 2193-2199 (1983).
11. Hofmann, C. A., Lotan, R. M., Ku, W. W., & Oeltmann, T. N. Insulin-ricin B hybrid molecules mediate an insulin-associated effect on cells which do not bind insulin. J. Biol. Chem. 258, 11774-11779 (1983).
12. Hofmann, C. A., Lotan, R. M., Rath, G. D., & Oeltmann, T. N. Insulin-ricin B hybrid molecules: receptor binding and biological activity in a minimal-deviation hepatoma cell line. Arch. Biochem. Biophys. 227, 448-456 (1983).
13. Day, P. J., Ernst, S. R., Frankel, A. E., Monzingo, A. F., Pascal, J. M., Molina-Svinth, M. C., & Robertus, J. D. Structure and activity of an active site substitution of ricin A chain. Biochemistry 35, 11098-11103 (1996).
14. Beaumelle, B., Taupiac, M., Lord, J. M., & Roberts, L. M. Ricin A chain can transport unfolded dihydrofolate reductase into the cytosol. J. Biol. Chem. 272, 22097-22102 (1997).
15. Tagge, E., Harris, B., Burbage, C., Hall, P., Vesely, J., Willingham, M., & Frankel, A. Synthesis of green flourescent protein-ricin and monitoring of its intracellular trafficking. Bioconjugate Chem. 8, 743-750 (1997).
16. Grimaldi, E., Claassen, E. A. W., Lord, J. M., Smith, D. C., & Easton, A. J. Stimulation of pneumonvirus-specifc CD8+ T-cells using a non-toxic recombinant ricin delivery system. Mol. Immunol. 44, 993-998 (2007).
17. Medina-Bolivar, F., Wright, R., Funk, V., Sentz, D., Barroso, L., Wilkins, T. D., Petri, W., & Cramer, C. L. A non-toxic lectin for antigen delivery of plant-based mucosal vaccines. Vaccine 21, 997-1005 (2003).
18. Choi, N. W., Estes, M. K., & Langridge, W. H. R. Synthesis of a ricin toxin B subunit-rotavirus VP7 fusion protein in potato. Mol. Biotechnol. 32, 117-127 (2006).
19. Choi, N. W., Estes, M. K., & Langridge, W. H. R. Ricin toxin B subunit enhancement of rotavirus NSP4 immunogenicity in mice. Viral Immunol. 19, 54-63 (2006).
20. Becker, D. Binary vectors which allow the exchange of plant selectable markers and reporter genes. Nucl. Acid. Res. 18, 203-210 (1990).
21. Holsters, M., de Waele, D., Depicker, A., Messens, E., van Montagu, M., & Schell, J. Transfection and transformation of Agrobacterium tumefaciens. Mol. Gen. Genet. 163, 181-187 (1978).
22. Li, Y., Geng, Y., Song, H., Zheng, G., Huan, L., & Qiu, B. Expression of a human lactoferrin N-lobe in Nicotiana benthamiana with potato virus X-based agroinfection. Biotechnol. Lett. 26, 953-957 (2004).
23. Kapila, J., De Rycke, R., Van Montagu, M., & Angenon, G. An Agrobacterium-mediated transient gene expression system for intact leaves. Plant Sci. 122, 101-108 (1997).
24. Tonevitsky, A., Toptygin, A., Agapov, I., Pfüffer, U., & Frankel, A. Renaturated ricin B chain made in Escherichia coli is soluble, stable, and biologically active. Biochem. Mol. Biol. Int. 32, 1139-1146 (1994).
25. Choi, N., Estes, M. K., & Langridge, W. H. R. Mucosal immunization with a ricin toxin B-subunit-rotavirus NSP4 fusion protein stimulates a Th1 lymphocyte response. J. Biotechnol. 121, 272-283 (2006).
26. Reed, D. G., Nopo-Olazabal, L. H., Funk, V., Woffenden, B. J., Reidy, M. J., Dolan, M. C., Cramer, C. L., & Medina-Bolivar, F. Expression of functional hexahistidine-tagged ricin B in tobacco. Plant Cell Rep. 24, 15-24 (2005).
27. Frankel, A. E., Fu, T., Burbage, C., Tagge, E., Harris, B., Vesely, J., & Willingham, M. C. Lectin-deficient ricin toxin intoxicates cells bearing the D-mannose receptor. Carbohydrate Research 300, 251-258 (1997).
28. Richardson, P. T., Roberts, L. M., Gould, J. H., & Lord, J. M. The expression of functional ricin B-chain in Saccharomyces cerevisiae. Biochimca et Biophysica Acta 950, 385-394 (1988).
29. Wales, R., Richardson, P. T., Roberts, L. M., Woodland, H. R., & Lord, J. M. Mutational analysis of the galactose binding ability of recombinant ricin B chain. J. Biol. Chem. 266, 19172-19179 (1991).
30. Afrin, L. B., Gulick, H., Vesely, J., Willingham, M., & Frankel, A. E. Expression of oligohistidine-tagged ricin B chain in Spodoptera frugiperda. Bioconjug. Chem. 5, 539-546 (1994).
31. Ferrini, J. B., Martin, M., Taupiac, M. P., & Beaumelle, B. Expression of functional ricin B chain using the baculovirus system. Eur. J. Biochem. 233, 772-777 (1995).
32. Chang, M. S., Russell, D. W., Uhr, J. W., & Vitetta, E. S. Cloning and expression of recombinant, functional ricin B chain. Proc. Natl. Acad. Sci. 84, 5640-5644 (1987).
33. Sehnke, P. C. & Ferl, R. J. Processing of preproricin in transgenic tobacco. Protein Expression and Purification 15, 188-195 (1999).
34. Smith, D. C., Marsden, C. J., Lord, J. M., & Roberts, L. M. Expression, purification and characterization of ricin vectors used for exogenous antigen delivery into the MHC class I presentation pathway. Biol. Proced. Online 5, 13-19 (2003).

### Example 2

In Example 1 is demonstrated that attachment of small molecule and protein payloads to a recombinant ricin B-chain (rRTB) carrier through chemical conjugation at primary amine groups, biotin/streptavidin interactions or direct genetic fusion resulted in a flexible and efficient platform for delivery across outer cell membranes. The following experiment demonstrates improvement of the RTB-based carrier system by the development of a “capture and carry” coupling mechanism between carrier and payload. Ideally, one would want to attach payload proteins to the carrier through a breakable interaction. Allowing the payload and carrier to separate once inside the cell may give more flexibility and therefore a wider range of possible applications. Disulfide bonds are broken under very specific conditions, either through chemical reduction or enzyme mediated mechanisms. The major drawback of direct genetic fusion of carrier and payload proteins is the strength of the peptide bond, which usually requires a protease and an appropriate recognition sequence between fusion partners. Identifying the proper protease endogenous to the target cell that is located in the appropriate sub-cellular compartment would be tedious and may necessitate further research. Breakable interactions such as the disulfide bond may be engineered into the spacers integrated into chemical conjugation reagents, but systems involving chemical conjugation may be hampered by the cost of such a system on a large scale. Other drawbacks of chemical conjugation include difficulties presented by the specific chemistry of payload proteins (for example, lack of available conjugation sites) and by the lack of control on conjugation efficiency.

An effective and easy way to attach payload proteins to RTB via a disulfide bond is shown. Two exemplifications are provided to achieve such a coupling mechanism: Strategy I deals with exploitation of RTA structural domains involved in dimerization with RTB, and Strategy II involves the utilization of an immunoglobulin (Ig) heavy and light chain-based scaffolding platform to bridge carrier and payload proteins. Payload systems for each strategy were produced in both E. coli- and plant-based expression systems and evaluated for efficacy with a plant-derived RTB-carrier.

In designing experiments to evaluate Strategy I, it was first necessary to investigate the nature of the RTA:RTB interaction in ricin toxin more closely. Identification of RTA structural domains involved in interactions with RTB can be found in the work of Montfort, et al. and Rutenber & Robertus dealing with the three-dimensional crystal structures of ricin and RTB, respectively.^{1,2} In addition to the single disulfide bond between Cys 259 of RTA and Cys 4 of RTB, there are nine polar and six hydrophobic interactions between RTA and RTB. The strategy is to identify RTA structural domains that are sufficient to mediate interaction and disulfide linkage to RTB. Fusion of these domains to the C-terminus of a payload protein may mimic the RTA:RTB interaction, where the active and toxic portions of RTA have been effectively replaced with a beneficial payload protein.

The other system investigated as a means to connect payload to RTB carrier, termed here Strategy II, relied on the interaction between immunoglobulin (Ig) heavy- and light-chains (see FIG. 2). Ig light chains (LC) are composed of two domains, termed variable (V_{L}) and constant (C_{L}). Heavy chains (HC) are composed of one variable (V_{H}) and three (or more) constant domains (C_{H}1-C_{H}3). Domains V_{H} and C_{H}1 together are termed Fd. A single disulfide bond connects one HC to one LC at C_{H}1 and C_{L}. By fusing either LC or HC domains to RTB and the respective-interacting domain to payload proteins, is demonstrated one embodiment of a RTB-mediated “capture and carry” system.

Production of payloads in both E. coli and plants were investigated. Payloads produced in E. coli were purified and efficacy of interaction with a plant-derived RTB-carrier was assessed. One of the payload constructs used to test this strategy that was produced in E. coli contained a C-terminal tetracysteine (TC) motif, which has been shown to bind specifically to Lumio fluorescent reagents (Invitrogen, Carlsbad Calif.). Incorporation of the TC tag was thought to facilitate subsequent fluorescence microscopy of uptake in HT-29 cells. In the case of plant-derived payloads, the ability of plant cells to properly assemble separate HC and LC proteins to create a heterodimer was exploited. Rodríguez, et al. showed that when separate HC and LC gene cassettes (each gene is driven by separate promoter sequences) are placed on the same T-DNA, then transformed into tobacco by using an Agrobacterium-mediated system, full length antibodies were made.³ Hull, et al. has shown that individual strains of Agrobacterium, each harboring either HC or LC gene T-DNAs, can be mixed in equal volumes immediately prior to infiltration, and full-length, functional antibodies are produced.⁴ These studies prove that plant cells can synthesize correctly assembled Ig's from separate, co-infiltrated HC and LC genes The plant's ability to assemble these domains in our RTB carrier system, by genetic fusion of either a LC or HC domain to the RTB carrier and the corresponding HC or LC domain to the payload is demonstrated here.

### Methods

PCR Amplification of Gene Fragments for Constructs

Table 7 lists the various gene fragments used in these experiments, and primer sequences and templates used in PCR amplification reactions. PCR-generated fragments were gel purified (Qiagen, Valencia Calif.) and digested with the appropriate enzymes, then cleaned again before ligation. All constructs were first assembled in the pBC cloning vector (Stratagene La Jolla, Calif.). Following sequence confirmation, promoter:gene cassettes were subcloned into the pBIB-Kan⁵ binary vector as HindIII/SalI or HindIII/SacI fragments. The construct R6-2 has been described elsewhere⁶, and served as the source for both the dual enhanced cauliflower mosaic virus 35S promoter/tomato etch virus (TEV) translational enhancer/patatin signal peptide fragment (de35S:TEV::sp) as well as the XhoI-GFP-SacI fragment. All constructs used in plant-based expression incorporated the patatin signal peptide (sp) for targeting to the ER and secretion to the apoplast.⁷

TABLE 7
Substrates for PCR amplification of fragments used in gene construction

| | | |
|---|---|---|
| Fragment | Primer sequences† | Template |

| | | |
|---|---|---|
| NcoI-Fd-SalI | 5′-GATATACCCATGGTCCAGCT (F) | pET-Fd^{a} |
| | (SEQ ID NO: 11) | |
| | 5′-CTGACTGTCGACACAGATACCAGGGCAATTC (R) | |
| | (SEQ ID NO: 12) | |
| NcoI-Fd-stop-SalI | 5′-GATATACCCATGGTCCAGCT (F) | pET-Fd^{a} |
| | (SEQ ID NO: 11) | |
| | 5′-GTCGAC [symbol] ACAGATACCAGGGCAATTC (R) | |
| | (SEQ ID NO: 13) | |
| SacI-AdIII*-stop-XhoI | 5′-GAGCTCGCAGCAAGATTCCAATATATTG (F) | Preproricin^{b} |
| | (SEQ ID NO: 14) | |
| | 5′-CTCGAG [symbol] AAACTGTGACGATGGTG (R) | |
| | (SEQ ID NO: 15) | |
| XbaI-AdIII: RTB-stop-SalI | 5′-TCTAGACCAGATCCTAGCGTAATTAC (F) | Preproricin^{b} |
| | (SEQ ID NO: 16) | |
| | 5′-GTCGAC [symbol] AAATAATGGTAACCATA (R) | |
| | (SEQ ID NO: 10) | |
| XbaI-GFP-SacI | 5′-TCTAGAAGTAAAGGAGAAGAACTTTTCAC (F) | R6-2^{c} |
| | (SEQ ID NO: 17) | |
| | 5′-GAGCTCTTTGTATAGTTCATCCATGC (R) | |
| | (SEQ ID NO: 18) | |
| SacI-AdIII: RTB-stop-SalI | 5′-GAGCTCCCAGATCCTAGCGTAATTAC (F) | Preproricin^{b} |
| | (SEQ ID NO: 19) | |
| | 5′-GTCGAC [symbol] AAATAATGGTAACCATA (R) | |
| | (SEQ ID NO: 10) | |
| XbaI-RTB-stop-SalI | 5′-TCTAGAGCTGATGTTTCTATGGAT (F) | Preproricin^{b} |
| | (SEQ ID NO: 9) | |
| | 5′-GTCGAC [symbol] AAATAATGGTAACCATA (R) | |
| | (SEQ ID NO: 10) | |
| XbaI-RTB: linker-PstI | 5′-TCTAGAGCTGATGTTTCTATGGAT (F) | RTB: linker: GFP^{d} |
| | (SEQ ID NO: 9) | |
| | 5′-CTTTACTCATCTGCAGAGAACCTC (R) | |
| | (SEQ ID NO: 20) | |
| PstI-RTB-stop-SalI | 5′-CTGCAGGCTGATGTTTCTATGGATC (F) | Preproricin^{b} |
| | (SEQ ID NO: 21) | |
| | 5′-GTCGAC [symbol] AAATAATGGTAACCATA (R) | |
| | (SEQ ID NO: 10) | |
| PstI-C_{L}-stop-SalI | 5′-CTGCAGGATGCTGCACCAACTGTATC (F) | Mouse kappa LC^{e} |
| | (SEQ ID NO: 22) | |
| | 5′-GTCGAC [symbol] ACACTCATTCCTGTTGAAGC (R) | |
| | (SEQ ID NO: 23) | |
| XbaI-C_{L}-XhoI | 5′-TCTAGAGATGCTGCACCAACTGTATCC (F) | Mouse kappa LC^{e} |
| | (SEQ ID NO: 24) | |
| | 5′-CTCGAGACACTCATTCCTGTTGAAGCTC (R) | |
| | (SEQ ID NO: 25) | |
| PstI-C_{H}1-stop-SalI | 5′-CTGCAGAGAGAGCCCACCATCTAC (F) | Mouse alpha HC^{f} |
| | (SEQ ID NO: 26) | |
| | 5′-GTCGAC [symbol] ACAGATACCAGGGCAATTC (R) | |
| | (SEQ ID NO: 13) | |
| XbaI-C_{H}1-XhoI | 5′-TCTAGAAGAGAGCCCACCATCTACC (F) | Mouse alpha HC^{f} |
| | (SEQ ID NO: 27) | |
| | 5′-CTCGAGACAGATACCAGGGCAATTC (R) | |
| | (SEQ ID NO: 28) | |
| XbaI-Fd-XhoI | 5′-TCTAGAGTCCAGCTGCTCCAGTCT (F) | Mouse alpha HC^{f} |
| | (SEQ ID NO: 29) | |
| | 5′-CTCGAGACAGATACCAGGGCAATTC (R) | |
| | (SEQ ID NO: 28) | |
| EcoRI-Fd-stop-SalI | 5′-GAATTCGTCCAGCTGCTCCAGTCT (F) | Mouse alpha HC^{f} |
| | (SEQ ID NO: 30) | |
| | 5′-GTCGAC [symbol] ACAGATACCAGGGCAATTC (R) | |
| | (SEQ ID NO: 13) | |
| EcoRI-ιLC-stop-SalI | 5′-GAATTCGTTGTGATGACCCAAACTCCA (F) | Mouse alpha LC^{e} |
| | (SEQ ID NO: 31) | |
| | 5′-GTCGAC [symbol] ACACTCATTCCTGTTGAAGC (R) | |
| | (SEQ ID NO: 23) | |
| XbaI-Fd*-XhoI | 5′-TCTAGAGTCCAGCTGCTCCAGTCT (F) | Mouse alpha HC^{f} |
| | (SEQ ID NO: 29) | |
| | 5′-GATCCTCGAGGGAAGGTGGAGGAGG (R) | |
| | (SEQ ID NO: 31) | |

†Underlined sequences represent restriction sites used in cloning and bold sequences indicate stop codons incorporated into forward (F) and reverse (R) primers.
^{a}pET-Fd was created by cloning a NdeI-Fd-SalI fragment into pET41a, not used here. The forward primer used to make this construct was 5′-GACTCATATGGTCCAGCTGCTCCAGTCT (SEQ ID NO: 32). The reverse primer used was the same used for NcoI-Fd-SalI (see above).
^{b}Preproricin gene in pBI121, a gift of Dr. Sehnke.⁸
^{c}R6-2 is de35S:TEV::Pat:RTB:GFP in pBIB-kan, and is described elsewhere.⁶
^{d}RTB:linker:GFP was constructed by J. Liu (see Liu & Cramer, 2006).
^{e}Accession # BC002035.⁹
^{f}Accession # BC013490.⁹

### RTB-Carrier Constructs

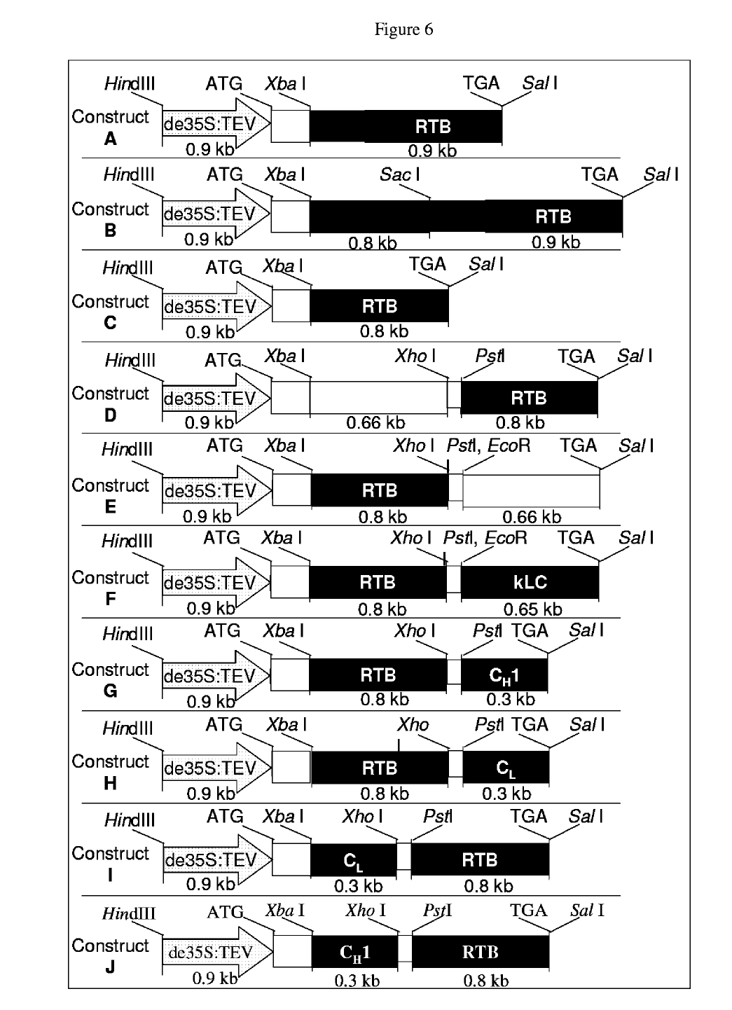

Maps of the constructs screened as potential RTB-carrier conformations used in both Strategies I and II can be seen in FIG. 6. The linker sequence used as a spacer between fusion partners was (Gly₃Ser)₃. All constructs were created in pBC or pBC-X cloning vectors. Promoter:gene cassettes were cloned into pBIB-Kan binary vector via HindIII/SalI or HindIII/SacI. The light shaded box appearing in constructs D-J represents the presence of the (Gly₃Ser)₃ linker peptide. Constructs A and B were used in RTA domain studies (Strategy I), constructs C through J were used in Ig scaffolding experiments (Strategy II). The positions of restriction sites utilized in cloning and start and stop codons are indicated.

In creating constructs in which RTB comprised the C-terminal partner, the XbaI-RTB:linker-PstI fragment (which contains a XhoI site between RTB and the linker) was first cloned into a modified pBC in which the XhoI site in the polylinker had been eliminated (pBC-X) via Mung bean nuclease digestion and re-ligation, a gift of Dr. Maureen Dolan (ABI), to give the plasmid pBC-X:RTB:linker. Plasmid pBC-X:RTB:linker was then digested with XbaI and XhoI to release a XbaI-RTB-XhoI fragment. Fragments such as XbaI-C_{L}-XhoI, XbaI-C_{H}1-XhoI, and XbaI-Fd-XhoI could then be ligated into the vector fragment to yield pBC-X:C_{L}:linker, etc. The PstI-RTB-SalI fragment was then ligated into these plasmids to yield fusions in which RTB was the C-terminal partner. In this way, all fusions between Ig domains and RTB contained the same linker sequence to maintain consistency. The GFP:AdIII:RTB construct includes both payload and carrier moieties, and is therefore included in FIG. 6. In constructs A and B, AdIII refers to amino acids 182 through 267 of RTA.

Construction of pET41-TC E. coli Expression Vector

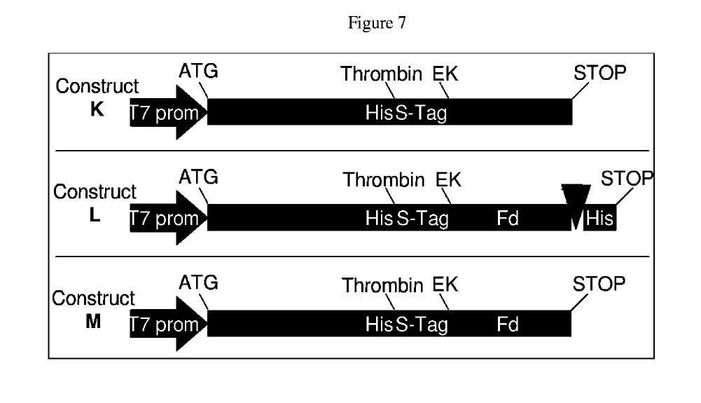

The pET41 (EMD Biosciences, San Diego Calif.) glutathione-S-transferase (GST) fusion vector for production of recombinant proteins in E. coli was modified to include the tetracysteine (TC) motif.¹⁰ The following oligonucleotides were annealed: 5′-CTGACTAAGCTTGAAGCTGGTGGCTGTTGTCCTGGCTGTTGCGGTGGCGGCACCGGTCTCGAGCTGACT (F) (SEQ ID NO: 33) and 5′-AGTCAGCTCGAGACCGGTGCCGCCACCGCAACAGCCAGGACAACAGCCACCAGCTTCAAGCTTAGTCAG (R) (SEQ ID No; 34). Underlined sequences indicate the TC motif (CCGPCC). The resulting fragment was digested with HindIII and XhoI and cloned into pET41b. The resulting plasmid, pET41-TC, contained the N-terminal GST tag, thrombin and enterokinase cleavage sites, a multiple cloning site for insertion of genes, the TC tag and a C-terminal 8HIS-tag (see FIG. 7, Construct L).

E. coli-Derived Payload Constructs

Maps of E. coli-produced payload constructs used in testing both RTA domain and HC:LC interaction strategies are shown in FIG. 7. Construct K was used in the assessment of the ability of the C-terminal domain III of RTA to mediate binding to RTB. Construct K was created by cloning the SacI-AdIII-stop-XhoI fragment into pET41a. Constructs L and M were tested as possible payloads utilizing the HC:LC scaffolding strategy. Construct L was created by cloning the NcoI-Fd-SalI fragment into pET41-TC, and construct M was created by cloning the NcoI-Fd-stop-SalI fragment into pET41a. In construct K, AdIII refers to amino acids 178-267 of RTA.

### Plant-Produced Payload Constructs

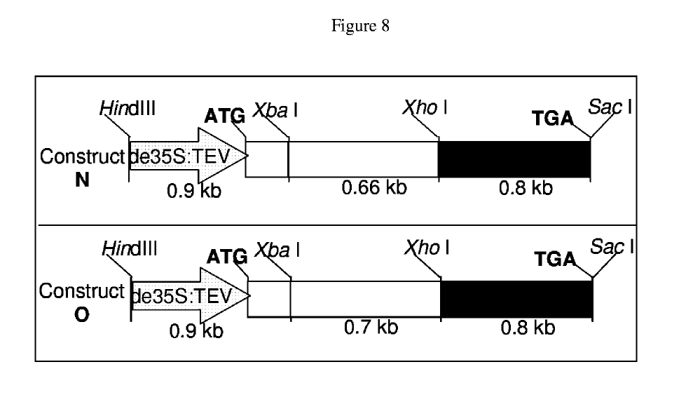

Constructs created for testing the plant cell's ability to assemble a HC:LC scaffolding between a co-expressed RTB-carrier and payload proteins (Strategy II) are depicted in FIG. 8. In testing the plant cell's ability to assemble carrier and payload in the RTA domain strategy, (Strategy I) constructs A and B were used. Both constructs N and O are pBIB-Kan-based plasmids and are driven by dual enhanced 35S CaMV promoter, and contain the TEV translational enhancer and sequences encoding the patatin signal peptide (sp). In construct O, “*” denotes the presence of sequences encoding the endogenous HC hinge region between Fd and GFP.

Agrobacterium tumefaciens-Mediated Transient Expression

pBIB-Kan plasmids harboring promoter:gene cassettes were transformed into A. tumefaciens strain LBA4404 using a modified freeze/thaw method.¹¹ Positive clones were grown in 50 mL YEP medium containing 100 μg/mL kanamycin and 60 μg/mL streptomycin for 48 hr at 28° C., 220 rpm. To induce A. tumefaciens prior to infiltration, cell pellets were harvested via centrifugation (5000×g for 10 min), resuspended in 300 mL induction media (20 mM MES pH 5.5, 0.3 g/L MgSO₄.7H₂O, 0.15 g/L KCl, 0.01 g/L CaCl₂, 0.0025 g/L FeSO₄.7H₂O, 2 mL/L 1 M NaH₂PO₄ pH 7.0, 10 g/L glucose) containing 100 μg/mL kanamycin and 60 μg/mL streptomycin, supplemented with 0.2 μM acetosyringone and incubated at 28° C., 220 rpm, for 4 hr to overnight. Induced A. tumefaciens cultures were introduced into four to six week old Nicotiana benthamiana plants either by pressure injection or vacuum infiltration. For pressure injection, a disposable syringe without a needle was filled with A. tumefaciens culture and pressed against the underside of the leaf.¹² For vacuum infiltration, plants were place upside-down in a beaker containing the induced culture so that all aerial portions were submerged. This was then placed inside a vacuum chamber and vacuum was applied (approximately 1 min) and broken by abruptly pulling off the tube from the chamber.¹³ This procedure was performed twice for each plant to ensure complete infiltration. Following infiltration, plants were returned to their growth chambers and allowed to incubate for 48-72 hr.

### Enrichment of RTB-Containing Fusion Proteins Using Immobilized Lactose

For initial characterization of RTB-containing transgenes products, infiltrated leaves were ground to a fine powder under liquid nitrogen (LN₂) using a mortar and pestle. Extraction buffer 1 (100 mM Tris, 100 mM Ascorbic acid, 150 mM NaCl, 20 mM EDTA, 2.5% PVPP) was added to the powder in a 2:1 volume buffer:mass leaf ratio. This crude extract was then centrifuged at 14,200×g for 30 min at 4° C. The supernatant was filtered through KimWipes to yield the cleared extract. Cleared extracts were batched with immobilized lactose resin (EY Laboratories, San Mateo Calif.) for 10-30 min at RT. Resin was collected by pouring the mixture into an empty disposable chromatography column and washed with 10 column volumes of PBS. RTB-containing fusion proteins were eluted by washing with 3×1 column volumes of 0.5 M D-galactose in PBS.

### Extraction and Purification of RTB-Containing Fusion Proteins

For experiments that required greater levels of purity, such as uptake studies, 10-20 g of infiltrated leaves were ground under LN₂ in a mortar and pestle to a fine powder. 50 mL of extraction buffer 2 (100 mM Tris-HCl pH 7.5, 20 mM D-galactose, 1% PVPP) was added to the powder and allowed to thaw at RT. The resulting crude extract was centrifuged at 14,200×g for 30 min at 4° C. The supernatant was filtered through KimWipes, brought to 100 mL with distilled H₂O and the pH was adjusted to 7.5 with 1 N NaOH. This cleared extract was then filtered though a 0.45 μm membrane and loaded onto an equilibrated 20 mL column volume MacroPrep High Q (Bio-Rad, Hercules Calif.) column using a Bio-Rad Duo-Flow FPLC system. Following loading of the sample, the column was washed with 80 mL of 50 mM Tris-HCl pH 7.5. The RTB-containing proteins were eluted and collected from the column by washing with 45 mL 50 mM Tris-HCl pH 7.5, 400 mM NaCl. The column was then cleaned by washing with 50 mM Tris-HCl pH 7.5, 1 M NaCl and re-equilibrated with 50 mM Tris-HCl pH 7.5. The RTB-containing sample (400 mM NaCl) was loaded onto a 1 mL immobilized lactose column (EY Laboratories, San Mateo Calif.) and washed with PBS. Purified RTB and RTB-containing fusion proteins were eluted by washing with 4×1 mL PBS+500 mM D-galactose. RTB-containing samples were then concentrated using YM-10 Centricons (Millipore Corp., Bedford Mass.) and dialyzed to PBS. Concentrated, dialyzed samples were then analyzed via Western blot using anti-RTB antibodies, silver stained SDS-PAGE, and asialofetuin binding assay.

Extraction and Purification of E. coli-Produced Payload Proteins

E. coli strain BL21(DE3) harboring Construct K (GST:AdIII) was grown in a 5 mL overnight LB culture containing kanamycin (100 μg/mL). This 5 mL culture was used to induce a 1 L LB (+100 μg/mL kanamycin) culture. The 1 L culture was grown at 37° C. at 225 rpm for ˜3 hr or until the OD₆₀₀ reached ˜0.6. The culture was induced by the addition of isopropyl-beta-D-thiogalactopyranoside (IPTG) to a final concentration of 1 mM. After 3 hr of induction at 37° C., 225 rpm, the cell pellet was recovered by centrifugation and resuspended in 30 mL cell lysis buffer (50 mM Tris HCl pH 8.0, 150 mM NaCl, 0.1% Triton X-100, 5 mM MgSO₄, 1 mg/mL lysozyme, 10 μg/mL DNase 1 and 10 μg/mL RNase A) and quickly frozen in LN₂, then allowed to thaw at RT. Lysed extract was centrifuged for 30 min at 14,200×g at 4° C., and inclusion bodies were recovered. No detectable fusion protein was observed in the soluble fraction, based on immobilized reduced glutathione (GST-Bind) chromatography. Insoluble GST:AdIII was refolded by first dissolving the inclusion bodies in 6 M urea, 50 mM Tris HCl, pH 8.0, 5 mM DTT and then removing the urea through successive rounds of dialysis. Dialysis buffers for removal of urea omitted DTT but contained 2.5 mM cysteine and 0.5 mM cystine, a redox pair used in facilitating disulfide bond formation. Following two additional rounds of dialysis against 50 mM Tris HCl pH 8.0, 150 mM NaCl, 5 mM DTT, the sample was applied to 2 mL of GST-Bind resin (EMD Biosciences, San Diego Calif.). The resin was then washed with 20 mL 50 mM Tris HCl pH 8.0, 150 mM NaCl, 5 mM DTT, and bound GST:AdIII was eluted by washing 3×2 mL 50 mM Tris HCl pH 8.0, 25 mM reduced glutathione (glut_{red}).

For recovery of GST:Fd:TC and GST:Fd, cultures of E. coli strains BL21(DE3) harboring either Construct L (GST:Fd:TC) or Construct M (GST:Fd) were inoculated in the same way described above. After 3 hr of growth at 37° C., 225 rpm (or when the OD_{600 ˜0.6}), the cultures were cooled by incubating on ice 5-10 min, then IPTG was added to a final concentration of 0.5 mM. Cultures were induced for 4 hr at 25° C., 225 rpm, then cell pellets were harvested by centrifugation. The cells were lysed by resuspension in 30 mL lysis buffer and application of a freeze/thaw cycle (described above). Lysed extracts were cleared via centrifugation at 14,200×g, for 30 min at 4° C. Cleared extracts were applied over a 2 mL GST-Bind resin column, and the resin was washed with 20 mL 50 mM Tris HCl pH 7.5, 150 mM NaCl, 5 mM DTT. GST:Fd:TC or GST:Fd was eluted from the column by washing with 3×2 mL 50 mM Tris HCl pH 7.5, 25 mM glut_{red}. For cleavage of the purification tag using thrombin, washed GST-Bind resin containing bound protein was suspended in 2 mL 50 mM Tris HCl pH 7.5, 150 mM NaCl, 20 mM CaCl₂, 5 mM DTT, and 1 unit of thrombin protease was added. The reaction occurred at RT for 4 hr, and cleaved protein was recovered by collecting the liquid fraction of the mixture (the GST tag remained bound to the resin) and washing the resin with 50 mM Tris HCl pH 7.5, 150 mM NaCl, 5 mM DTT. Fractions were pooled and concentrated.

### Asialofetuin Binding Assay.

A functional ELISA utilizing asialofetuin instead of a capture antibody was employed to assess galactose-specific lectin activity and quantify rRTB and RTB-containing fusion proteins. Asialofetuin is a modified mammalian glycoprotein that contains galactose-terminated glycans (Sigma, St. Louis Mo.). Asialofetuin at 300 μg/mL in PBS was bound to the wells of an Immulon 4HBX plate for 1 hr at RT. The wells were then blocked with 3% BSA in PBS for 1 hr at RT. Castor bean-derived RTB (cbRTB; Vector Labs, Burlingame Calif.) was used for the standard curve, ranging from 1.95 to 250 ng/well in PBS+10 mM D-galactose. For asialofetuin binding, 100 μL of standards and samples were incubated at RT for 1 hr. The plate was then washed 3× with PBS (300 μL/well). Rabbit anti-Ricinus communis lectin antibody (Sigma R-1254), diluted to 1:4000 in blocking buffer was then added (200 μL/well) and allowed to incubate for 1 hr at RT. Wells were washed again and alkaline phosphatase-labeled goat anti-rabbit antibody (Bio-Rad, Hercules Calif.), diluted 1:4000 in blocking buffer, was added and allowed to incubate for 45 min at RT. The wells were washed a third time and alkaline phosphatase substrate (100 μL/well; Pierce, Rockford Ill.) was applied. After the color developed sufficiently (10-15 min), the reaction was stopped with the addition of 50 μL 2 N NaOH and the absorbance at 405 nm was read. The inverse of the absorbance at 405 nm was plotted vs. the inverse of the standard RTB/well to give a linear relationship. This equation was then used to estimate the amount of RTB in the samples in terms of RTB equivalents.

By probing with antibodies other than the rabbit anti-Ricinus communis lectin antibody used for quantification, it was possible to use this assay to determine carrier/payload interactions. For example, in experiments in which GFP served as the model payload, binding of RTB-carrier to the payload was assessed by applying the samples to asialofetuin-coated wells and probing with anti-GFP antibodies. By inclusion of the proper controls, a positive reaction in this scenario indicated that GFP must be associated with RTB in order to bind to asialofetuin. Probing the same sample with different antibodies (in different wells) allowed determination of what proteins were present in the sample that had lectin-positive activity.

### Cell Uptake and Fluorescence Microscopy

The methods used were those described in Example 1, supra.

### Results

### Section 1: Development of RTB-Carrier Proteins

Strategy I: Carriers Used to Evaluate RTA Domain-Mediated Interaction with Payload

Recombinant RTB (rRTB, product of Construct C) and castor bean-derived RTB (cbRTB) were used as the carrier portion of the system to study the possibility of utilizing an E. coli-produced payload. The aim was to determine if AdIII could mediate interaction with un-modified RTB. Results of these experiments are discussed below.

### Strategy II: Evaluation of Potential Carrier Constructs in Engineering an Ig Domain Scaffolding Between Carrier and Payload

The potential of using the strong interactions between immunoglobulin (Ig) light and heavy chains as part of an RTB capture and carry system (Strategy II) was also tested. To develop the RTB-carrier component for Strategy II, a suite of fusion constructs between various Ig HC and LC domains and RTB were constructed, incorporating many possible orientations with regard to N- and C-terminal partner (Constructs D through J, FIG. 6). The Ig domains used were C_{L} (LC constant region), the entire mouse kappa light chain (κLC), C_{H}1 (HC constant region 1), and the Fd portion of the mouse alpha heavy chain. All of these domains contribute to the disulfide bond between HC and LC in full-length Ig's. Table 8 lists the constructs tested, their ability to drive product accumulation in Agrobacterium-infiltrated leaves, and the quality of product.

TABLE 8
Evaluation of potential RTB-carrier constructs using Strategy II

| | | Product | |
|---|---|---|---|
| Construct | Fusion | recovery^{a} | Quality of product^{b} |
| D | Fd:RTB | ++ | Breakdown |
| E | RTB:Fd | − | n/a |
| F | RTB:κLC | + | Intact |
| G | RTB:C_{H}1 | ++ | Breakdown |
| H | RTB:C_{L} | ++ | Breakdown |
| I | C_{L}:RTB | +++ | Breakdown |
| J | C_{H}1:RTB | ++ | Breakdown |

^{a}Relative expression levels were assessed via asialofetuin binding assay.
^{b}Quality of product refers to relative amount of full-length fusion protein compared to breakdown product as visualized on anti-RTB western blots (see FIG. 7);
n/a: not applicable

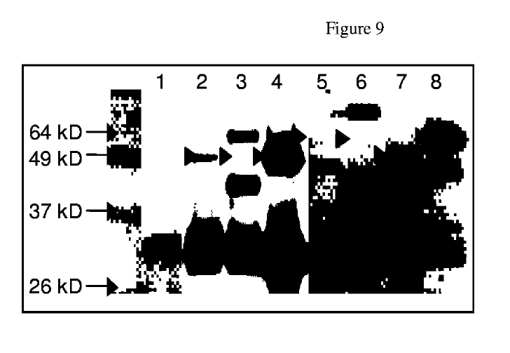

Constructs were assessed in regards to amount of full-length fusion protein (using predicted molecular weight estimates) via western immunoblot analysis using anti-RTB specific antibodies. FIG. 9 shows representative blots from these experiments. Constructs D through J were expressed in N. benthamiana leaves for 48 hours and the lactose-binding fraction was collected from leaf extracts. Samples were run on 12% reducing SDS-PAGE and transferred to nitrocellulose for Western blotting using anti-RTB antibodies. Two separate Western blots comprising all seven Strategy II carrier constructs were merged in this figure. The triangles indicate the predicted molecular weight for each constructs. Lane 1 is cbRTB, 30 ng; lane 2, RTB:C_{H}1 (Construct G); lane 3, C_{H}1:RTB; lane 4, C_{L}:RTB (I); lane 5, RTB:Rd (e); lane 6, Fd:RTB (d); lane 7, RTB:C_{L} (H); and lane 8, RTB:kLC (F). Constructs in which the fusion partner was on the N-terminus of RTB all showed a similar pattern of breakdown, with the major proportion of lactose-enriched protein migrating to the same position on SDS-PAGE as cbRTB and rRTB. Characterization of this breakdown and evidence for the presence of proteolytic-susceptible sites within RTB are presented in other examples here and will be discussed in much more detail there.

Of the seven constructs tested, only Construct F (RTB:κLC) generated a product that bound to lactose/asialofetuin, reacted with anti-RTB antibodies, and the majority of product matched the predicted molecular weight (˜55 kD) corresponding to the full-length fusion (FIG. 9, lane 8). However, RTB:κLC also had the lowest expression level among constructs which generated a product. Due to the observation that the vast majority of lectin-positive fusion was full length, RTB:κLC was chosen to move forward for payload capture studies.

### Development of RTB-Specific Purification Protocol

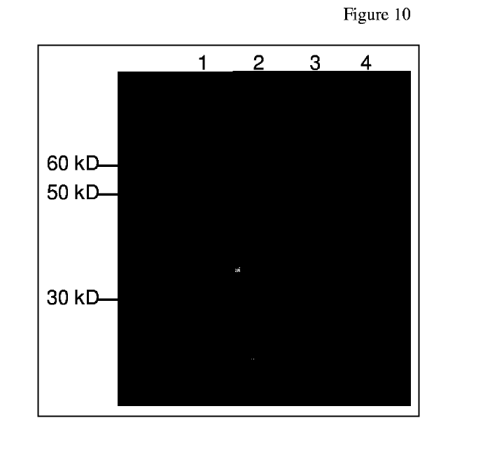

In order to achieve a higher level of purity of plant-produced RTB-carrier fusion proteins, a more rigorous purification regime was optimized. One-step chromatography using immobilized lactose enriched for proteins consisting of or containing a functional RTB unit, whether in the form of a full-length fusion or breakdown product, but many endogenous plant proteins co-purified. Many different chromatography supports were tested as a possible first step preceding lactose affinity, including cation exchange (both strong “S” and weak “CM”), anion exchange (strong “Q” and weak “DEAE”), hydroxyapatite, and hydrophobic interaction chromatography (following ammonium sulfate precipitation), to varying degrees of success (data not shown). Among these procedures, anion exchange resins appeared most effective in selectively enriching RTB and RTB-fusions. Utilizing the MacroPrep High Q resin (Bio-Rad, Hercules Calif.), It was possible to determine precise conditions in which RTB and RTB-fusions bound and eluted. The purification strategy used for purification of rRTB and all other RTB-fusions created thus far in our lab, in both the current research and others not discussed here, was developed using leaves expressing Construct I, C_{L}:RTB. This particular fusion was chosen for several reasons. First, the expression of asialofetuin-binding, anti-RTB-reactive proteins was relatively high with this construct, as determined by asialofetuin binding assays. Secondly, the presence of full-length product and two distinguishable breakdown bands allowed for developing conditions under which any RTB-containing protein could be purified using the same extraction buffer and elution conditions. The exact same protocol is used to purify a number of different RTB-containing fusion proteins, including RTB:κLC, RTB:GFP, rRTB, and IL12:RTB (See Liu & Cramer, 2006). All fusion partners tested purified using this protocol, indicating that the properties of RTB (overall charge, etc.) are being exploited, and not those of the fusion partner. FIG. 10 shows a silver stained SDS-PAGE gel of purified proteins. Lane 1 is C_{L}:RTB enriched via immobilized lactose affinity alone; lane 2, C_{L}:RTB purified via High Q followed by immobilized lactose affinity; lane 3, RTB:KLC purified by High Q and lactose affinity; and lane 4, empty-vector (pBK) Agro-infiltrated leaves subjected to High Q, immobilized lactose affinity chromatography. Note the difference in purity between C_{L}:RTB that has been enriched via lactose affinity alone (lane 1) and C_{L}:RTB that has undergone purification through anion exchange followed by lactose affinity (lane 2). The three bands seen in lane 2 all contain RTB, as determined through anti-RTB western blots (see FIG. 9, lane 4) and N-terminal sequencing of the two ˜30 kD bands. The high molecular weight band in lane 2 corresponds to full-length C_{L}:RTB (predicted size 42 kD). The one band at ˜55 kD in lane 3 is RTB:κLC. When leaves infiltrated with empty-vector A. tumefaciens are subjected to this purification regime, little to no proteins are visualized via silver stain (lane 4). This indicates that this purification regime is specific to RTB and RTB-containing fusion proteins.

### Strategy II: Characterization of RTB:κLC Carrier

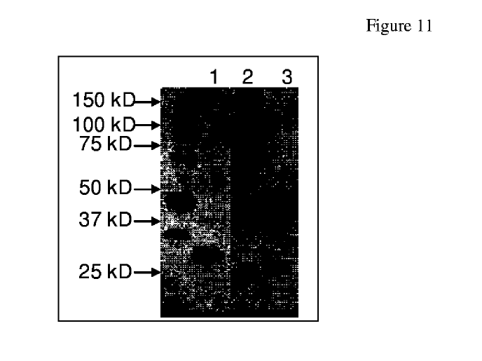

In order to assess whether RTB:κLC is of the proper conformation necessary for association with Fd, its natural partner (Fd), SDS-PAGE analysis under non-reducing and reducing conditions was performed. RTB:κLC exhibits slightly different migration patterns on SDS-PAGE under reducing and non-reducing conditions. This difference in migration can be seen in FIG. 11. Lane 1 is cbRTB (30 ng); lane 2, non-reduced RTB:κLC; and lane 3, reduced RTB:κLC. Non-reduced RTB:κLC runs slightly faster than reduced RTB:κLC, probably due to the presence of unbroken internal disulfide bonds that condense the structure of the fusion. There are four internal disulfide bonds in RTB and two in κLC. Reducing these bonds allows for an unrestrained, more open structure that exhibits a higher apparent molecular weight than non-reduced RTB:κLC. These data suggest that the plant cell is properly folding RTB:κLC.

Section 2: Evaluation of Assembly Between E. coli-Derived Payloads and RTB-Carrier
Strategy I: Refolding of E. coli-Produced Payload and Evaluation of Interaction with RTB Carrier

In order to test whether the C-terminal domain of RTA (AdIII) was sufficient to mediate interaction with RTB analogous to RTA:RTB association observed in ricin toxin, AdIII was fused to the C-terminus of glutathione-S-transferase (GST) by cloning the appropriate DNA fragment into pET41a (Construct K). Production of this fusion was problematic in that no significant levels of soluble protein were produced under a battery of different induction conditions (decreased induction temperatures and incubation times, lower IPTG concentrations, etc.). Therefore insoluble material was refolded by first dissolving the inclusion bodies in a buffer containing 6 M urea and through successive rounds of dialysis in the presence of the cysteine/cystine pair, the urea was removed by half each round. Following the complete removal of urea, the sample was dialyzed to conditions optimal for binding to immobilized reduced glutathione (glut_{red}). A large portion of refolded material bound and eluted from the GST-Bind resin, indicating successful refolding of the GST portion of the fusion protein.

To assess the ability of GST:AdIII to interact with RTB, two tests were performed. Refolded GST:AdIII was mixed with cbRTB in a 1:1 molar ratio in the presence of 1 mM DTT and dialyzed to PBS 2×. In the first test, the dialyzed mixture was applied to a modified asialofetuin assay that utilized anti-GST antibodies as the detection antibody. Successful interactions would be indicated by a positive reaction to the probe antibody. GST:AdIII bound directly to the plate (that is, no asialofetuin), was used as a control and reacted with the antibody. The samples applied to asialofetuin-coated wells did not react with anti-GST antibodies, but did react to anti-ricin antibodies (data not shown), indicating that binding between GST:AdIII and cbRTB did not occur. The second test involved inspection of migration patterns on SDS-PAGE under reducing and non-reducing conditions. A higher molecular weight band, at approximately the sum of the molecular weights of GST:AdIII (44 kD) and cbRTB (32 kD) should be present in non-reduced lanes if interactions did occur. Assembled products of this size was not observed. Thus, in contrast to E. coli-synthesized RTA,¹⁴ domain III alone does not appear effective in mediating in vitro assembly with RTB.

Strategy II: Expression and Purification of E. coli-Derived Payloads and Evaluation of Interaction with RTB:κLC

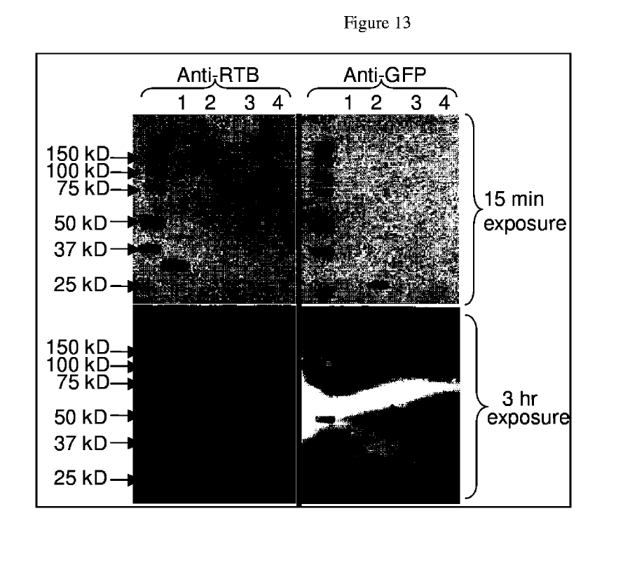

Since the selected Strategy II carrier is RTB:κLC, the corresponding payload must contain Fd, the portion of the HC that is composed of V_{H} and C_{H}1, the domains that interact with LC in immunoglobulins. The aim of these experiments was to determine if a Fd-containing payload protein produced in E. coli could bind to RTB:κLC produced in the plant expression system. Constructs L and M were expressed and the product was purified as described above. Construct L (GST:Fd:TC) contained the tetracysteine (TC) tag, a motif that preferentially binds a biarsenical fluorescent reagent, such as 4′,5′-bis(1,3,2-dithioarsolan-2-yl) fluorescein, marketed as Lumio Green® (Invitrogen, Carlsbad Calif.). This motif was included into the payload to facilitate downstream fluorescent microscopy of uptake experiments. GST:Fd:TC was expressed in a soluble form, and approximately 1.5 mg of purified protein was recovered per liter of culture, as determined by Bradford analysis of purified samples. To ensure that the GST tag did not interfere with binding to RTB:κLC, thrombin protease (EMD Biosciences, San Diego Calif.) was added to GST:Fd:TC bound to immobilized glut resin (GST-Bind). Digesting the protein with protease while immobilized allowed for complete removal of the tag, by freeing Fd:TC from the resin which is collected by washing with buffer. Subsequent elution of the resin-bound material revealed that virtually no intact GST:Fd:TC remained, as only a band matching the predicting molecular weight of GST was observed via Coomassie-stained SDS-PAGE (see FIG. 13; anti-RTB (left) and anti-GFP (right) Western analysis. Lane 1, cbRTB (30 ng); lane 2, GFP (30 ng); lane 3, Construct A, AdIII:RTB; lane 4, construct B, GFP:ADIII:RTB.).

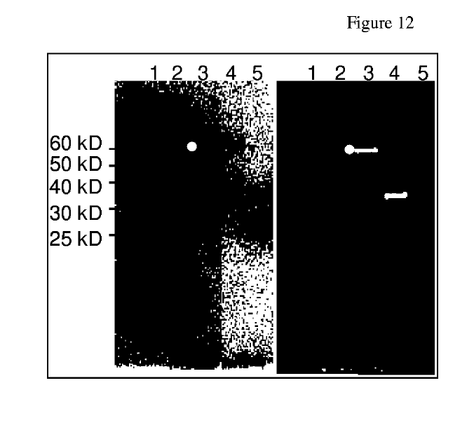

Addition of the Lumio Green® reagent to samples prior to gel-loading allowed for evaluation of the TC tag function. The gel was photographed on a standard UV light box set-up after running and prior to staining. Only proteins containing the TC tag and therefore the Lumio Green® reagent fluoresced when exposed to UV light (see FIG. 12: Lane 1: uninduced fraction; lane 2, induced soluble fraction; lane 3, purified full-length GST:Fd:TC (predicted molecular weight: 59 kD); lane 4, Fd:TC recovered thrombin digestion of immobilized glut_{red}-bound FST:Fd:TC (predicted molecular weight: 31 kD); lane 5, GST tag recovered after thrombin digest by washing resin with 25 mM glut_{red} (predicted molecular weight: 28 kD)). This type of analysis proved useful in expression and purification optimization studies.

To evaluate whether E. coli-produced Fd:TC could interact with plant-derived RTB:κLC, both proteins were purified, mixed in 1:1 molar ratios and dialyzed to PBS. Binding was assessed via a modified asialofetuin assay that used goat anti-mouse alpha chain probe antibody (Sigma A-4937). Fd:TC bound directly to the wells reacted well with this antibody, but no binding between RTB:κLC and Fd:TC was observed. Uptake studies using HT-29 cells also showed no binding (data not shown), both when the Lumio Green® reagent was added to the sample before applying to the cells, and when the Lumio Green® reagent was added to cells that had undergone incubation with the sample.

To determine if the TC or poly-histidine tags on the C-terminus of Fd:TC prevented binding to RTB:κLC, Construct M (GST:Fd) was developed, which included a stop codon at the 3′ end of the Fd gene. The expression and purification of GST:Fd was identical to that of Fd:TC. GST:Fd and Fd were labeled with NHS-fluorescein to facilitate fluorescence microscopy. No assembly between Fd and RTB:κLC was observed using the same tests as described above. Thus it appears that the immunoglobulin Fab domain can not be efficiently reconstituted in vitro using components independently produced in plants (RTB:κLC) and E. coli (heavy chain Fd and derivatives).

Section 3: Evaluation of Plant-Produced Payload Interactions with RTB-Carrier when Co-Expressed

The results described above suggest that the efficiency of in vitro assembly of components individually synthesized in plants (carrier) and bacteria (payload) is not sufficient to support capture and carry platforms based on either Strategy I (RTA-domain interactions with RTB) or Strategy II (Ig domain scaffolding). Strategies were therefore designed to assess the efficacy of these interactions under conditions where both components were co-synthesized in plant cells.

### Strategy I: RTA-Domain Mediated Interactions Between Payload and Carrier

In the case of plant-produced payload proteins in Strategy I, Construct B (GFP:AdIII:RTB) was employed. Construct B contains both carrier and payload in the same construct, and is therefore discussed here. The rationale behind Construct B was to replace the DNA encoding amino acids 1-181 of RTA with the gene encoding green fluorescent protein (GFP), as arranged in the native preproricin gene. The construct included the “proricin” 12 amino acid vacuolar-targeting linker. The idea was to see if the gene product of Construct B could mimic native ricin toxin processing, with the end result being GFP:AdIII bound to RTB via a disulfide bond between RTA Cys 259 (of AdIII) and RTB Cys 4 (see FIG. 1 for a theoretical representation). In these experiments, Construct A (AdIII:RTB) served as a control.

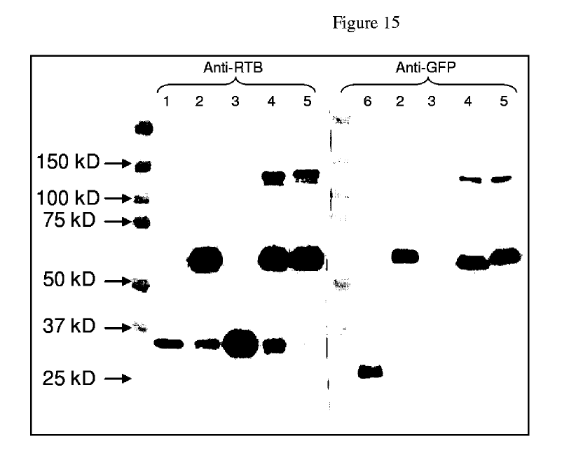

Constructs A and B were expressed using the Agrobacterium-mediated transient expression system. After 48 hr of incubation, RTB-containing proteins were enriched from leaf material using immobilized lactose chromatography. Elution fractions were separated on non-reducing 10% Tris-Glycine SDS-PAGE gels (Invitrogen) and transferred to nitrocellulose for western blot using anti-RTB or anti-GFP (CloneTech) specific antibodies. A ˜60 kD band that reacts with both anti-RTB and anti-GFP should be present from Construct B in order for this strategy to be successful. As seen in FIG. 13, no such band is present, even after a 3 hr exposure. In addition, very little ˜30 kD band representing RTB is present (seen in only the 3 hr exposure frame). In contrast, a strong cross-reacting band is typically observed with only 1-5 min exposure using constructs containing RTB:GFP in this system (see FIG. 15, lane 2). These results indicate that RTA domain III alone is insufficient to mediate interaction between GFP:AdIII and RTB. Furthermore, the absence of a strong band from Construct A indicates that these constructs may produce an unstable gene product.

### Strategy II: Assembly of Co-Expressed RTB:κLC Carrier and Fd:GFP Payload by Plant Cell

Since the carrier using Strategy II is RTB:κLC, the corresponding payload must contain the Fd portion of HC. Constructs consisting of Fd or Fd plus the HC hinge region fused to the N-terminus of GFP were prepared (Constructs N and O). In order to demonstrate effective and specific assembly of the RTB:κLC carrier with Fd-containing payloads, a variety of constructs were transiently expressed in leaves alone or in combination (see Table 9).

TABLE 9
Products used in evaluation of plant cell-mediated assembly of
RTB:κLC carrier and Fd:GFP payload

| | | | | Expected size of |
|---|---|---|---|---|
| | | | Individual | assembled |
| Treatment ID | Constructs Infiltrated | Gene Products | expected size | products |
| I | pBIB-Kan (empty | n/a | n/a | n/a |
| | vector) | | | |
| II | R6-2^{a} | RTB:GFP | 60 kD | n/a |
| III | F & N^{b} | RTB:κLC & | 55 kD & 51 kD | 106 kD |
| | | Fd:GFP | | |
| IV | F & O^{b} | RTB:κLC & | 55 kD & 52 kD | 107 kD |
| | | Fd*:GFP | | |
| V | C & N^{b} | rRTB & Fd:GFP | 32 kD & 51 kD | n/a |
| VI | C & O^{b} | rRTB & Fd*:GFP | 32 kD & 52 kD | n/a |

^{a}see Medina-Bolivar, et al.⁶
^{b}Two constructs were simultaneously infiltrated into the same plant by mixing equal volumes of induced A. tumefaciens cultures immediately prior to infiltration.

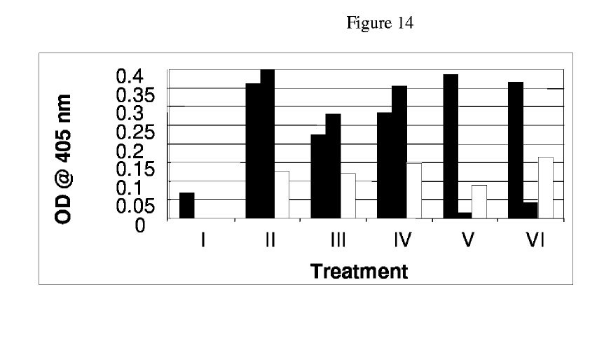

Crude leaf extracts generated from the treatments listed in Table 9 were tested for the presence of interactions between RTB and GFP. FIG. 14 shows the results of these assays. Crude extracts of the various treatments were tested on asialofetuin probed with anti-ricin (far left bar, light shading), asialofetuin probed with anti-GFP (middle bar, dark shading) and standard GFP ELISA (far right bar, no shading). All treatments except empty-vector control (treatment I) gave positive responses when bound to asialofetuin and probed with anti-ricin antibodies, indicating the presence of RTB-containing proteins in the crude extracts of treatments II through VI. A standard sandwich GFP ELISA was also performed, in which wells were coated with monoclonal anti-GFP specific antibodies and probed with polyclonal (rabbit) anti-GFP. GFP ELISA data confined the expression of GFP-containing constructs by the plant in treatments II through VI. Demonstration of a κLC:Fd scaffold bridging RTB to GFP was accomplished by comparing the responses of asialofetuin-bound samples probed with both anti-ricin and anti-GFP specific antibodies (both developed in rabbit), in separate wells (see FIG. 14). Only RTB:GFP (treatment II) and RTB:κLC co-expressed with either Fd:GFP (treatment III) or Fd*:GFP (treatment IV) gave positive reactions when asialofetuin-bound samples were probed with anti-GFP antibodies. These data indicate that the assembly of RTB-carrier and GFP-payload by the plant cell is mediated by κLC:Fd interactions.

In order to assess the ability of κLC:Fd interactions to mediate co-purification of carrier and payload, selected treatments were subjected to the standard RTB-specific purification protocol described above and analyzed via anti-RTB western blots (FIG. 15). (Lane 1: cbRTB (30 ng; lane 2, RTB:GFP (Treatment II); lane 3, rRTB and Fd:GFP (Treatment V); lane 4, RTB: KLC and Fd:GFP (Treatment III); lane 5, RTB:κLC and Fd*:GFP (Treatment IV); lane 6, GFP 30 ng.) The predicted molecular weight of RTB: GFP is 60 kD; RTB: KLC is 55 kD, rRTB is 32 kD, Fd:GFP is 51 kD, Fd* is 52 kD and GFP is 28 kD. As shown in FIG. 15, lanes 4 and 5, RTB-purified samples from leaves co-expressing RTB:κLC with either Fd:GFP (treatment III) or Fd*:GFP (treatment IV) showed bands of the expected sizes using both anti-RTB and anti-GFP detection antibodies. In contrast, no GFP was recovered from purified fractions from leaves co-expressing rRTB and Fd:GFP (FIG. 15, lane 3). The higher molecular weight band seen in FIG. 15, lanes 4 and 5 of both immunoblots is consistent with the size expected of the expected dimer (˜110 kD) and suggest that the samples may not have been fully reduced. RTB:GFP (treatment II) served as a positive control in these experiments. These data show that Fd:GFP co-purifies with RTB:κLC, but not rRTB, using a RTB-specific purification regime. These findings are consistent with κLC:Fd scaffolding bridging RTB-carrier and GFP-payload.

This two component system based on Ig domain scaffolding was designed to mimic RTA:RTB interactions, which involve a single intermolecular sulfhydryl bridge that is reduced in the ER of mammalian target cells permitting dissociation. In order to test whether assembled product from RTB:κLC and Fd: GFP co-expressed in plant cells was sulfhydryl-linked, electrophoretic mobility in the presence and absence of reducing agent (DTT) was compared. Purified protein from treatment IV was run on 10% SDS-PAGE under both reducing and non-reducing conditions and both silver stained and transferred to nitrocellulose for anti-RTB western analysis.

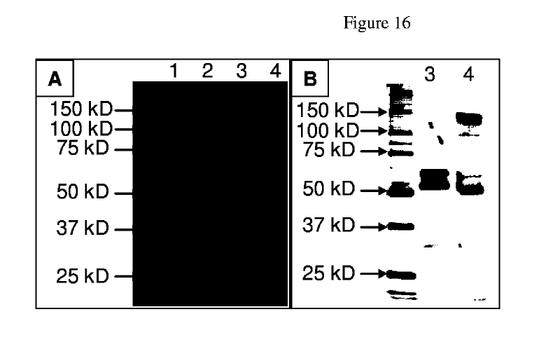

As shown in FIG. 16, RTB:κLC and Fd*:GFP in the presence of reductant migrate to their respective molecular weights. FIG. 16A is anti-RTB Western blot of RTB-purified samples, with lane 1, cbRTB 30 ng; lane 2, RTB:GFP; lane 3, RTB: κLC and Fd*:GFP reduced; lane 4, RTB:κLC and Fd*:GFP non-reduced. FIG. 16B is a Western of purified RTB: LC and Fd*:GFP samples, lane 3, RTB: κLC and Fd*:GFP reduced, lane 4, RTB: κLC and Fd*:GFP non-reduced. Under non-reducing conditions, the proteins migrate much slower, corresponding to a RTB:κLC and Fd*:GFP heterodimer (lanes 4). These data indicate that RTB:κLC and Fd*:GFP are held together via disulfide bonding.

### Assessment of Uptake Function of Plant-Produced Strategy II Carrier/Payload System

Mammalian cell uptake studies were used to assess the ability of the RTB:κLC carrier to successfully deliver its model payload Fd:GFP, in addition to mediating co-purification of co-expressed proteins, in order to show that κLC:Fd serves as a scaffolding to link a payload protein to RTB-carrier. Purified proteins from treatments II, III, IV and V were applied to HT-29 cells and uptake was assessed by fluorescence microscopy at 30, 60, and 120 min after binding (see Methods). rRTB labeled with NHS-fluorescein (rRTB-fl) and RTB:GFP served as positive controls. Fluorescence microscopy using a GFP filter showed that RTB:κLC, but not rRTB, mediated uptake of Fd:GFP. This is consistent with the analyses described above which showed that Fd:GFP co-purifies with RTB:κLC but not rRTB when co-expressed in plant leaves. At T=0, cell surface-bound GFP is clearly visible in the positive controls and protein from treatments III and IV. By T=60, clear punctate structures are observed which increase by T=120, indicating internalization and accumulation in endosomal and/or lysosomal compartments. No fluorescence was observed at any time point in rRTB+Fd:GFP samples. GFP alone (Clontech) applied to cells as a negative control likewise showed no binding or uptake into HT-29 cells.

Discussion

A modular, “plug and play” RTB-mediated protein delivery system has been developed. Of the two strategies tested, exploitation of RTA-domains to mediate interaction with RTB (Strategy I) and utilization of an Ig domain scaffold to bridge carrier and payload (Strategy II), only Strategy II implemented in a plant-based production system produced the flexibility and efficiency desired for this technology. By taking advantage of the plant cell's ability to assemble co-expressed κLC- and Fd-containing fusion proteins, it is shown that RTB:κLC can capture payload proteins (in this case Fd:GFP) co-expressed in the same cell, facilitate purification using a RTB-specific protocol, and mediate uptake in human epithelial cells. Additionally, it is shown that the interaction between carrier and payload includes a disulfide bond, which may allow for dissociation of carrier and payload once internalized by the target cell.

Plants are well suited for production of a modular system of this type. The Agrobacterium-mediated transient expression allows for great flexibility in terms of the number of different genes which can be co-expressed in a single plant, by simply mixing different Agrobacterium strains immediately prior to infiltration.

Payload proteins produced in E. coli for test strategies did not bind to their respective RTB-carriers. In the case of Strategy I, this may be due to the fact that the payload underwent a refolding process that, while restoring activity to the GST portion of the fusion, may not have been efficient in producing a properly refolded AdIII moiety. However, subsequent to the initiation of this strategy, a report was published¹⁵ indicating the lack of RTA His 40 is more likely to be responsible for the failure of GST:AdIII to associate with RTB. Of the sixteen interactions between RTA and RTB mentioned earlier, only His 40 and Glu 41 do not reside within the last 85 amino acids of RTA. His 40 and Glu 41 of RTA form the bend point for a loop that interacts with Asp 94 and Lys 219 of RTB, respectively. His 40 of RTA and Asp 94 of RTB, in addition to this interaction, also make up two of the three residues that form a recently discovered lipase active site (the third residue is Ser 221 of RTA). When researching this lipase activity, Morlon-Guyot, et al. discovered that when His 40 of RTA was mutated to Ala, the resulting protein associated only poorly with RTB, confirming the importance of this interaction.¹⁵ This idea is bolstered by the failure of the GFP:AdIII:RTB construct, produced in plants, to mimic the natural processing of preproricin. The E. coli-produced proteins for use in evaluation Strategy II, GST:Fd:TC, Fd:TC, GST:Fd, and Fd did not assemble with RTB:κLC. This observation reinforces the notion that assembly of κLC and Fd into a heterodimer relies on co-expression of κLC and Fd genes and possibly chaperone-mediated assembly within the same cell, as immunoglobulins are naturally produced. The inventors are not aware of any reports in which antibodies or Fab fragments have be assembled from independently synthesized and purified HC and LC proteins.

Linking a payload to the RTB-carrier by using this Ig scaffolding has benefits for certain applications over chemical conjugation and direct genetic fusion. Allowing the plant cell to assemble separate, co-expressed carrier and payload molecules precludes the need to perform unpredictable and possibly inefficient chemical conjugation chemistries. Direct genetic fusion of payload to RTB produces numerous concerns. First, the stability of different RTB-payload fusions varies greatly. Secondly, direct fusion, without incorporation of a proteolytic recognition site, is generally unbreakable and therefore the payload is obliged to traffic to which ever compartment RTB goes to when endocytosed. For example, vaccine antigens designed for presentation via the MHC class I pathway may remained trapped within the endosomal and/or only presented via MHC class II pathways. A disulfide bond may be broken through changes in reduction potential and/or enzymatic activity, and perhaps by including sub-cellular localization signals on the payload it may be possible to direct carrier and payload to different compartments upon internalization.

Possible applications of this system include vaccines and enzyme replacement therapy (ERT). RTB or ricin is useful to facilitate immune response to antigen proteins. ERT, especially in the area of lysosomal storage disorders, is an attractive candidate application because of the characteristics of RTB trafficking upon endocytosis in target cells. Only a small fraction of internalized RTB/ricin moves through the retrograde ER pathway, while the majority moves to endosomal and lysosomal compartments.¹⁶ Exploiting this natural tendency of RTB may prove very effective in delivery of lysosomal proteins such as glucocerebrosidase and iduronidase. Current ERT strategies rely on in vitro manipulation to provide the presence of mannose and mannose-6-phosphate glycans on the enzymes (to mediate uptake via cell-surface mannose receptors), and new advances in this strategy have been few. Perhaps combining the lectin-mediated uptake capabilities of RTB with the endogenous glycans of both RTB and the payload will improve the efficiency of ERT and result in lower cost and increased efficacy.

### REFERENCES

1. Montfort, W., Villafranca, J. E., Monzingo, A. F., Ernst, S. R., Katzin, B., Rutenber, E., Xuong, N. H., Hamlin, R., & Robertus, J. D. The three-dimensional structure of ricin at 2.8 A. J. Biol. Chem. 262, 5398-5403 (1987).
2. Rutenber, E. & Robertus, J. D. Structure of Ricin B-Chain at 2.5 A Resolution. Proteins 10, 260-269 (1991).
3. Rodríguez, M., Ramírez, N. I., Ayala, M., Freyre, F., Pérez, L., Triguero, A., Mateo, C., Selman-Housein, G., Gavilondo, J. V., & Pujol, M. Transient expression in tobacco leaves of an aglycosylated recombinant antibody against the epidermal growth factor receptor. Biotechnol. Bioeng. 89, 188-194 (2005).
4. Hull, A. K., Criscuolo, C. J., Mett, V., Groen, H., Steeman, W., Westra, H., Chapman, G., Legutki, B., Baillie, L., & Yusibov, V. Human-derived, plant-produced monoclonal antibody for the treatment of anthrax. Vaccine 23, 2082-2086 (2005).
5. Becker, D. Binary vectors which allow the exchange of plant selectable markers and reporter genes. Nucl. Acid. Res. 18, 203-210 (1990).
6. Medina-Bolivar, F., Wright, R., Funk, V., Sentz, D., Barroso, L., Wilkins, T. D., Petri, W., & Cramer, C. L. A non-toxic lectin for antigen delivery of plant-based mucosal vaccines. Vaccine 21, 997-1005 (2003).
7. Iturriaga, G., Jefferson, R. A., & Bevan, M. W. Endoplasmic reticulum targeting and glycosylation of hybrid proteins in transgenic tobacco. The Plant Cell 1, 381-390 (1989).
8. Sehnke, P. C. & Ferl, R. J. Processing of preproricin in transgenic tobacco. Protein Expression and Purification 15, 188-195 (1999).
9. Strausberg, R. L., Feingold, E. A., Grouse, L. H., Derge, J. G., Klausner, R. D., Collins, F. S., Wagner, L., Shenmen, C. M., Schuler, G. D., Altschul, S. F., Zeeberg, B., Buetow, K. H., Schaefer, C. F., Bhat, N. K., Hopkins, R. F., Jordan, H., Moore, T., Max, S. I., Wang, J., Hsieh, F., Diatchenko, L., Marusina, K., Farmer, A. A., Rubin, G. M., Hong, L., Stapleton, M., Soares, M. B., Bonaldo, M. F., Casavant, T. L., Scheetz, T. E., Brownstein, M. J., Usdin, T. B., Toshiyuki, S., Carninci, P., Prange, C., Raha, S. S., Loquellano, N. A., Peters, G. J., Abramson, R. D., Mullahy, S. J., Bosak, S. A., McEwan, P. J., McKernan, K. J., Malek, J. A., Gunaratne, P. H., Richards, S., Worley, K. C., Hale, S., Garcia, A. M., Gay, L. J., Hulyk, S. W., Villalon, D. K., Muzny, D. M., Sodergren, E. J., Lu, X., Gibbs, R. A., Fahey, J., Helton, E., Ketteman, M., Madan, A., Rodrigues, S., Sanchez, A., Whiting, M., Madan, A., Young, A. C., Shevchenko, Y., Bouffard, G. G., Blakesley, R. W., Touchman, J. W., Green, E. D., Dickson, M. C., Rodriguez, A. C., Grimwood, J., Schmutz, J., Myers, R. M., Butterfield, Y. S., Krzywinski, M. I., Skalska, U., Smailus, D. E., Schnerch, A., Schein, J. E., Jones, S. J., & Marra, M. A. Generation and initial analysis of more than 15,000 full-length human and mouse cDNA sequences. Proc. Natl. Acad. Sci. U.S. A 99, 16899-16903 (2002).
10. Griffin, B. A., Adams, S. R., & Tsien, R. Y. Specific covalent labeling of recombinant protein molecules inside live cells. Science 281, 269-272 (1998).
11. Holsters, M., de Waele, D., Depicker, A., Messens, E., van Montagu, M., & Schell, J. Transfection and transformation of Agrobacterium tumefaciens. Mol. Gen. Genet. 163, 181-187 (1978).
12. Li, Y., Geng, Y., Song, H., Zheng, G., Huan, L., & Qiu, B. Expression of a human lactoferrin N-lobe in Nicotiana benthamiana with potato virus X-based agroinfection. Biotechnol. Lett. 26, 953-957 (2004).
13. Kapila, J., De Rycke, R., Van Montagu, M., & Angenon, G. An Agrobacterium-mediated transient gene expression system for intact leaves. Plant Sci. 122, 101-108 (1997).
14. Smith, D. C., Marsden, C. J., Lord, J. M., & Roberts, L. M. Expression, purification and characterization of ricin vectors used for exogenous antigen delivery into the MHC class I presentation pathway. Biol. Proced. Online 5, 13-19 (2003).
15. Morlon-Guyott, J., Helmy, M. E., Lombard-Frasca, S., Pignol, D., Piéroni, G., & Beaumelle, B. Identification of the ricin lipase site and implication in cytotoxicity. J. Biol. Chem. 278, 17006-17011 (2003).
16. Moisenovich, M., Tonevitsky, A., Maljuchenko, N., Kozlovskaya, N., Agapov, I., Volknandt, W., & Bereiter-Hahn, J. Endosomal ricin transport: involvement of Rab4- and Rab5-compartments. Histochem. Cell Biol. 121, 429-439 (2004).
17. Marillonnet, S., Thoeringer, C., Kandzia, R., Klimyuk, V., & Gleba, Y. Systemic Agrobacterium tumefaciens-mediated transfection of viral replicons for efficient transient expression in plants. Nat. Biotechnol. 23, 718-723 (2005).

### Example 3

In the course of experiments aiming to engineer the ricin B lectin (RTB) into a flexible and efficient platform for protein delivery, a pattern of breakdown of RTB-fusion proteins recombinantly produced in Nicotiana benthamiana emerged. This breakdown was also observed with RTB-fusions produced in stably transformed Nicotiana tobacum suggesting it is not specific to either the transient expression system or to N. benthamiana. Fusions in which RTB constituted the C-terminal partner exhibited a near-predictable pattern, in which the large majority of lactose-binding product migrated to the same position on SDS-PAGE analysis as RTB from castor bean (cbRTB) and rRTB (˜32 kD). Only a fraction of recovered recombinant protein was of the predicted molecular weight, that being the sum of the N-terminal fusion partner plus RTB. This pattern of breakdown was observed regardless of the nature of the N-terminal fusion partner; proteins as diverse as immunoglobulin (Ig) domains, interleukins, and storage protein domain (SPD), when fused to the N-terminus of RTB displayed this pattern. Fusions in which RTB constituted the N-terminus, such as RTB:GFP and RTB:κLC, did not display this phenomenon as obviously as when RTB was on the C-terminus. Clearly there was something about RTB, and not the fusion partner, that was mediating this breakdown.

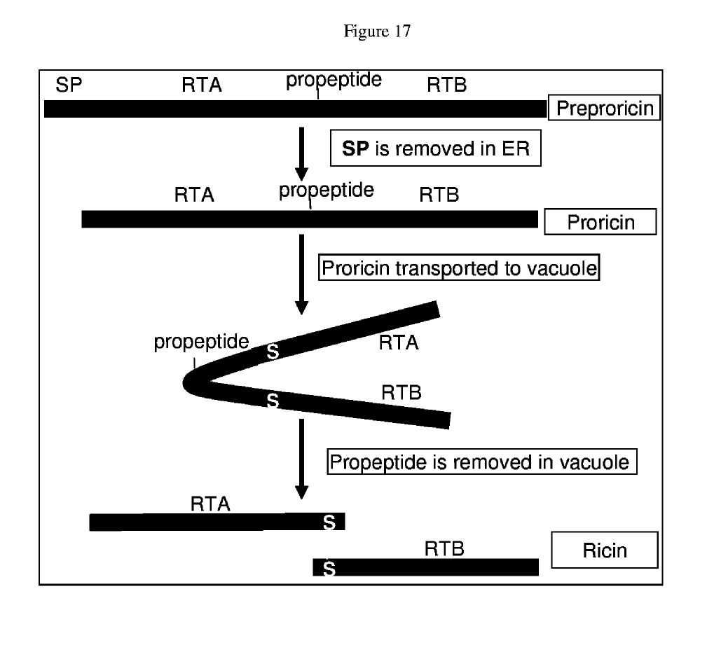

Clues as to the possible origin of this phenomenon may be found in the post-translational processing of ricin toxin in Castor bean seeds. FIG. 17 illustrates this processing. The primary gene product of the ricin toxin gene is a polypeptide chain termed preproricin.¹ Preproricin consists of the N-terminal endoplasmic reticulum (ER)-targeting signal peptide, the A-chain N-glycosidase toxin (RTA), a short 12 amino acid linker sequence which contains vacuolar sorting information², and RTB. Following insertion into the ER and subsequent cleavage of the signal peptide, preproricin becomes proricin: RTA fused to RTB through the 12 amino acid vacuolar-targeting linker. It is not known precisely when this linker sequence is removed completely, but it is thought to occur once in the vacuole. Following removal of the linker, separated RTA and RTB polypeptide chains are held together via a single disulfide bond and numerous hydrophobic and polar interactions.^{3,4} The molecular weights of both RTA and RTB are ˜32 kD.

Complete removal of the linker requires two distinct proteolytic events, and the nature of these cleavage events and the protease(s) responsible for them are not known. Studies by Sehnke, et al. have shown that tobacco can properly process native ricin toxin when transformed with the preproricin gene, suggesting that the proteolytic machinery necessary to remove the linker is present in plant species other than castor bean.⁵ Is it possible that this proteolytic machinery is responsible for the described breakdown pattern of RTB-containing fusion proteins? Do fusions at the N-terminus of RTB mimic the 12 amino acid linker sequence, and therefore trigger this proteolysis? If so, this would suggest that RTB sequences serve as recognition for the protease. Thus, it is of interest to determine whether this cleavage event is precise, what constitutes the recognition site, and whether modifications of this region will result in more stable fusion products.

Here we report the characterization of the breakdown phenomenon described above, through the use of N-terminal sequencing via Edman degradation chemistry. We outline strategies for the reduction of the breakdown and results attained.

Methods

### Constructs

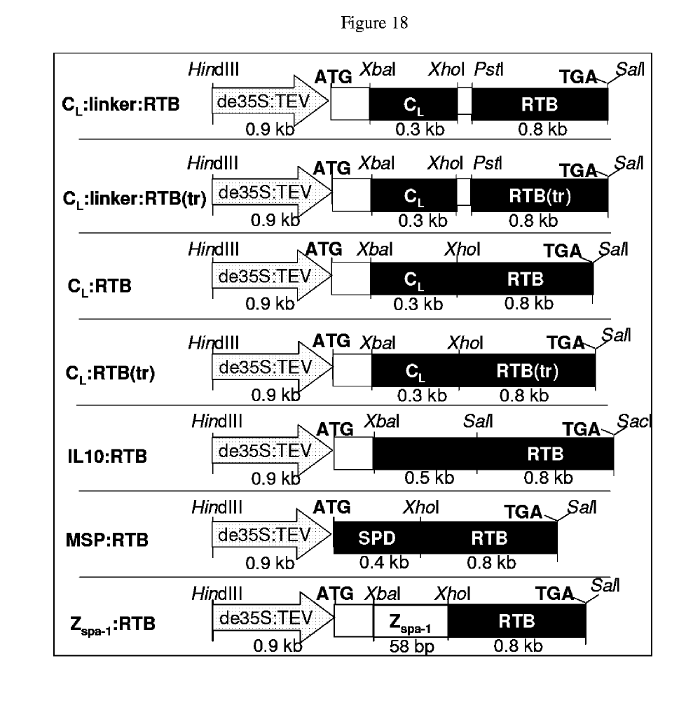

Maps of the constructs used in this research are shown in FIG. 18. Truncated RTB is denoted by “RTB(tr)”; IL-10 is the coding sequence of human interleukin-10; SPD refers to a propriety seed storage protein domain; Zₛₚₐ₋₁ is the 58 amino acid Protein A-binding affibody developed by Gräslund et al.; the light small boxes in the first and second constructs represent presence of the (Gly₃Ser)₃ flexible linker; the patatin signal peptide is denoted by “sp.” MSP:RTB uses a signal peptide endogenous to MSP. All were driven by the dual enhanced 35S CaMV promoter (de35S:TEV) for constitutive expression by the Agro-mediated transient system. All constructs were first created in the pBC (Stratagene) cloning vector or in a modified pBC in which the XhoI site of the polylinker had been removed (pBC-X; see Example 2 for details). Promoter:gene cassettes were subcloned into the pBIB-Kan binary vector⁶ for Agrobacterium tumefaciens-mediated transient expression in N. benthamiana via HindIII/SalI or HindIII/SacI. The map of the rRTB construct, used as a control in some studies here, can be found in Example 2, FIG. 6 (“Construct C”). The IL10:RTB construct was a gift of Dr. Maureen Dolan (Arkansas Biosciences Institute). The SPD:RTB construct was created by Jorge Ayala (Arkansas Biosciences Institute; Ayala, Dolan, Cramer, unpublished).

### Plant Growth Conditions, Transient Expression, and Recovery of RTB Fusions

N. benthamiana plants were grown and infiltrated with Agrobacterium tumefaciens as described in Example 1. Both initial enrichment and two step purification procedure for RTB are as described in Example 1.

### Point Mutations

Constructs creating point mutations within the RTB N-terminus were performed by Jorge Ayala (Arkansas Biosciences Institute), using the QuikChange II Site-directed mutagenesis kit (Stratagene, Cedar Creek Tex.), as per the manufacture's instructions. The template used in the reactions was the C_{L}:RTB construct in pBC. The primers used in the mutagenesis reaction were: 1) for RTBgdv 5′-GAGTGTCTCGAGGGTGATGTTTCTA (Forward) (SEQ ID NO: 35) and 5′-CCATAGAAACATCACCTCGAACACTC (Reverse) (SEQ ID NO: 36); 2) for RTBagv 5′-GAGTGTCTCGAGGCTGGTGTTTCTATGG (F) (SEQ ID NO: 37) and 5′-CCATAGAAACACCAGCCTCGAGACACTC (R) (SEQ ID NO: 38); 3) for RTBadg 5′-CTCGAGGCTGATGGTTCTATGGATCC (F) (SEQ ID NO: 39) and 5′-GGATCCATAGAACCATCAGCCTCGAG (R) (SEQ ID NO: 40).

### N-Terminal Sequencing

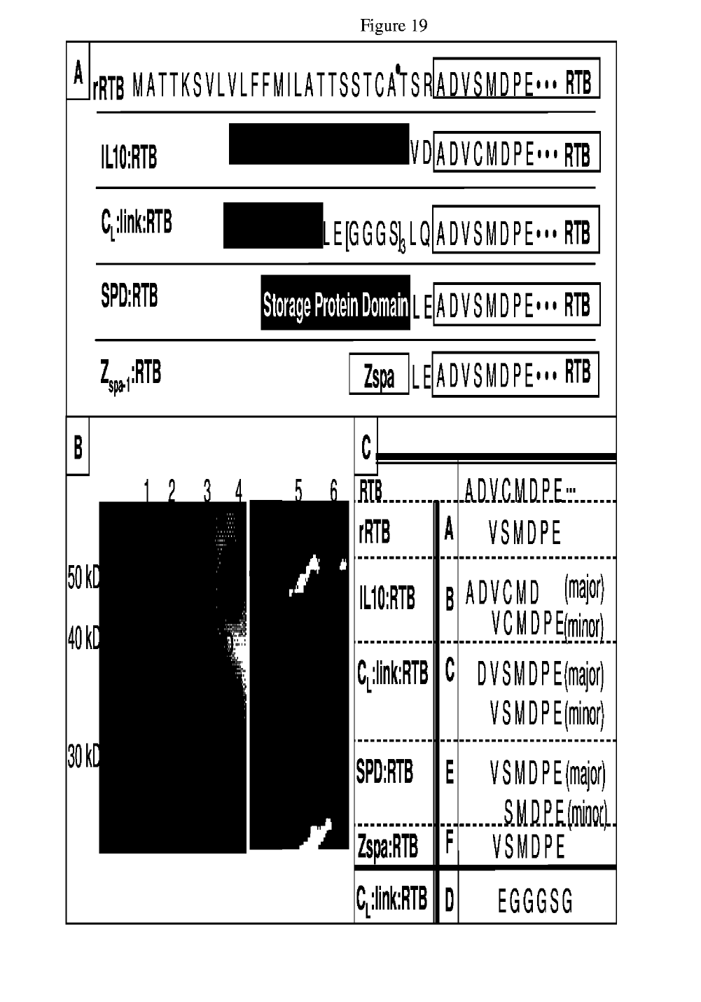

For preparation of samples, RTB-specific purified protein samples containing breakdown products to be sequenced were separated on 12% SDS-PAGE under reducing conditions and transferred to Sequi-Blot PVDF Membrane (Bio-Rad, Hercules Calif.). Following transfer, the membrane was stained with Coomassie and air dried. Bands to be sequenced were excised from the membrane and sent to the University of Virginia Health System Biomolecular Research Facility, Protein Sciences Division for N-terminal sequencing via Edman degradation chemistry. Alkylation of cysteine residues was not performed in our samples, resulting in identification of cysteine as serine (see FIG. 19C). FIG. 19C shows results of N-terminal sequences on indicated bands. The expected RTB N-terminal sequences is on top. Sequences of bands A, B, C, E and F are aligned with the expected sequences. Band D sequence derives from the (Gly₃Ser)₃ linker.

Results

### Characterization of Breakdown Phenomenon

Identification, removal, and/or exploitation of a proteolytic recognition site in RTB that results in a specific pattern of cleavage of N-terminal fusion partners strengthens the utility and flexibility of a RTB-based platform for delivery of proteins. Studying this phenomenon benefited from the fact that the major breakdown product retained full lectin activity. In fact, our hypotheses that this breakdown results from the machinery responsible for proper processing of ricin toxin rests on the notion that the breakdown product represents essentially the full length RTB protein (32 kD). Enrichment and purification schemes developed in other arenas of this dissertation research provided a readily available substrate for N-terminal sequencing. N-terminal sequencing of the 32 kD breakdown protein (32BP) allowed for direct inspection of the site at which any proteolysis may have occurred. Comparing 32BPs recovered from different constructs allowed for simple characterization of the phenomenon. Investigation of a breakdown pattern in which RTB was on the N-terminus of the fusion would necessitate a different purification scheme optimization for each construct.

Before any strategies to eliminate this degradation could be designed, it was first necessary to determine the N-terminal sequence of various 32BP species derived from different constructs. We chose C_{L}:(Gly₃Ser)₃:RTB (C_{L}:link:RTB), IL10:RTB, SPD:RTB, and Zₛₚₐ₋₁:RTB to investigate first because the fusion partners are quite different in terms of primary structure. The IL10:RTB construct is a fusion between human interleukin 10 and RTB, a gift of Dr. Maureen Dolan (Arkansas Biosciences Institute). SPD:RTB is a fusion of RTB and a proprietary seed storage protein domain. Zₛₚₐ₋₁:RTB is a fusion between RTB and the 58 amino acid affibody that binds to Protein A, and is used in some systems as a one-step purification tag.⁷ As a control, rRTB (see Example 1) was included. A representation of the fusion proteins selected can be in FIG. 19A, with the sequences of the patatin signal and junctions of the various fusions shown as well. (The sequences in 19A represented as SEQ ID NO: 42, 43, 44, 45, and 46 from top to bottom). The patatin signal peptide sequences is shown in “rRTB”. The dot above the sequence indicates the predicted signal peptidase cleavage site. The sequences of the junctions between fusion partners is shown, along with the first eight amino acids of RTB. The constructs were expressed in the Agrobacterium-mediated transient expression system and purified using the RTB-specific purification protocol described in Examples 1 and 2. Empty-vector (pBK) Agrobacterium-infiltrated leaves were used as purification control. Purified proteins were separated via reducing 12% SDS-PAGE, transferred to PVDF membrane and stained with Coomassie (see FIG. 19B). Bands A through F were N-terminally sequences. Lane 1 is empty vector (pBK)-infiltrated leaves run through purification scheme; lane 2, purified rRTB; lane 3, purified IL10:RTB and breakdown; lane 4, purified C_{L}:(Gly₃Ser)₃:RTB and breakdown banks; lane 5, SPD:RTB; lane 5, Zₛₚₐ₋ₐ:RTB. Analysis of RTB-purified proteins derived from plants expressing C_{L}:link:RTB (see FIG. 19, lane 4) revealed three bands that reacted with anti-RTB antibodies, the ˜45 kD band representing full length fusion protein and two bands in the ˜30-35 kD range. The smaller of these two bands was much higher in abundance relative to the heavier band. For IL10:RTB, two lactose-binding, anti-RTB reactive bands (data not shown) were observed for IL10:RTB, the larger intact fusion product (˜50 kD) and the 32BP. Purified proteins from both SPD:RTB and Zₛₚₐ₋₁:RTB infiltrated plants revealed only 32BP, and no visible full-length product. N-terminal sequencing revealed bands A (rRTB), B (32BP from IL10:RTB), and C (the smaller of the two breakdown bands from C_{L}:link:RTB), E (SPD:RTB), and F (Zₛₚₐ₋₁:RTB) all contained the sequence VSMDPE. Interestingly, rRTB, that is RTB that is not fused to another protein, does not match the expected sequence. The expected N-terminus of rRTB, considering the theoretical cleavage site of the patatin signal peptide⁸, is TSRADVSMDPE . . . (SEQ ID NO: 41) (see FIG. 19A), where the threonine derives from the signal peptide, and the serine and arginine are derived the XbaI restriction site used to clone the gene. The sequence of band D, EGGGSG, was unexpected. This protein was termed link:RTB, as it consists of the (Gly₃Ser)₃ linker on the N-terminus of RTB. The glutamine residue on the N-terminus derives from the PstI site used in the cloning of this fusion. Thus, it appears that there are two breakage events occurring in C_{L}:link:RTB, one at the N-terminus of the (Gly₃Ser)₃ linker (band D) and another at the N-terminus of RTB (band C).

### Generation of “Truncated” RTB

The three-dimensional structure of ricin and RTB reveal that the first six amino acids of RTB form a flexible “arm” that provides proximity of RTB Cys 4 and RTA Cys 259 to form disulfide linkage. This motif is found in other type II RIPs such as mistletoe lectin I and ebulin, even though amino acid identity is not well conserved in this region. The B-chains compared were of four type II RIPS, ricin B, (ADVCMDPEPI—SEQ ID NO: 47), Ebulin B (GETCAIPAPF—SEQ ID NO: 48), Misletoe lectin I B (CSASEPTVRI—SEQ ID NO: 49) and Abrin B (IVEKSKICSS—SEQ ID NO: 50). In most of our studies, Cys 4 of RTB has been mutated to Ser (see FIG. 19A), because of observed dimerization in other experiments. The inventors have discovered that by substituting the first cysteine residue of the ricin B chain subunit with another amino acid, it is possible to eliminate the site for sulfhydryl bonding which can cause bonding to other ricin B molecules or other proteins.

This change does not impact lectin activity, and since this change is not present in the IL10:RTB construct, it did not appear to be a factor in the observed breakdown phenomenon. To ask the question of whether this flexible portion mediates breakdown, either by serving as recognition for protease(s), or by introducing energetically unstable situations, we developed and tested constructs that fused C_{L} and C_{L}:linker directly to Pro 7 of RTB, thus eliminating the putative AD↓VC . . . cleavage site. This so-called “truncated” RTB was termed RTB(tr). Pro 7 was chosen for two reasons. First, Pro 7 is the first amino acid that resides on the edge of the “core” of RTB based on the crystal structure of ricin (PDB ID#2aai).³ Second, many proteases, such as trypsin, will not cleave peptide bonds involving proline.

### Impact of Amino Acids 1 Through 6 of RTB on Breakdown Phenomenon

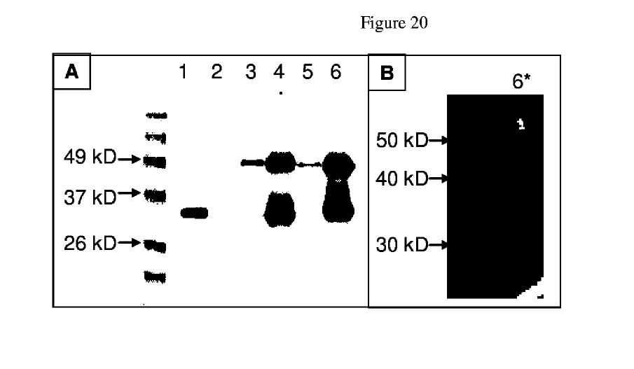

The first construct created incorporating RTB(tr) was C_{L}:linker:RTB(tr), where “linker” is (Gly₃Ser)₃ (see FIG. 18). C_{L}:linker:RTB and C_{L}:linker:RTB(tr) was expressed using the Agrobacterium-mediated transient system, and leaves were collected at 24 and 48 hr. Western analysis using anti-RTB antibodies of lactose-enriched proteins revealed that at the 48 hr time point, only one breakdown band was visible in C_{L}:linker:RTB(tr), where two were observed in C_{L}:linker:RTB, as previously demonstrated (see FIG. 20A). FIG. 20A shows Western blot analysis of C_{L}:link:RTB(tr), lane 1, 30 ng cbRTB; lane 2, empty vector (pBK)-infiltrated leaves; lane 3, 24 hour post-infiltration C_{L}:link: RTB; lane 4, 48 hour C_{L}:link:RTB; lane 5, 24 hour C_{L}:link:RTB(tr); lane 6, 48 hours C_{L}:link:RTB(tr); and lanes 2 through 6 are lactose-binding fractions of infiltrated leaf extracts. Comparisons of lanes 4 and 6 in FIG. 20A show that the breakdown band observed in C_{L}:linker:RTB(tr) migrates to a position on SDS-PAGE that is between the positions of the two breakdown bands seen in C_{L}:linker:RTB. A slightly smaller full-length C_{L}:linker:RTB(tr) compared to C_{L}:linker:RTB is also observed, consistent with the construct-mediated elimination of six amino acids. FIG. 20B shows a Coomassie-stained membrane of purified C_{L}:linker:RTB(tr) (from the 48 hr time point), and it also shows only one breakdown band (compare to FIG. 19B lane 4). Sequencing of Band G in FIG. 20B (the breakdown product of C_{L}:linker:RTB(tr)) reveal the N-terminal sequence to be EGGGSG, the same sequence seen in Band D (the higher molecular weight breakdown band from C_{L}:linker:RTB, see FIG. 19B). Band G is termed linker:RTB(tr). This is consistent with our previous result that the (Gly₃Ser)₃ linker is susceptible to cleavage in N. benthamiana. However, lack of other N-terminal sequences within Band G reflects that the removal of the A D V S/C M D sequence stabilizes the fusion. In order to confirm this without the “complication” of the linker-specific breakdown susceptibility, we created additional constructs lacking this linker (see FIG. 18).

Generation of Constructs without (Gly₃Ser)₃ Linker

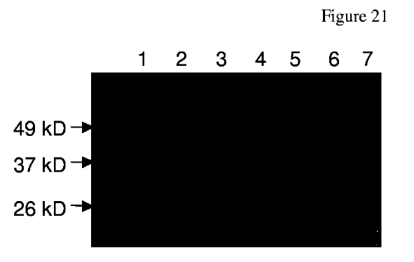

In order to confirm the role of this six amino acid sequence, A D V C/S M D, in mediating degradation of N-terminal fusions to RTB, constructs which omit the (Gly₃Ser)₃ linker sequence were created, termed C_{L}:RTB and C_{L}:RTB(tr) (see FIG. 18). Anti-RTB western analysis comparing the lactose-binding fractions generated from the infiltration of five different constructs: an empty-vector (pBK) control, rRTB, C_{L}:link:RTB, C_{L}:link:RTB(tr), and C_{L}:RTB(tr) is shown in FIG. 21. (Lane 1: 30 ng cbRTB; lane 2, pBK; lane 3, rRTB; lane 4, C_{L}:link:RTB; lane 5, C_{L}:link:RTB(tr); lanes 6 and 7, CL:RTB(tr).) The expression of C_{L}:RTB(tr) is very low compared to the others, and a ˜45 kD band is only visible after a long (45 min) exposure time. The fact that there is only one band present (lane 6) indicates that fusing C_{L} to Pro 7 prevents degradation.

Point Mutations within the First Three Amino Acids of RTB

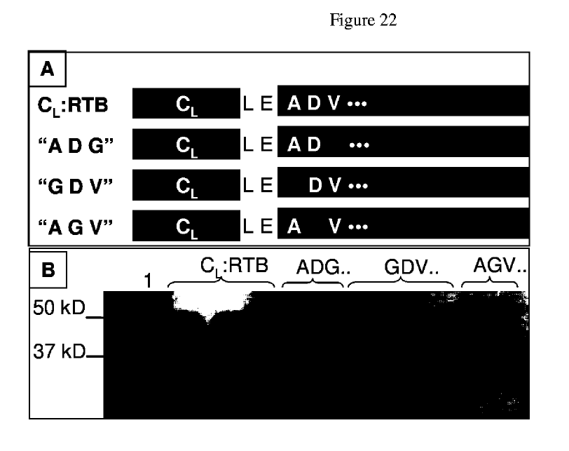

Results described above indicate that the first six amino acids of RTB constitute a potential proteolytic sensitive site. As a first step in further delineating this site, a series of constructs were generated that created single amino acid replacements within this region. Point mutations in which the first, second, or third amino acid of RTB in the C_{L}:RTB construct was changed to glycine were created to determine if any of these individual amino acids are critical to the observed degradation. The specific changes are delineated in FIG. 22A. The mutation occurs at “G” in ADG, GDV and AGV. “ADG is Val 3 Δ Gly, “GDV is Ala 1 Δ Gly. Anti-RTB Western analysis of lactose-binding fractions generated from these constructs can be seen in FIG. 22B, with lane 1 30 ng cbRTB. All three constructs produced identical patterns of degradation, indicating that the first three amino acids of RTB are not individually responsible for the observed breakdown.

### Discussion

We have reported the preliminary characterization of a phenomenon in which proteins fused to the N-terminus of RTB are cleaved off. It has been shown that the first six amino acids of RTB, A D V C/S M D, facilitate this breakdown.

### REFERENCES

1. Lamb, F. I., Roberts, L. M., & Lord, J. M. Nucleotide sequence of cloned cDNA coding for preproricin. Eur. J. Biochem. 148, 265-270 (1985).
2. Frigerio, L., Jolliffe, N. A., Di Cola, A., Felipe, D. H., Paris, N., Neuhaus, J., Lord, J. M., Ceriotti, A., & Roberts, L. M. The internal propeptide of the ricin precursor carries a sequence-specific determinant for vacuolar sorting. Plant Physiology 126, 167-175 (2001).
3. Montfort, W., Villafranca, J. E., Monzingo, A. F., Ernst, S. R., Katzin, B., Rutenber, E., Xuong, N. H., Hamlin, R., & Robertus, J. D. The three-dimensional structure of ricin at 2.8 A. J. Biol. Chem. 262, 5398-5403 (1987).
4. Rutenber, E. & Robertus, J. D. Structure of Ricin B-Chain at 2.5 A Resolution. Proteins 10, 260-269 (1991).
5. Sehnke, P. C. & Ferl, R. J. Processing of preproricin in transgenic tobacco. Protein Expression and Purification 15, 188-195 (1999).
6. Becker, D. Binary vectors which allow the exchange of plant selectable markers and reporter genes. Nucl. Acid. Res. 18, 203-210 (1990).
7. Gräslund, S., Eklund, M., Falk, R., Uhlén, M., Nygren, P.-Å., & Ståhl, S. A novel affinity gene fusion system allowing protein A-based recovery of non-immunoglobulin gene products. J. Biotechnol. 99, 41-50 (2002).
8. Iturriaga, G., Jefferson, R. A., & Bevan, M. W. Endoplasmic reticulum targeting and glycosylation of hybrid proteins in transgenic tobacco. The Plant Cell 1, 381-390 (1989).

### Example 4

Protocols and Results for Production and Testing of RTB-ER Rationale and Objectives. RTB has significant utility in facilitating the delivery of associated payloads across mucosal surfaces and into the endomembrane system on a diverse array of animal cell types. However, the majority (estimates of 80-90%) of RTB and associated payload accumulates in endosomal/lysosomal compartments. A small amount is directed to the endoplasmic reticulum (ER) via the retrograde pathway based on RTB binding to calreticulin. There are many therapeutic applications for which efficient delivery of chemicals, proteins or polynucleotide (RNA, DNA) to the ER would be beneficial. We reasoned that addition of the ER retrieval signal (KDEL or HDEL) to the amino-terminus of RTB would result in directing a significant proportion into the ER rather than into lysosomes. To test this hypothesis, we developed an RTB-KDEL construct, introduced it into an expression vector, expressed it in leaves of Nicotiana benthamiana via Agrobacterium-mediated transient expression, purified and characterized the resulting “RTB-ER” product, and assayed it for uptake and subcellular localization in mammalian cells.

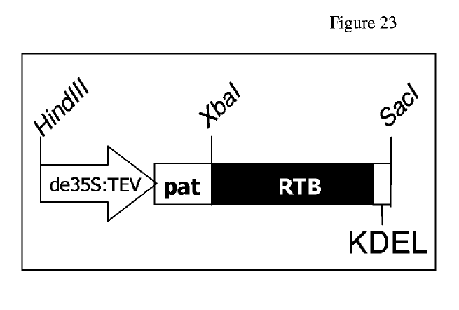

Plasmid Construction. Sequences encoding RTB were amplified by PCR using Vent_{R} DNA polymerase (New England BioLabs, Beverly, Mass.), DNA template R6-2 (Medina-Bolivar, et., al 2003) and the oligonucleotides 5′-GCACTCGAGAAATAATGGTAACC-3′ (SEQ ID NO: 51) and 5′-CGCTCTAGAGCTGATGTTTGTATGGAT-3′ (SEQ ID NO: 52) which introduced an XbaI and XhoI site at the 5′ and 3′ end of RTB, respectively. Complementary KDEL oligonucleotides 5′-GATCCTCGAGAAGGATGAGCTTTGAGAGCTCGATC-3′ (SEQ ID NO: 53) and 5′-GATCGAGCTCTCAAAGCTCATCCTTCTCGAGGATC-3′ (SEQ ID NO: 54) carrying an XhoI site at the 5′ end and the stop codon TGA and Sad sites at the 3′ end were mixed in equal amount and annealed by heating-cooling procedure before digestion. An XbaI/SacI R6-2 plasmid carrying the dual enhanced cauliflower mosaic virus (CaMV) 35S promoter, the tobacco etch virus enhancer (TEV) translational enhancer, and the patatin signal peptide from potato (de35S:TEV::Pat) was used as vector template for subcloning of the properly digested RTB and KDEL fragments (See FIG. 23). Correct ligation was confirmed by sequencing. The 1.8 kb HindIII/SacI cassette was then moved to the binary vector pBIB-Kan (Becker, 1990) and the resulting plasmid was introduced into Agrobacterium tumefaciens strain LBA4404 by freeze/thaw method (Holsters et., al 1978).
Transient expression in Nicotiana benthamiana. Cultures of A. tumefaciens harboring the pBIB-Kan promoter:RTB-KDEL gene cassette were grown in 50 mL YEP medium (10 g/L bacto-peptone, 10 g/L yeast extract, 5 g/L NaCl) containing 100 μg/mL kanamycin and 60 μg/mL streptomycin for 48 hr at 28° C., 220 rpm. To induce A. tumefaciens prior to infiltration, cell pellets were harvested via centrifugation (5000×g for 10 min), resuspended in 300 mL induction media (20 mM MES pH 5.5, 0.3 g/L MgSO₄.7H₂O, 0.15 g/L KCl, 0.01 g/L CaCl₂, 0.0025 g/L FeSO₄.7H₂O, 2 mL/L 1 M NaH₂PO₄ pH 7.0, 10 g/L glucose) containing 100 μg/mL kanamycin and 60 μg/mL streptomycin, supplemented with 0.2 μM acetosyringone and incubated at 28° C., 220 rpm, for 4 hr to overnight. Induced A. tumefaciens cultures were introduced into four to six week old Nicotiana benthamiana plants by vacuum infiltration. For vacuum infiltration, plants were place upside-down in a beaker containing the induced culture so that all aerial portions were submerged. The beaker and plant was then placed inside a vacuum chamber and vacuum was applied (approximately 1 min) and broken by abruptly pulling off the tube from the chamber. This procedure was performed twice for each plant to ensure complete infiltration. Following infiltration, plants were replaced to environmental growth chambers. Leave were harvested 72 h pos-infiltration and stored at −80 if not used immediately.
Quantification of RTB:KDEL in crude extract. Infiltrated leaves, 0.5 g were ground to a fine powder in liquid nitrogen and then 1.5 ml of extraction buffer (75 mM NaH₂PO₄, 25 mM Na₂HPO₄, 150 mM NaCl pH 7.4) was added. After thawing at RT, the sample was transferred to a 2.0 ml microcentrifuge tube and centrifuged at 13000 rpm/4 C/30 min. The supernatant was then transferred to a clean microcentrifuge tube and aliquots were then quantified for RTB levels utilizing an asialofetuin assay.
Asialofetuin assay. A functional ELISA utilizing asialofetuin instead of a capture antibody was employed to assess galactose-specific lectin activity and quantify RTB:KDEL fusion proteins. Asialofetuin is a modified mammalian glycoprotein that contains galactose-terminated glycans (Sigma, St. Louis Mo.) and thus serves as a strong binding target for functional RYB. Asialofetuin at 300 μg/mL in PBS was bound to the wells of an Immulon 4HBX plate for 1 hr at RT. The wells were then blocked with 3% BSA in PBS for 1 hr at RT. Castor bean-derived RTB (cbRTB; Vector Labs, Burlingame Calif.) was used for the standard curve, ranging from 1.95 to 250 ng/well in PBS+10 mM D-galactose. For asialofetuin binding, 100 μL of standards and samples incubated at RT for 1 hr. The plate was then washed 3× with PBS (300 μL/well). Rabbit anti-Ricinus communis lectin antibody (Sigma R-1254), diluted to 1:4000 in blocking buffer was then added (200 μL/well) and allowed to incubate for 1 hr at RT. Wells were washed again and alkaline phosphatase-labeled goat anti-rabbit antibody (Bio-Rad, Hercules Calif.), diluted 1:4000 in blocking buffer, was added and allowed to incubate for 45 min at RT. The wells were washed a third time and alkaline phosphatase substrate (100 μL/well; Pierce, Rockford Ill.) was applied. After the color developed sufficiently (10-15 min), the reaction was stopped with the addition of 50 μL 2 N NaOH and the absorbance at 405 nm was read. The inverse of the absorbance at 405 nm was plotted vs. the inverse of the standard RTB/well to give a linear relationship. This equation was then used to estimate the amount of RTB in the crude extract and FPLC purified samples.
RTB:KDEL purification by FPLC. Agro-infiltrated leaves, 10-20 g of infiltrated leaves were ground under liquid N₂ in a mortar and pestle to a fine powder. 50 mL of extraction buffer 2 (100 mM Tris-HCl pH 7.5, 20 mM D-galactose, 1% PVPP) was added to the powder and allowed to thaw at RT. The resulting crude extract was centrifuged at 14,200×g for 30 min at 4° C. The supernatant was filtered through KimWipes, brought to 100 mL with distilled H₂O, and the pH was adjusted to 7.5 with 1 N NaOH. This cleared extract was then filtered though a 0.45 μm membrane and loaded onto an equilibrated 20 mL column volume MacroPrep High Q (Bio-Rad, Hercules Calif.) column using a Bio-Rad Duo-Flow FPLC system. Following loading of the sample, the column was washed with 80 mL of 50 mM Tris-HCl pH 7.5. The RTB-containing proteins were eluted and collected from the column by washing with 45 mL 50 mM Tris-HCl pH 7.5, 400 mM NaCl. The column was then cleaned by washing with 50 mM Tris-HCl pH 7.5, 1 M NaCl and re-equilibrated with 50 mM Tris-HCl pH 7.5. The RTB-containing sample (400 mM NaCl) was loaded onto a 1 mL immobilized lactose column (EY Laboratories, San Mateo Calif.) and washed with PBS. Purified RTB-KDEL proteins were eluted by washing with 4×1 mL PBS+500 mM D-galactose. RTB-containing samples were then concentrated using YM-10 Centricons (Millipore Corp., Bedford Mass.) and dialyzed to PBS. Concentrated, dialyzed samples were then analyzed via silver stained SDS-PAGE and asialofetuin binding assay. SDS-PAGE was performed using 10% or 12% PAGE-gels (PAGE-gel, Inc. San Diego, Calif.). Silver staining was performed using the SilverSnap kit (Pierce, Rockford, Ill.).
Results. Quantification of RTB:KDEL in the crude extract yielded 27.43 μg of RTB/g FW. Using a buffer with 20 mM D-Galactose will probably increase the yield as we have observed in other experiments. Purified RTB-KDEL and RTB were fluorescently labeled with fluorescamine and compared for uptake into human epithelial cells (HT29 cells). Cells were counter-stained with ER-Tracker-Red, a fluor that specifically localized to the ER. RTB localized primarily to endosomal/lysosomal compartments as visualized by punctuate staining. The pattern of green fluorescence (RTB) did not overlap with the red (ER-Tracker-Red). In contrast, the pattern of RTB-KDEL fluorescence shows significant overlap with the ER-Tracker-RED, as visualized as a yellow/orange fluorescence in composite images that overlay red (ER-Tracker) and green (RTB-KDEL) fluoresce.
Conclusions. RTB engineered to contain a KDEL ER-retrieval signal, localizes primarily to ER compartments upon uptake into mammalian cells. We have termed this product “RTB-ER”.

### REFERENCES

1. Becker, D. Binary vectors which allow the exchange of plant selectable markers and reporter genes. Nucl. Acid. Res. 18, 203-210 (1990).
2. Holsters, M., de Waele, D., Depicker, A., Messens, E., van Montagu, M., & Schell, J. Transfection and transformation of Agrobacterium tumefaciens. Mol. Gen. Genet. 163, 181-187 (1978).
3. Medina-Bolivar, F., Wright, R., Funk, V., Sentz, D., Barroso, L., Wilkins, T. D., Petri, W., & Cramer, C. L. A non-toxic lectin for antigen delivery of plant-based mucosal vaccines. Vaccine 21, 997-1005 (2003).

### Example 5

### Introduction

The immuno-modulating cytokine, interleukin-12 (IL-12), has great potential as an anti-tumor therapeutic and vaccine adjuvant for cancers and viral infections. However, its clinical application was hindered by severe side-effects associated with systemic administration. Several studies have suggested that mucosal delivery of IL-12 is as effective as systemic administration for immune modulation with much reduced toxicity. Ricin B (RTB), the non-toxic carbohydrate-binding subunit of ricin, is essential to the uptake of ricin into mammalian cells and the intracellular trafficking of ricin A, the catalytic subunit of ricin, from the endomembrane system to the cytosol, where ricin A inactivates ribosomes. RTB's function suggests that it may work as a molecular carrier for effective mucosal delivery of IL-12. To test this hypothesis, transgenic plants producing RTB:IL-12 fusions were generated and characterized. Our results demonstrated that RTB fused to the carbonyl-terminus of IL-12 maintained full lectin activity and IL-12 bioactivity. RTB fused to the amino-terminus of IL-12 did not show lectin activity due to steric hinderance. Purified IL-12:RTB from transgenic plant tissue was tested in an in vitro mucosal-associated lymphoid tissue (MALT) assay. The results indicate that RTB facilitates the binding of IL-12 to the epithelial cells and presentation of IL-12 to immune responsive cells.

### Introduction

IL-12 is a very important immuno-modulator in that it enhances cell-mediated immunity (CMI) and inflammation. It stimulates the secretion of interferon-gamma (IFN-γ) from T cells and natural killer (NK) cells and activates the innate resistance to infections. It also plays vital roles in the maturation and differentiation of type 1 T helper cells (Th1) and cytotoxic T lymphocytes (CTL) (Trinchieri, 1994; 2003). It shows great potential as an anti-tumor therapeutic and adjuvant for cancer and viral vaccines (reviewed by Colombo and Trinchieri, 2002). However, its clinical application was hindered by severe side-effects associated with systemic administration (Leonard et al., 1997). Localized presentation through subcutaneous gels or intranasal administration has suggested that mucosal delivery may allow for the desired IL-12 efficacy at much lower concentrations and thus greatly reduced toxicity (Huber et al., 2003; Salem et al., 2004).

Ricin, a type II ribosome-inactivating protein (RIP) plant toxin from castor bean (Ricinus communis), is a heterodimer consisting of two subunits, ricin A (RTA) and ricin B (RTB). RTA, the catalytic subunit, has N-glycosidase activity and inactivates the ribosomes (Endo et al., 1987). RTB, the galactose/galactosamine-binding subunit of ricin, binds to glycan-rich mammalian cell surfaces so that ricin is internalized into cells through receptor-mediated endocytosis (Mohanraj et al., 1995). Many investigations suggest multiple subcellular transport pathways for ricin following endocytosis (reviewed by Sandvig et al., 2000). Indirect evidence suggests that ricin reaches the endoplasmic reticulum (ER) where RTB is degraded and RTA is translocated to the cytosol by translocons (ER-localized Sec61; Roberts and Smith, 2004). Ricin may also be transported from endosomes to lysosomes and becomes digested (Magnusson et al., 1993), or recycled to the cell surface by transcytosis (van Deurs et al., 1990).

RTB plays important roles in these multiple transport pathways of ricin. The two galactose-binding domains of RTB are essential to the cytotoxicity of ricin (Newton et al., 1992), because it not only facilitate the internalization of ricin into the cells but also aids in the retrograde transport of RTA from the endosomes to the ER (reviewed by Roberts and Smith, 2004). In addition, RTB is a glycoprotein. The mannosylated glycans also interact with the D-mannose receptor on the surface of certain type of cells (such as macrophages) and ricin enters cells via the clathrin-coated pit pathway (Frankel et al., 1997).

RTB's involvement in multiple transport pathways into and within the cell is utilized here as a molecular carrier to facilitate the localized presentation of antigens or cytokines, such as IL-12, to the mucosal immune responsive cells. Disarmed ricin fused with a small peptide facilitated the presentation of the small peptide to MHC class I molecules, indicating disarmed ricin could be an adjuvant for cancer vaccines (Smith et al., 2002). Medina-Bolivar et al. (2003) found that transgenic tobacco-produced RTB worked as mucosal carrier/adjuvant and mediated the induction of primarily Th2-skewed immune responses to its fusion partner GFP.

Previous research has already shown that transgenic plants are able to produce bioactive RTB lectin and IL-12 cytokine, alone (Medinar-Bolivar et al., 2003; Reed et al., 2005; Kwon et al, 2003; Gutierrez-Ortega et al, 2004; 2005) but have not shown a fusion protein of IL-12:RTB. In order to test the potential of RTB to function as a mucosal carrier and fusion partner for IL-12, transgenic plants were generated to produce RTB fused to the single-chain form of murine IL-12 (mIL-12). The lectin activity of mIL-12:RTB fusions were determined by their ability to bind to asialofetuin, a galactose-rich protein, in a microtitre plate-binding assay (Dawson et al., 1999). Purified mIL-12:RTB fusion products showed both IL-12 biological activity and lectin activity. An in vitro mammalian cell culture model was utilized to demonstrate that RTB acts as a molecular carrier and may facilitate the uptake of mIL-12 into mucosal associated lymphoid tissue (MALT).

Hairy roots were produced following Agrobacterium transformation based on the following general protocols. The constructs were mobilized into the Agrobacterium tumefaciens strain LBA4404 by a modified freeze-thaw method (Chen et al., 1994). Transformation of Nicotiana tabacum cv. Xanthi was performed using a petiole transformation procedure (Medina-Bolivar and Cramer, 2004). Following a 48 hour incubation of Agrobacterium-infected leaf explants on Murashige and Skoog Basal Salt media (MS; GIBCO), leaves were transferred to supplemented MS media (0.1 mg/l 1-naphthalene acetic acid, 1 mg/l 6-benzylamine purine, 500 mg/l carbenicillin and 250 mg/l kanamycin) to facilitate transgene integration into plant cells and to provide selection of regenerated transgenic shoots. Antibiotic-selected plantlets were screened for the presence of transgene by PCR using primers. Transgenic plants generated with highest expression levels (see ELISA screening below) were maintained for sterile propagation as fully rooted plants in sterile agar media. Select lines were also transferred to soil and taken to seed. Analogous cultures of non-transgenic tobacco plants were maintained and used as controls for this study.

Hairy roots from select, high-expressing transgenic plants and wild type plant were also established by a procedure previously described (Medina-Bolivar and Cramer, 2004). Briefly, Agrobacterium rhizogenes (ATCC 15834) was introduced at wound sites created by cutting longitudinally along the midrib of excised scmIL-12 expressing transgenic leaves. Infected leaves were incubated on solid MS media for two weeks to allow hairy root development at wound site. Individual root tips representing independent hairy root clonal lines were excised and transferred to B5 medium containing cefotaxime to remove Agrobacterium rhizogenes. Liquid cultures were initiated with ˜20 root tips (1 cm) in a 250 ml flask containing B5 media (50 ml) and maintained under continuous light with shaking (90 rpm). For routine, scale-up production of mIL-12, 1-week old hairy root cultures (50 ml media/250 ml flask) were transferred to PYREX® 2800 mL Fernbach-Style Culture Flask (Item #4420-2XL, Corning) containing 0.5 L media and cultured for an additional 2 weeks. Typically each flask yielded 20-30 g of root biomass; tissue was stored at −80° C. upon harvest.

In the ELISA assay, a heterodimer-specific mIL-12 enzyme-linked immunoabsorbent assay (ELISA) was used for the detection and quantitation of plant-derived mIL-12 (R&D Systems). This assay will be referred to as the “conformational” mIL-12p70 ELISA for it exclusively detects only correctly folded and/or assembled mIL-12p70 protein. Plant tissue was ground in 2 volumes of extraction buffer (100 mM Tris base, 100 mM ascorbic acid, 150 mM NaCl, 4 mM EDTA, 2.5% PVP-40 0.1% Tween 20, pH7.0) and cell-free supernatants were analyzed on Immunlon 4 HBX plates (Thermo Labsystems) coated with 1 μg/ml rat anti-mIL12p70 monoclonal capture antibody (clone 48110.111; R&D Systems). Following a block step in PBST (0.1% of Tween 20 in PBS, 137 mM NaCl, 2.7 mM KCl, 10 mM Na₂HPO₄, 2 mM KH₂PO₄) with 1% BSA, serial dilutions of standard recombinant mIL-12 (R&D Systems), plant-derived mIL-12 samples and equivalent extracts from non-transgenic roots were loaded. Captured mIL-12p70 was detected with 100 ng/ml biotinylated anti-mIL-12 antibody (R&D Systems), streptavidin HRP (R&D Systems), and KPL substrate solution. Plates were read at 450 nm. All quantitation, purification screens and specific activity calculations for plant-derived mIL-12 were based on this conformational p70 ELISA using the commercially-available insect cell-derived mIL-12 as standard (R&D Systems).

The proliferation response of mouse splenocytes to plant-derived mIL-12 was measured for equivalent bioactivity to the animal cell-derived mIL-12 standard (R&D Systems). Briefly, mouse splenocytes were isolated and plated at a concentration of 7.5×10⁵/ml in media (RPMI 1640, 5 mM HEPES, 2 mM glutamine, 10% heat inactivated FBS, penicillin/streptomycin, 50 μM 2-ME, 10 ng/ml rhIL-2) and cultured for 3 days in the presence of the mitogen, PHA (10 μg/ml; Sigma). Splenocytes were collected, washed and resuspended in assay media (RPMI 1640, 20 ng/ml PMA, 5 mM HEPES, 2 mM glutamine, 10% heat inactivated FBS, penicillin/streptomycin and 50 μM 2-ME) and plated at 100 μl of 4×10⁵ cells/ml with various concentrations of animal cell-derived mIL-12 (R&D Systems) or equivalent amounts of plant-derived samples (quantitation based on mIL-12 ELISA detailed above). Following a 2-day culture period, cell proliferation rates were compared in a standard colorimetric assay analyzed at O.D. 490 nm (Promega Substrate CellTiter 96 Aqueous One Solution Reagent).

### Material and Methods

### Construction of RTB:IL-12 Gene Fusion

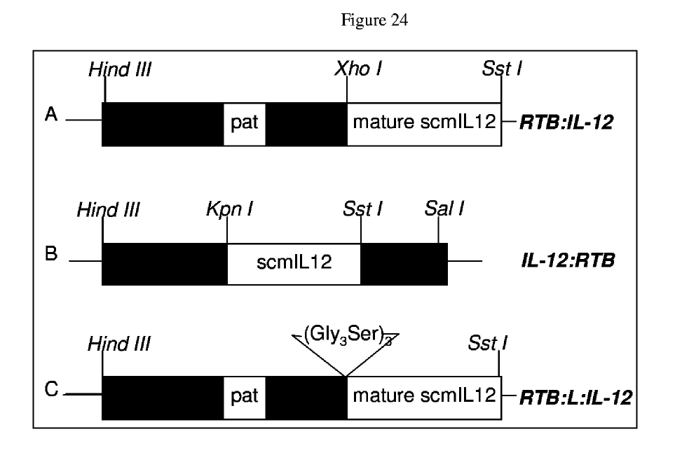

IL-12 sequences encoding the “mature” single chain murine IL-12 (scmIL-12, lacking the p40 signal peptide) were amplified from plasmid pSFG-mIL-12.p40.L.delta.p35 (Lieschke et al., 1997) by PCR using primer 5′-CTCGAGATGTGGGAGCTGGAGAAAG (SEQ ID NO: 55) and primer 5′-GAGCTCTCAGGCGGAACTCAGATAG (SEQ ID NO: 56) which incorporated flanking restriction enzyme sites XhoI and Sad, respectively. A DNA fragment containing the constitutive 35S promoter (double enhanced 35S promoter; Becker, 1990), the TEV translational enhancer (Carrington et al., 1990), and the sequences encoding the patatin signal peptide (pat, Iturriaga et al., 1989) and ricin B subunit 35S:pat:RTB was obtained by digesting plasmid R6-2 (Medina-Bolivar et al., 2003) with HindIII and XhoI. Plasmid pBC (Stratagene, La Jolla, Calif.) was digested with HindIII and Sad and ligated in a tri-molecular reaction with IL-12 and 35S:pat:RTB fragments to yield plasmid RTB:IL-12. See FIG. 24 where de35S:TEV refers to the double enhanced 35S promoter with TEV translational enhancer, mature scmIL-12:DNA fragment encoding murine IL-12 without endogenous signal peptide, scmIL-12:DNA fragment encoding murine IL-12 (p40-p35) with mIL-12p40 endogenous signal peptide and RTB:DNA fragment decoding ricing B subunit.

### Construction of RTB:L:IL-12 Plasmid

In order to place a flexible linker between the IL-12 and RTB compounds, a pair of oligonucleotides encoding (Gly₃Ser)₃ flanked with XhoI restriction sites on both ends (5′-ACGCTCGAGGGAGGTGGATCAGGTGGCGGATCTGGTGGAGGTTCTCTCGA GTAC) (SEQ ID NO: 57) was synthesized (MWG-Biotech, High Point, N.C.). After annealing, the double-stranded oligo was digested by XhoI.

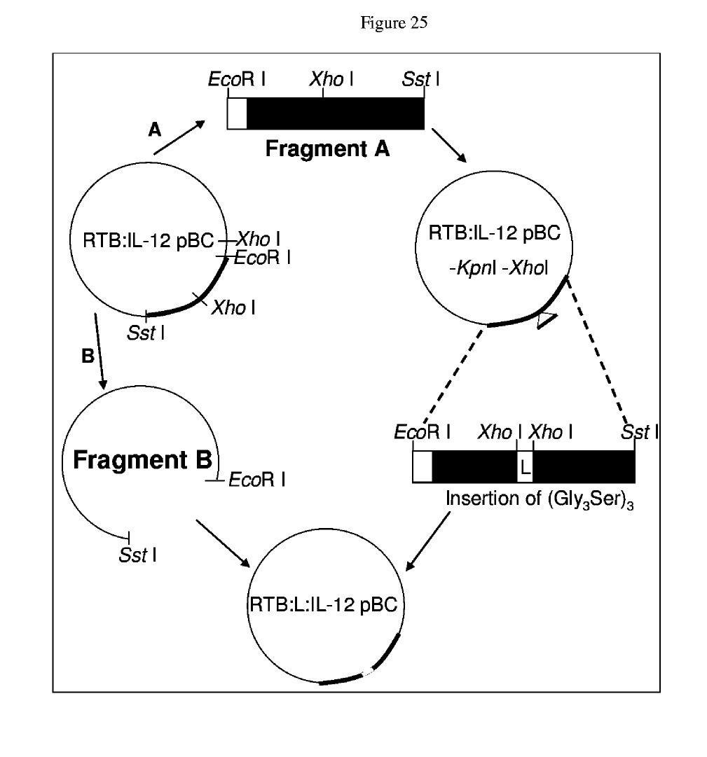

RTB:IL-12 construct in pBC (described above) was digested with EcoRI and Sad so that the construct was cleaved into two fragments, A and B (FIG. 25). Fragment A (EcoRI-RTB:IL-12-SstI) containing TEV:pat:RTB:IL-12 was inserted into a pBC vector in which KpnI and XhoI sites were eliminated by digestion with Mung Bean Nuclease (New England BioLabs, Boston, Mass.). The consequent construct was then digested by XhoI and ligated with the XhoI-digested linker fragment. Following sequencing confirmation, fragment EcoRI-RTB:linker:IL-12-Sac I was digested out and inserted back into fragment B. This construct was called RTB:L:IL-12 (FIG. 25).

### Construction of IL-12:RTB Gene Fusion

For construction of the IL-12:RTB plant expression vector, an IL-12 fragment (KpnI-p40:L:p35-SacI) without the stop codon was amplified from pSFG-mIL-12.p40.L.delta.p35 (Lieschke et al., 1997) by PCR with primer 5′-GGTACCATGGGTCCTCAGAAGCTAA (SEQ ID NO: 58) and primer 5′-GAGCTCGGCGGAACTCAGATAGCC (SEQ ID NO: 59). Sequences encoding RTB were amplified from plasmid R6-2 (Medina-Bolivar et al., 2003) using primers 5′-GAGCTCGCTGATGTTTGTATGGA (SEQ ID NO: 50) and 5′-GTCGACTCAAAATAATGGTAACCATA (SEQ ID NO: 9) which added a SacI site to the 5′ end and an in-frame stop codon and SalI site to the 3′ end. DNA fragments including the double enhanced 35S: TEV promoter digested from plasmid R6-2 (Medina-Bolivar et al., 2003), IL-12 and RTB with stop codon were assembled into pBC by multiple digestions and ligations to yield the construct IL-12:RTB (FIG. 24).

### Plant Transformation

All constructs were introduced into the binary vector pBIBKan (Becker, 1990) and transformed into tobacco (Nicotiana tabacum) cv. Xanthi using Agrobacterium-mediated transformation.

### IL-12 ELISA

The expression levels of IL-12:RTB and RTB:IL-12 were determined by IL-12 ELISA. It should be noted that this ELISA uses the p70 monoclonal antibody as the capture antibody. This antibody only binds to IL-12 in the proper conformational structure.

### Asialofetuin Binding Assay

Crude extracts from transgenic plants expressing the various RTB fusions were investigated for RTB carbohydrate-binding activity using an asialofetuin-binding assay (Dawson et al., 1999). Microtiter plates were coated with 300 μg/ml of asialofetuin (calf fetuin digested with sialidase, Sigma) in PBS (137 mM NaCl, 2.7 mM KCl, 10 mM Na₂HPO₄, 2 mM KH₂PO₄), 100 μl/well, overnight at room temperature. After being blocked with 1% BSA in PBS for 1 hour, 96-well plates were incubated with samples for 1 hour. For detection of bound RTB, plates were subsequently incubated with anti-ricin rabbit IgG (1:3000, Sigma, St. Louis, Mo.), goat anti-rabbit IgG conjugated with alkaline phosphatase (1:3000, Bio-Rad, Hercules, Calif.) and phosphatase substrate (Pierce) and absorbance was read at 415 nm.

### SDS-PAGE and Western Blotting Analysis

Plant tissue was ground in two volumes of PBS containing 20 mM galactose. Crude extracts (20 μg of total soluble protein per sample) or purified samples were boiled with 1×SDS gel loading buffer for 5 min and resolved by 10% SDS-PAGE (Invitrogen, Carlsbad, Calif.). Proteins were subsequently stained using a silver stain kit (GBiosciences, St. Louis, Mo.) or Coomassie blue, or blotted onto nitrocellulose membranes (Bio-Rad). For polyclonal anti-mIL-12 western blotting, membranes were subsequently blocked with 1% BSA in PBST (PBS with 0.1% of Tween 20) for 1 hour at room temperature, incubated with goat anti-mIL-12 neutralizing antibody (1:10,000, R&D Systems, Minneapolis, Minn.) in 1% BSA/PBST for 1 hour and rabbit anti-goat whole IgG alkaline phosphatase conjugate (1:10,000, Sigma) in 1% BSA/PBST for 45 min. Detection was finished by using CDP-Star (Roche, Indianapolis, Ind.) and Nitroblock Enhancer II (TROPIX, Bedford, Mass.) following manufacturers' protocols.

The process of anti-RTB western blotting was almost the same as for anti-mIL-12 western blotting except rabbit anti-RTB antibody (1:5000, Reidy and Cramer, unpublished) was applied to probe the membranes followed with goat anti-rabbit IgG conjugated with alkaline phosphatase (1:5000, Bio-Rad).

### Lactose Affinity Chromatography

About 2 ml of crude plant extracts were batch-purified with 100 μl lactose-conjugated agarose (CAT #CG004-5, EY Laboratories Inc., San Mateo, Calif.) overnight at 4° C. with gentle rocking. The mixtures were then loaded onto empty columns and centrifuged at 13,000 rpm. After washing once with 100 μl of PBS, proteins that bound to lactose resin were eluted by running 100 μl of PBS containing 0.25M galactose each time through the column twice.

### Southern Hybridization Analysis

Plant genomic DNA was extracted from leaf tissue by using Nucleon PHYTOPURE DNA extraction kit (Amersham) following manufacturer's protocols. Genomic DNA was digested by either HindIII or SacI, size-separated by 0.8% agrose gel electrophoresis and electroblotted onto hydrogen cellulose membrane (Amersham, Buckinghamshire, UK). In hybridization solution (260 mM sodium phosphate, 7% SDS, 1 mM EDTA, and 1% BSA, pH 7.2), membranes were probed using ˜800 bp fragments amplified from mIL-12 pBC plasmid and labeled with dCTP-³²P (PerkinElmer, Boston, Mass.) by using Prime-it RmT Random Primer Labeling Kit (Stratagene). After 2 days incubation at 65° C., the membrane was washed in hybridization wash solution (20 mM sodium phosphate, 1% SDS, 1 mM EDTA, 30 mM NaCl, pH 7.2) several times and exposed on film (XMR, CAT#1651496) for two days at −80° C.

### Development of Hairy Roots

Hairy roots were developed from selected transgenic tobacco lines expressing high levels of RTB-IL12 fusions as described previously. Liquid cultures were initiated with ˜20 root tips (1 cm) in a 250 ml flask containing B5 media (50 ml) and maintained under continuous light with shaking (90 rpm). About 1-week old hairy root cultures (50 ml media/250 ml flask) were transferred to PYREX® 2800 mL Fernbach-Style Culture Flasks (Item #4420-2XL, Corning) containing 0.5 L media and cultured for an additional 2 weeks. Tissues were then harvested and stored at −80° C.

Purification of Plant-Derived mIL-12:RTB

Hairy roots tissue was ground to a powder in liquid nitrogen and then homogenized in 2 volumes of grinding buffer (100 mM phosphate buffer, 20 mM galactose, pH7.6). Prior to chromatography, supernatants were diluted two-fold by adding an equal volume of deionized H₂O. The resulting mixture was filtered through a 0.45 μm membrane (Pall, Ann Arbor, Mich.) and subjected to cation exchange chromatography (Uno S column, Bio-Rad). Plant-derived mIL-12:RTB (IL-12:RTB) was eluted by a salt gradient from 0-1M NaCl in phosphate buffer. Fractions containing mIL-12 were pooled and applied to a lactose affinity column (CAT#CG004-5, EY Laboratories, self-packed). MIL-12:RTB was then eluted using 0.25M galactose in PBS. Fractions were examined by SDS-PAGE and visualized by silver staining. Eluates containing mIL-12:RTB were dialyzed against PBS, concentrated, quantified by mIL-12p70 ELISA (R&D Systems, Minneapolis, Minn.) and stored at 4° C. Visualization of silver-stained gels was used to estimate purity of IL-12:RTB.

### IL-12 Biological Activity Assay

IL-12 activity was determined by induction of IFN-γ in primary splenocytes from C57BL/6 mice and by stimulation of splenocyte proliferation.

### Culture of HT-29 Cell Monolayer

HT-29 cells (ATCC #HTB-38) were seeded onto cell culture inserts (CAT#PIHP01250, Millipore, Bedford, Mass.) at 7.5×10⁴/cm² and cultured in cell culture media (McCoy's 5a supplemented with 5 mM HEPES, 2 mM glutamine, 10% heat inactivated FBS, 100 U penicillin and 100 μg streptomycin; Gibco, Grand Island, N.Y.) for about 3-4 weeks, till cell monolayers formed tight conjunctions. To test the integrity of cell monolayer, the inserts were washed 3 times with 1% DMSO (Sigma) in Hank's balanced salt solution (HBSS, Gibco) and 200 μl of 60 μM of Lucifer yellow (Sigma) was added to the inserts. The inserts were placed in a 24-well plate (Greiner Bio-One, Monroe, N.C.) with 300 μl of 1% DMSO/HBSS in each well and cultured for 1 hour at 37° C./5% CO2. The concentration of Lucifer yellow in the bottom was measured by using a fluorescent plate reader (excitation 485 nm/emission 520 nm). Only when the concentration of Lucifer yellow was below 5 μM, the cell monolayer was considered to be tightly conjunct. Each insert has been checked by this integrity test before it was used for the transport assay.

### IL-12:RTB Activity Test in an In Vitro MALT Cell Culture Model

In order to assess the mucosal delivery potential of IL-12:RTB, plant-derived mIL-12 or mIL-12:RTB was added to the cell monolayer insert and the ability of IL-12 to stimulate IFN-γ production in splenocytes placed below the insert was determined. To initiate RTB-mediated transport assays, the HT-29 monolayer inserts were washed three times with cold Hank's balanced salt solution (HBSS) and then treated with 0.1% BSA/HBSS with 140 ng of plant-derived mIL-12:RTB or mIL-12 and incubated at 4° C. for 30 min. These inserts were then washed 3 times with warm cell culture media and incubated with 400 μl of cell culture media in each well. These inserts were then placed in a 24-well cell culture plates (Greiner Bio-One) with 600 μl of cell culture media in each well and incubated at 37° C. for 1 hour to permit endocytosis of bound RTB.

To provide mIL-12 responsive cells, mouse splenocytes were isolated from C57BL/6 mice (Jackson Laboratory) and placed in 24-well culture plates at 7.5×10⁵ cell/well in cell culture media (RPMI1640, 10% heat inactivated FBS, 5 mM HEPES, 2 mM glutamine, 100 U penicillin and 100 μg streptomycin) with 10 ng/ml of rhIL-2 (R&D Systems, Minneapolis, Minn.). The inserts that have been treated with IL-12:RTB or plant-derived IL-12 were put into these splenocytes wells and co-cultured overnight. The inserts were removed and the remaining splenocytes were cultured for an additional day. The supernatants from the 24-well plate were then collected and tested for IFN-γ ELISA kit (R&D Systems, Minneapolis, Minn.)

For RTB neutralization, 140 ng of mIL-12:RTB was mixed with RTB antibody (Reidy and Cramer, unpublished; 1:1000) before it was added to the inserts for cold treatment. The rest of the procedure was the same as described above.

### Results

Expression of RTB and mIL-12 Fusions in Transgenic Plants

For expression of RTB and mIL-12 fusions in transgenic plants, constructs that fused RTB to either the N-terminus (RTB:IL-12) or C-terminus (IL-12:RTB) of IL-12 were generated by utilizing RTB from plasmid R6-2 (Medina-Bolivar et al., 2003) and a single chain form of mIL-12 (Lieske et al., 1997). Constructs utilized a strong constitutive promoter de35S (the dual-enhanced 35S promoter, Lam et al., 1989), the tobacco etch virus (TEV) translational enhancer (Carrington et al., 1990), and sequences encoding the signal peptide provided either by the patatin signal peptide (pat, Iturriaga et al., 1989) upstream of RTB (FIG. 24), or the mIL-12 sequences (p40, endogenous) depending on orientation. These two constructs were then inserted into pBIB-Kan (Becker, 1990) for transformation of the plant cell via Agrobacterium tumefaciens-mediated transformation. Because the site of transgene insertion has significant impacts on expression in plants, we generated and screened 30-40 independent transformants for each construct. Crude leaf extracts of these transgenic plants were then screened by IL-12 conformational ELISAs which is correlated to the bioactivity of IL-12 and by asialofetuin-binding assays to assess RTB lectin activity. Asialofetuin contains 12 terminal galactose residues per molecules and exhibits high affinity for RTB, the carbohydrate-binding subunit of ricin (Dawson et al., 1995). As shown in Table 10, among RTB:IL-12 transgenic plants, less than a quarter of them showed detectable level of mIL-12 in crude extracts, and only one of those exhibited low level of functional RTB activity. In contrast, the majority of IL-12:RTB transgenic plants expressed functional mIL-12 and RTB, and the levels of mIL-12 were correlated to the levels of RTB in most plants. The best expressers of both constructs were utilized for further characterization.

TABLE 10
Screening of plants transformed by RTB:mIL-12 fusion constructs.

| | Number of plants | | Asialofetuin-binding |
|---|---|---|---|
| Constructs | screened | IL-12 ELISA (+)* | assay (+)* |
| RTB:IL-12 | 38 | 9 | 1 |
| IL-12:RTB | 34 | 21 | 22 |

*When the absorbance of screened plant is greater than that of non-transgenic control, it is positive.

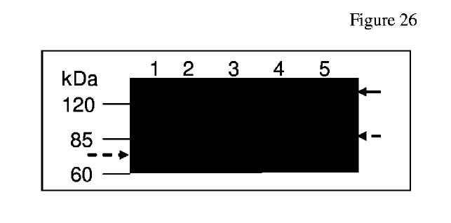

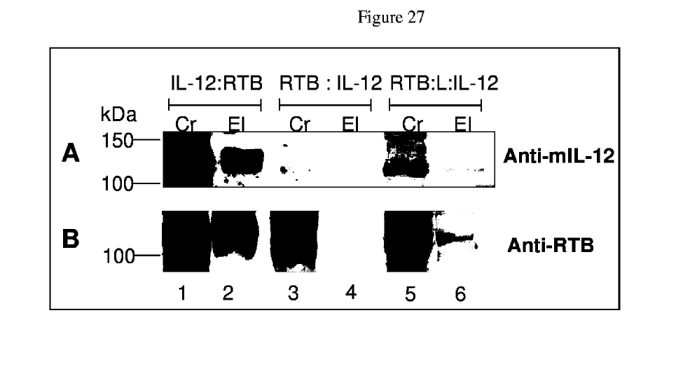

Crude leaf extracts from transgenic plants expressing fusion proteins were characterized by anti-mIL-12 western blotting analysis. FIG. 27 is a Western blot with the dashed arrow on the left showing mIL-12p70 and the arrow on the right indicating the breakdown products of the fusion proteins (around 70 kDa). Lane 1 is animal cell derived IL-12; lane 2, extracts from plants transformed by the IL-12:RTB construct; lane 3, extracts from plants expressing RTB: GFP fusion (non-IL-12 control; Medinar-Bolivar et al. 2003); lane 4, extracts from plants transformed by RTB:IL-12; and lane 5, extracts from plants transformed by RTB:L:IL-12 construct. Expression levels in plants producing IL-12:RTB were significantly higher than those producing RTB:IL-12 or RTB:L:IL-12. RTB:IL-12 plants showed a protein band of approximately ˜110 kDa that cross-reacted with anti-mIL-12 antibody (FIG. 26, lane 4), suggesting the production of a full length RTB:mIL-12 in transgenic plants. In addition to this ˜110 kDa band, there are other bands with faster mobility on SDS-PAGE that also cross-reacted with mIL-12 antibody. These bands are not present in non-mIL-12 transgenic extracts (FIG. 26, lane 3) or non-transgenic controls, suggesting they may represent breakdown products of full length RTB:mIL-12 (RTB:IL-12). The breakdown of RTB and mIL-12 has already been noted (Medina-Bolivar et al., 2003). IL-12:RTB transgenic plants produced a protein band with slightly higher molecular weight (˜120 kDa) than those from RTB:IL-12 plants (FIG. 26, lane 2 and 4), probably due to different post-translational modification or conformation. Similar to those from RTB:IL-12 plants, extracts from IL-12:RTB plants also demonstrated breakdown products of mIL-12:RTB (IL-12:RTB), which cross-reacted with mIL-12 antibody (FIG. 26, lane 2).

### IL-12:RTB, but not RTB:IL-12, Retains Full Lectin Activity

It has been shown that both IL-12:RTB and RTB:IL-12 transgenic plants produce full length fusion proteins. However, initial screening with the asialofetuin-binding assay suggested that the RTB:IL-12 arrangement resulted in products with reduced carbohydrate-binding activity. To explore this further, we utilized lactose-affinity chromatography, which is routinely used for affinity purification of RTB. In FIG. 27, lanes 1 and 2 are from IL-12:RTB expressing plants; lanes 3 and 4 from RTB:IL-12 expressing plants; and lanes 5 and 6 from RTB:L:IL-12 expressing plants. As shown in FIG. 27, the full length IL-12:RTB binds to lactose affinity column and can be eluted by high concentration of galactose (FIG. 27, lane 1 and 2), suggesting that RTB fused to the carbonyl-terminus of IL-12 retains lectin activity. In contract, the full length RTB:IL-12 was not recovered in the galactose eluate from lactose columns when the leaf extracts of RTB:IL-12 transgenic plants were applied to the column (FIG. 27, lane 3 and 4). This suggests that IL-12 fused to the carbonyl-terminus of RTB interferes with RTB lectin activity, probably due to steric hindrance and masking of active domains of RTB.

In order to determine whether the lack of lectin activity in RTB:IL-12 fusion is caused by steric hindrance, a polypeptide linker (Gly₃Ser)₃ was inserted in between RTB and mIL-12 in RTB:IL-12 construct. The resulting construct is called RTB:L:IL-12 and was utilized for plant transformation as described above. This 12-amino acid glycine-rich linker is very flexible in structure (Robinson et al., 1998) and may provide additional spacing to enable RTB and IL-12 to maintain functional three-dimensional structure. RTB:L:IL-12 plants produced a protein band slightly higher in molecular weight than the one from RTB:IL-12 plants (FIG. 26, lane 5), which is consistent with the presence of the polypeptide linker. The conformational IL-12 ELISA suggests that RTB:L:IL-12 maintains IL-12 bioactivity (data not shown). The full length RTB:L:IL-12 also binds to the lactose column (FIG. 27, lane 5 and 6), suggesting that the presence of polypeptide linker aids the fusion partners to retain their respective bioactivities.

### Characterization of Plant-Derived IL-12:RTB

Although RTB:L:IL-12 maintains IL-12 conformation and RTB lectin activity, its expression level in transgenic plants, like that of RTB:IL-12, is much lower than that of IL-12:RTB plants (FIG. 26, lane 2 and 5). As a result, IL-12:RTB plants were chosen for further characterization and in vitro biological activity assays. The top two expressers, IL-12:RTB plants IR6-11 and IR6-17 were analyzed by Southern hybridization to confirm transgene integration and copy number. Our result showed that each transgenic plant has 3 copies of transgene integrated (data not shown). Hairy roots, a rapid biomass accumulating culture system, were developed from these top two expressers via Agrobacterium rhizogenes (ATCC#15834)-mediated transformation (Medina-Bolivar and Cramer, 2004). Around 50 g of hairy roots can be harvested from one liter of media after 21-day culture.

Different grinding buffers were tested for maximum extraction of the transgenic product from hairy roots. A simple buffer, 20 mM galactose in 100 mM sodium phosphate buffer (pH 7.6), recovered the most transgenic products from the plant tissue. Galactose was introduced into the grinding buffer based on the rationale that galactose can compete for the galactose-binding domains on RTB, thus enhancing IL-12:RTB release from the glycan-rich plant tissue.

In order to effectively assess RTB-mediated uptake and delivery of functional IL-12, it was necessary to purify the product. It has already been shown that mIL-12 can be purified from plant extracts by cation-exchange column previously while RTB can be affinity-purified using lactose column (FIG. 27). Therefore, a two-step protein purification scheme combining cation-exchange and lactose affinity chromatography was developed for plant-derived IL-12:RTB purification. This purification scheme recovers about 30% of IL-12:RTB from the crude extraction and typically yields about 1 μg of purified protein per gram of fresh weight of hairy roots. All quantification of purified IL-12:RTB was based on IL-12p70 conformational ELISA.

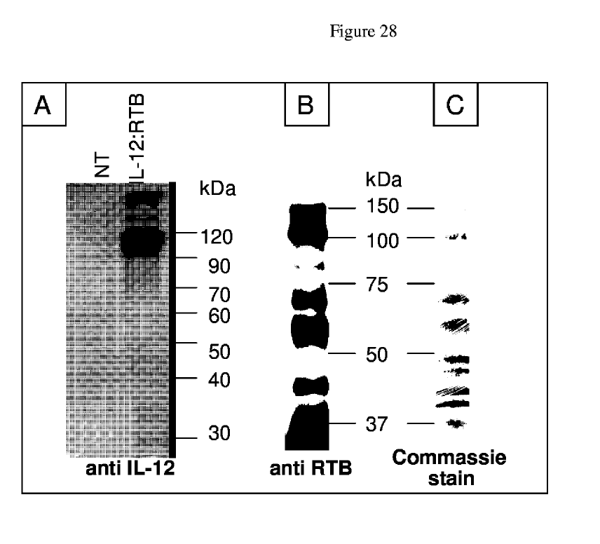

The purified plant-derived IL-12:RTB showed above 90% purity on silver-stained SDS-PAGE gel under non-reducing condition (FIG. 28A). However, under reducing condition, beside the full length protein bands, there are also many smaller size bands ranged from ˜30 kDa to ˜90 kDa (FIG. 28B). At least four of these breakdown bands cross-reacted with anti-RTB antibody (FIG. 28C). These results suggest that some peptide bonds in plant-derived IL-12:RTB probably have been cleaved by plant proteases. However, these breakdown products still assemble together through disulfide bonds and maintain the biological functions. The potential proteolytic sites may be identified by sequencing the N-terminus of these breakdown protein bands.

### Plant-Derived IL-12:RTB Exhibits IL-12 Biological Activity In Vitro

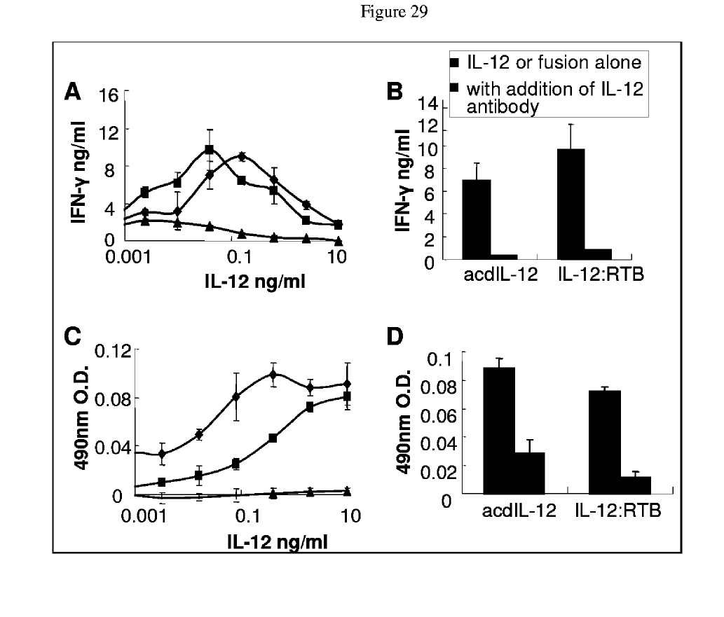

To confirm plant-derived IL-12:RTB maintains full IL-12 bioactivity, this purified recombinant fusion protein was studied for in vitro bioactivity in cultured splenocytes isolated from C57BL/6 mice. FIG. 29 graphs IL-12 bioactivity assay in mouse splenocytes. In FIG. 29A splenocytes from C57B/6 mice were cultured for 48 hours, with 10 ng/ml rhIL-2 and the indicated amounts of animal cell-derived mIL-12 (acdIL-12), IL-12:RTB purified from transgenic hairy roots (IL-12:RTB), or equivalent fractions from non-transgenic hairy root controls (NT). Supernatants were assayed for IFN-γ concentration by ELISA. In FIG. 29B induction of IFN-γ in the presence or absence of IL-12 neutralizing antibody is shown. IL-12 from different sources was incubated with or without IL-12 neutralizing antibody for two hours prior to the addition to mouse splenocyte cultures. Data from 0.04 ng/ml of IL-12 is shown. In FIG. 29C, standard colorimetric cell proliferation assays were performed on splenocytes as described in the material and methods. PHA-preactivated splenocytes were cultured for 48 hours with 10 ng/ml rhIL-12 and the indicated amounts of acdIL-12, IL-12:RTB or NT control. In FIG. 29D comparison of cell proliferation in splenocytes treated with IL-12 in the presence or absence of neutralizing antibodies. The data is representative of three separate experiments.

The results showed plant-derived IL-12:RTB demonstrated a dose-dependent activity in inducing the secretion of IFN-γ from splenocytes and stimulation of the proliferation of PHA pre-activated splenocytes, similar to the bioactivity of plant-derived mIL-12. These activities were blocked by pre-incubating IL-12:RTB with mIL-12 neutralizing antibody, suggesting they represent mIL-12 specific activities (FIGS. 29 B and D). From these assays, we also found that RTB alone did not stimulate IFN-γ secretion. RTB neutralization with anti-RTB antibody did not block IL-12 activity.

RTB Facilitate the Uptake of its Fusion Partner mIL-12

Our results showed that plant-derived IL-12:RTB exhibited IL-12 bioactivity and functional RTB activity. The purpose of producing IL-12:RTB fusion was to test whether RTB can function as a molecular carrier to facilitate the mucosal delivery of IL-12. Therefore, an in vitro transport assay model was modified to imitate the mucosal-associated lymphoid tissue (MALT). The monolayer of HT-29 cells (a human intestinal epithelial cell line) has been routinely utilized as an in vitro model of the intestinal epithelium to study drug transport and metabolism (Behrens et al., 2001; Blais et al., 1997; Thomson et al., 1997; Walter et al., 1996). We modified this intestinal epithelium model by adding splenocytes as immune responsive cells to the basolateral side of monolayer. We reasoned that because transcytosis is among the routes of ricin trafficking in mammalian cells, RTB may mediate epithelial cell uptake of IL-12 and delivery of the fusion product across the HT-29 monolayer to stimulate the IL-12 responsive splenocytes in the well below to produce IFN-γ. We use plant-derived mIL-12 as the control for “leakage”, i.e. splenocyte stimulation caused by limited amount of IL-12 that moves between cells of the monolayer.

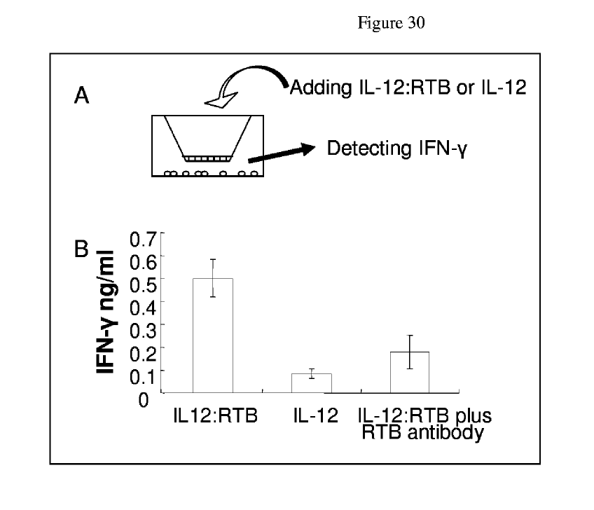

Plant-derived IL-12:RTB and controls were applied to the apical side of the cell monolayer to investigate whether IL-12 can be transported to the basolateral side and activate splenocytes. In FIG. 30, results are summarized. FIG. 30A shows a schematic of testing IL-12:RTB in MALT in vitro model. FIG. 30B shows results from such testing. About 140 ng of purified IL-12:RTB or IL-12 from transgenic plants was added to inserts containing a HT-29 monolayer and incubated at 4° C. for thirty minutes to allow RTB to bind to the cell surface. After being washed three times, the monolayer of cells was then co-cultured with splenocytes from C57BL/6 mice. After culturing for 2 days, supernatants of splenocytes were collected for IFN-γ ELISA. For RTB neutralization, RTB antibody (1:1000) was added to 140 ng of IL-12:RTB prior to incubation with monolayer of cells at 4° C. The data are representative to two separate experiments. Our results showed that IL-12:RTB stimulated the splenocytes to secrete IFN-γ (FIG. 30), suggesting that RTB facilitates the delivery of its covalently linked fusion partner IL-12 from the apical to the basolateral surface of monolayer where IL-12 was bioactive in stimulating splenocytes. In contrast, much less of IFN-γ was produced from splenocytes when co-culturing with HT-29 cell monolayer that has been treated with equivalent amount of plant-derived IL-12 (FIG. 30).

IL-12:RTB, co-incubated with anti-RTB polyclonal antibody also showed reduced potential to stimulated IFN-γ production in the splenocytes below suggesting the RTB activity was vital in mediating delivery of IL-12 across the HT-29 cell monolayer. Our results strongly support that RTB functions as a molecular carrier and facilitates the delivery of IL-12 across the mucosal epithelium to immune responsive cells in this model.

Discussion

IL-12 shows great promise as an anti-tumor therapeutic and a viral and cancer vaccine adjuvant (reviewed by Colombo and Trinchieri, 2002). The clinical application of IL-12 has been hindered by its toxicity associated with systemic administration (Leonard et al., 1997). However, it has been reported that localized delivery of IL-12 is effective and less toxic (reviewed by Salem et al., 2006). We have shown that RTB, the non-toxic glycan-binding subunit of ricin, may function as a molecular carrier to facilitate the mucosal delivery of IL-12. Previous studies have shown that RTB and mIL-12 can be successfully produced in transgenic plants, respectively (Medinar-Bolivar et al., 2003; Reed et al., 2005). In this example, mIL-12:RTB (IL-12:RTB) fusions were produced in transgenic plants and the bioactivity of both fusion partners was demonstrated. An effective purification scheme was developed for plant-derived IL-12:RTB yielding approximately 1 μg of purified protein per gram of fresh weight of hairy roots. The purified fusion protein stimulated production of IFN-γ in mouse splenocytes (mIL-12 bioactivity) and showed high binding affinity to the glycan-rich cell surface (RTB bioactivity). An in vitro model of MALT was utilized to demonstrate that RTB facilitates the delivery of mIL-12 through a cultured cell monolayer to immune responsive cells.

It is generally believed that orientation of fusion partners is critical to produce functional fusion proteins (Wittenmayer et al., 2000). Our results suggest that RTB fused to the amino-terminus of mIL-12 did not show full lectin activity. The insertion of a polypeptide glycine-serine linker (Gly₃Ser)₃ between RTB and mIL-12 resulted in an RTB-active lectin product that bound to lactose columns (FIG. 27). The glycine-serine linker is very flexible in structure (Robinson et al., 1998), which can provide extra spacing for fusion partners to fold correctly without interference.

Plant-derived IL-12:RTB demonstrated full bioactivity and high expression level in transgenic plants. It should be a single-chain fusion protein based on the construct which has been utilized to generate transgenic plants (FIG. 24). Under non-reducing condition, plant-derived IL-12:RTB showed doublet bands at around 120 kDa on silver-stained SDS-PAGE gel (FIG. 28), probably due to different glycosylation. However, multiple breakdown bands were observed on reducing SDS-PAGE gel and four bands cross-reacted with RTB antibody. This breakdown of single-chain protein in plant production system was not a separated incident. The polypeptide linker (Gly₄Ser)₃ that combines the two subunits of mIL-12 together in this single-chain construct, has been shown to be susceptible to some plant proteases. Moreover, when RTB is fused to the carbonyl-terminus of its fusion partner, the amino-terminus of RTB has been shown to be susceptible to plant proteases. Edman sequencing of these breakdown bands may help to identify the potential proteolytic sites in transgenic plants. Nevertheless, these cleavages of certain peptide bonds in plant-derived recombinant proteins do not affect their bioactivities. Plant-derived mIL-12 and IL-12:RTB exhibited full bioactivities, and are equivalent to animal-cell-derived mIL-12.

For mucosal delivery of protein, if the protein does not bind to the mucosal tissue, most of it would be washed away by body fluids circulating around all the time. RTB binds to the glycan-rich mucosal tissue so that it may work as a molecular carrier and facilitate the delivery of the fusion partner to mucosal functional sites. Our results indicate that RTB facilitated the delivery of its fusion partner IL-12 through the epithelial monolayer and as result stimulated the immune responsive cells. Because RTB is involved in multiple transport pathways inside the cell (reviewed by Sandvig et al., 2000), it aids in targeting its fusion partner to some specific organelles, and assists the presentation of its fusion partner to different type of cells in vivo.

### REFERENCES

Becker D. Binary vectors which allow the exchange of plant selectable markers and reporter genes. Nucleic Acids Res. 1990; 18: 203.
Behrens I, Stenberg P, Artursson P, Kissel T. Transport of lipophilic drug molecules in a new mucus-secreting cell culture model based on HT29-MTX cells. Pharm. Res. 2001; 18: 1138-1145.
Blais A, Aymard P, Lacour B. Paracellular calcium transport across Caco-2 and HT29 cell monolayers. Pflugers Arch. 1997; 434: 300-305.
Carrington J C, Freed D D. Cap-independent enhancement of translation by a plant potyvirus 5′ nontranslated region. J. Virol. 1990; 64: 1590-1597.
Chen H, Nelson R S, Sherwood J L. Enhanced recovery of transformants of Agrobacterium tumefaciens after freeze-thaw transformation and drug selection. Biotechniques. 1994; 16:664-8, 670.
Colombo M P, Trinchieri G. Interleukin-12 in anti-tumor immunity and immunotherapy. Cytokine Growth Factor Rev. 2002; 13: 155-168.
Dawson R M, Paddle B M, Alderton M R. Characterization of the Asialofetuin microtitre plate-binding assay for evaluating inhibitors of ricin lectin activity. J. Appl. Toxicol. 1999; 19: 307-312.
Endo Y, Tsurugi K. RNA N-glycosidase activity of ricin A-chain. Mechanism of action of the toxic lectin ricin on eukaryotic ribosomes. J. Biol. Chem. 1987; 262: 8128-8130.
Frankel A E, Fu T, Burbage C et al. Lectin-deficient ricin toxin intoxicates cells bearing the D-mannose receptor. Carbohydr. Res. 1997; 300: 251-258.
Gutierrez-Ortega A, Avila-Moreno F, Saucedo-Arias L J, Sanchez-Tones C, Gomez-Lim M A. Expression of a single-chain human interleukin-12 gene in transgenic tobacco plants and functional studies. Biotechnol. Bioeng. 2004; 85: 734-740.
Gutierrez-Ortega A, Sandoval-Montes C, Olivera-Flores T J, Santos-Argumedo L, Gomez-Lim M A. Expression of functional interleukin-12 from mouse in transgenic tomato plants. Transgenic Res. 2005; 14: 877-885.
Huber V C, Arulanandam B P, Amaboldi P M et al. Delivery of IL-12 intranasally leads to reduced IL-12-mediated toxicity. Int. Immunopharmacol. 2003; 3: 801-809.
Iturriaga G, Jefferson R A, Bevan M W. Endoplasmic reticulum targeting and glycosylation of hybrid proteins in transgenic tobacco. Plant Cell 1989; 1: 381-390.
Kwon T H, Seo J E, Kim J, Lee J H, Jang Y S, Yang M S. Expression and secretion of the heterodimeric protein interleukin-12 in plant cell suspension culture. Biotechnol. Bioeng. 2003; 81: 870-875.
Lam E, Benfey P N, Gilmartin P M, Fang R X, Chua N H. Site-specific mutations alter in vitro factor binding and change promoter expression pattern in transgenic plants. Proc. Natl. Acad. Sci. U.S.A 1989; 86: 7890-7894.
Leonard J P, Sherman M L, Fisher G L et al. Effects of single-dose interleukin-12 exposure on interleukin-12-associated toxicity and interferon-gamma production. Blood 1997; 90: 2541-2548.
Lieschke G J, Rao P K, Gately M K, Mulligan R C. Bioactive murine and human interleukin-12 fusion proteins which retain antitumor activity in vivo. Nat. Biotechnol. 1997; 15: 35-40.
Magnusson S, Kjeken R, Berg T. Characterization of two distinct pathways of endocytosis of ricin by rat liver endothelial cells. Exp. Cell Res. 1993; 205: 118-125.
Medina-Bolivar F, Wright R, Funk V et al. A non-toxic lectin for antigen delivery of plant-based mucosal vaccines. Vaccine 2003; 21: 997-1005.
Medina-Bolivar F, Cramer C. Production of recombinant proteins by hairy roots cultured in plastic sleeve bioreactors. Methods Mol. Biol. 2004; 267: 351-363.
Mohanraj D, Ramakrishnan S. Cytotoxic effects of ricin without an interchain disulfide bond: genetic modification and chemical crosslinking studies. Biochim Biophys. Acta 1995; 1243: 399-406.
Newton D L, Wales R, Richardson P T et al. Cell surface and intracellular functions for ricin galactose binding. J. Biol. Chem. 1992; 267: 11917-11922.
Reed D G, Nopo-Olazabal L H, Funk V et al. Expression of functional hexahistidine-tagged ricin B in tobacco. Plant Cell Rep. 2005; 24: 15-24.
Roberts L M, Smith D C. Ricin: the endoplasmic reticulum connection. Toxicon 2004; 44: 469-472.
Robinson C R, Sauer R T. Optimizing the stability of single-chain proteins by linker length and composition mutagenesis. Proc. Natl. Acad. Sci.U.S.A 1998; 95: 5929-5934.
Salem M L, Kadima A N, Zhou Y et al. Paracrine release of IL-12 stimulates IFN-gamma production and dramatically enhances the antigen-specific T cell response after vaccination with a novel peptide-based cancer vaccine. J. Immunol. 2004; 172: 5159-5167.
Salem M L, Gillanders W E, Kadima A N et al. Review: novel nonviral delivery approaches for interleukin-12 protein and gene systems: curbing toxicity and enhancing adjuvant activity. J. Interferon Cytokine Res. 2006; 26: 593-608.
Sandvig K, van Deurs B. Entry of ricin and Shiga toxin into cells: molecular mechanisms and medical perspectives. EMBO J. 2000; 19: 5943-5950.
Smith D C, Gallimore A, Jones E et al. Exogenous peptides delivered by ricin require processing by signal peptidase for transporter associated with antigen processing-independent MHC class I-restricted presentation. J. Immunol. 2002; 169: 99-107.
Thomson A B, Doring K, Keelan M, Armstrong G. Nutrient uptake into undifferentiated and differentiated HT-29 cells in culture. Can. J. Physiol Pharmacol. 1997; 75: 351-356.
Trinchieri G. Interleukin-12: a cytokine produced by antigen-presenting cells with immunoregulatory functions in the generation of T-helper cells type 1 and cytotoxic lymphocytes. Blood 1994; 84: 4008-4027.
Trinchieri G. Interleukin-12 and the regulation of innate resistance and adaptive immunity. Nat. Rev. Immunol. 2003; 3: 133-146.
van Deurs B, Hansen S H, Petersen O W, Melby E L, Sandvig K. Endocytosis, intracellular transport and transcytosis of the toxic protein ricin by a polarized epithelium. Eur. J. Cell Biol. 1990; 51: 96-109.
Walter E, Janich S, Roessler B J, Hilfinger J M, Amidon G L. HT29-MTX/Caco-2 cocultures as an in vitro model for the intestinal epithelium: in vitro-in vivo correlation with permeability data from rats and humans. J. Pharm. Sci. 1996; 85: 1070-1076.
Wittenmayer N, Rothkegel M, Jockusch B M, Schluter K. Functional characterization of green fluorescent protein-profilin fusion proteins. Eur. J. Biochem. 2000; 267: 5247-5256.

What is claimed is:

1. A method of preparing a molecule of interest for delivery to a eukaryotic cell, cell compartment, or combination of cells, the method comprising
(a) producing a recombinant ricin B chain subunit, said subunit comprising a recombinant ricin B chain subunit which does not have a ribosome inactivating subunit and which ricin B chain subunit retains lectin activity, wherein said recombinant B chain subunit is modified by a modification selected from the group consisting of
(i) modification of the first cysteine residue of said recombinant ricin B chain subunit such that the residue is not present or comprises an amino acid other than cysteine;
(ii) modification of amino acids at the N-terminal region of the subunit comprising a protease sensitive site such that the activity of the protease sensitive site is eliminated; and
(iii) addition of amino acids comprising a mammalian endoplasmic reticulum retrieval signal; and
(b) operatively associating the recombinant ricin B chain subunit with a molecule of interest.

2. The method of claim 1, wherein the protease sensitive site comprises the amino acids A, D, V, C/S, M and D and the site is eliminated by removing or substituting a different amino acid for the amino acids.

3. The method of claim 1, wherein the recombinant ricin B chain subunit is operatively associated with the molecule of interest by a process selected from the group consisting of conjugation, covalent binding, protein-protein interactions and genetic fusion.

4. The method of claim 1, wherein the recombinant ricin B chain subunit comprises primary amines and the recombinant ricin B chain subunit and the molecule of interest are operatively associated by chemical conjugation at least one of the primary amines of the recombinant ricin B subunit.

5. The method of claim 1, wherein the recombinant ricin B subunit comprises glycans and the subunit and the molecule of interest are operatively associated by chemical conjugation with at least one of the N-linked glycans of ricin B.

6. The method of claim 1, wherein biotin is chemically conjugated to ricin B and the molecule of interest is operatively associated with strepavidin such that the streptavidin binds to biotin.

7. The method of claim 1, wherein the ricin B subunit is operatively associated with the molecule of interest by a disulfide bond.

8. The method of claim 1, wherein the recombinant ricin B subunit is genetically fused to the molecule of interest such that the molecule of interest is linked at a site consisting of the N-terminus of the subunit, the C-terminus of the subunit and both the N-terminus of the subunit and C-terminus of the subunit.

9. The method of claim 8, further comprising providing a linker between the molecule of interest and the subunit.

10. The method of claim 9, wherein the linker is cleavable.

11. The method of claim 1, wherein the endoplasmic reticulum retrieval signal is selected from the group consisting of KDEL, HDEL and SEKDEL.

12. The method of claim 1, wherein the molecule of interest is linked at the C-terminus of the recombinant ricin B chain subunit and the endoplasmic reticulum retrieval signal is linked to the molecule of interest.

13. The method of claim 1, wherein the molecule of interest is linked at the N-terminus of the subunit and the endoplasmic reticulum retrieval signal is linked to the C-terminus of the ricin B subunit.

14. The method of claim 1, wherein at least one first immunoglobulin domain is fused to the recombinant ricin B chain subunit, providing at least one second immunoglobulin domain such that said first and second immunoglobulin domain assemble.

15. The method of claim 14, wherein the at least one first immunoglobulin domain and the at least one second immunoglobulin domain assemble to form the molecule of interest.

16. The method of claim 14, wherein the molecule of interest comprises the at least one second immunoglobulin domain, and assembly of the at least one first and the at least one second immunoglobulin domains operatively associate the molecule of interest with the subunit.

17. The method of claim 14, wherein the at least one second immunoglobulin is fused to the molecule of interest, and assembly of the at least one first and at least one second immunoglobulin domains operatively associate the molecule of interest with the subunit.

18. The method of claim 14, wherein the at least one first immunoglobulin domain and at least one second immunoglobulin domain is selected from the group consisting of a light chain and a Fd portion of a heavy chain of immunoglobulin.

19. The method of claim 14, wherein assembly of the at least one first immunoglobulin domain and at least one second immunoglobulin domain form a disulfide bond.

20. The method of claim 1, wherein the molecule of interest and the recombinant ricin B subunit are prepared in a host cell selected from the group consisting of a plant cell, an insect cell, a mammalian cell and a fungal cell.

21. The method of claim 1, wherein the molecule of interest and the recombinant ricin B subunit is prepared in a plant cell.

22. The method of claim 21, wherein the recombinant ricin B subunit is expressed at levels of at least about 0.1% total soluble protein.

23. The method of claim 1, wherein more than one molecule of interest is operatively associated with the recombinant ricin B subunit.

24. The method of claim 23, wherein at least two different molecules of interest are operatively associated with the recombinant ricin B subunit.

25. A method of delivery of a molecule of interest to a eukaryotic cell, cell compartment, or combination of cells, the method comprising preparing a molecule of interest according to the method of claim 1 and delivering the recombinant ricin B subunit operatively associated with the molecule of interest to the cell, cell component or combination of cells.

26. The method of claim 25, wherein the recombinant ricin B subunit is operatively associated with the molecule of interest by a disulfide bond and further comprising breaking the disulfide bond after delivery to the cell, cell compartment or combination of cells.

27. The method of claim 25, wherein the molecule of interest is delivered to a cell, cell compartment or combination of cells selected from the group consisting of a cell, a protoplast, immune cell, a serum cell, a macrophage, an animal cell, an epithelial cell, tissue of an animal, an organ of an animal, and mucosal tissue.

28. The method of claim 25, wherein the molecule of interest is delivered into a cell and further comprising delivery to a subcellular location by a process selected from the group consisting of delivery to cell lysosomes, delivery to cell endoplasmic reticulum, delivery to cell nucleus and delivery to the cytosolic compartment of the cell.

29. The method of claim 25, wherein the delivery to the cell, cell compartment or combination of cells is by a process selected from the group consisting of delivery across a cell layer by transcytosis, delivery to mucosa, and delivery across mucosa.

30. The method of claim 1, further comprising administering the recombinant ricin B chain subunit and operably associated molecule of interest to an animal by a method selected from the group consisting of nasal, oral, buccal, transdermal, dermal, inhalational, colonic, anal, vaginal, intravenous, intramuscular, intraperitoneal and subcutaneous administration.

31. A cell of a plant wherein the plant expresses recombinant ricin B chain subunit at levels of at least about 0.1% total soluble protein.


SEQUENCE LISTING (59 sequences)

SEQ ID NO: 1 - DNA, 789 bp, Native Ricinus communis
gctgatgtttgtatggatcctgagcccatagtgcgtatcgtaggtcgaaatggtctatgtgttgatgttagggatggaagattccacaacggaaacgcaatacagttgtggccatgcaagtctaatacagatgcaaatcagctctggactttgaaaagagacaatactattcgatctaatggaaagtgtttaactacttacgggtacagtccgggagtctatgtgatgatctatgattgcaatactgctgcaactgatgccacccgctggcaaatatgggataatggaaccatcataaatcccagatctagtctagttttagcagcgacatcagggaacagtggtaccacacttacggtgcaaaccaacatttatgccgttagtcaaggttggcttcctactaataatacacaaccttttgttacaaccattgttgggctatatggtctgtgcttgcaagcaaatagtggacaagtatggatagaggactgtagcagtgaaaaggctgaacaacagtgggctctttatgcagatggttcaatacgtcctcagcaaaaccgagataattgccttacaagtgattctaatatacgggaaacagttgttaagatcctctcttgtggccctgcatcctctggccaacgatggatgttcaagaatgatggaaccattttaaatttgtatagtggattggtgttagatgtgaggcgatcggatccgagccttaaacaaatcattctttaccctctccatggtgacccaaaccaaatatggttaccattattttga

SEQ ID NO: 2 - protein, 262 aa, Native Ricinus communis
ADVCMDPEPIVRIVGRNGLCVDVRDGRFHNGNAIQLWPCKSNTDANQLWTLKRDNTIRSNGKCLTTYGYSPGVYVMIYDCNTAATDATRWQIWDNGTIINPRSSLVLAATSGNSGTTLTVQTNIYAVSQGWLPTNNTQPFVTTIVGLYGLCLQANSGQVWIEDCSSEKAEQQWALYADGSIRPQQNRDNCLTSDSNIRETVVKILSCGPASSGQRWMFKNDGTILNLYSGLVLDVRASDPSLKQIILYPLHGDPNQIWLPLF

SEQ ID NO: 3 - protein, 262 aa, Native Ricinus communis
ADVCMDPEPIVRIVGRNGLCVDVRDGRFHNGNAIQLWPCKSNTDANQLWTLKRDNTIRSNGKCLTTYGYSPGVYVMIYDCNTAATDATRWQIWDNGTIINPRSSLVLAATSGNSGTTLTVQTNIYAVSQGWLPTNNTQPFVTTIVGLYGLCLQANSGQVWIEDCSSEKAEQQWALYADGSIRPQQNRDNCLTSDSNIRETVVKILSCGPASSGQRWMFKNDGTILNLYSGLVLDVRRSDPSLKQIILYPLHGDPNQIWLPLF

SEQ ID NO: 4 - protein, 262 aa, Native Ricinus communis
ADVCMDPEPIVRIVGRNGLCVDVRDGRFHNGNAIQLWPCKSNTDANQLWTLKRDNTIRSNGKCLTTYGYPSGVYVMIYDCNTAATDATRWQIWDNGTIINPRSSLVLAATSGNSGTTLTVQTNIYAVSQGWLPTNNTQPFVTTIVGLYGLCLQANSGQVWIEDCSSEKAEQQWALYADGSIRPQQNRDNCLTSDSNIRETVVKILSCGPASSGQRWMFKNDGTILNLYSGLVLDVRASDPSLKQIILYPLHGDPNQIWLPLF

SEQ ID NO: 5 - protein, 262 aa, Native Ricinus communis
ADVCMDPEPIVRIVGRNGLCVDVRDGRFHNGNAIQLWPCKSNTDANQLWTLKRDNTIRSNGKCLTTYGYPSGVYVMIYDCNTAATDATRWQIWDNGTIINPRSSLVLAATSGNSGTTLTVQTNIYAVSQGWLPTNNTQPFVTTIVGLYGMCLQANSGKVWLEDCTSEKAEQQWALYADGSIRPQQNRDNCLTTDANIKGTVVKILSCGPASSGQRWMFKNDGTILNLYNGLVLDVRRSDPSLKQIIVHPVHGNLNQIWLPLF

SEQ ID NO: 6 - protein, 263 aa, Native Ricinus communis
ADVCMDPEPIVRIVGRNGLCVDVRDGRFHNGNAIQLWPCKSNTDANQLWTLKRDNTIRSNGKCLTTYGYSPGVYVMIYDCNTAATDATRWQIWDNGTIINPRSSLVLAATSGNSGTTLTVQTNIYAVSQGWLPTNNTQPFVTTIVGLYGMCLQANSGKVWLEDCTSEKAEQQWALYADGSIRPQQNRDNCLTTDANIKGTVVKILSCGPVSSGQRWMFKNDGTILNLYNGLVLDVRRSDPSSLKQIIVHPFHGNLNQIWLPLF

SEQ ID NO: 7 - protein, 262 aa, Ricinus communis
ADVCMDPEPIVRIVGRNGLCVDVTGEEFFDGNPIQLWPCKSNTDWNQLWTLRKDSTIRSNGKCLTISKSSPRQQVVIYNCSTATVGATRWQIWDNRTIINPRSGLVLAATSGNSGTKLTVQTNIYAVSQGWLPTNNTQPFVTTIVGLYGMCLQANSGKVWLEDCTSEKAEQQWALYADGSIRPQQNRDNCLTTDANIKGTVVKILSCGPASSGQRWMFKNDGTILNLYNGLVLDVRRSDPSLKQIIVHPFHGNLNQIWLPLF

SEQ ID NO: 8 - protein, 260 aa, Native Ricinus communis
ADVCMDPEPIVRIVGRNGLCVNVRDGRFNHGNAIQLWPCKSNTDANQLTLKRDNTIRSNGKCLTTYGYPSGVYVMIYDCNTAATTADREIWNNGTIINPRSSLVLAATSGNSGTTLTVQTNIYAVSQGPLFTNNTQPWVTTIVGLYGLCLQANSGQVVIEDSCSEKAEQQWALYASGNINPQQRRDNCLTSDSNIRETVVKILSCGPASSGERWMFKNDGTILNLYSGLVLDVRASDPSLKQIILYPLWGHDPNQLILPF

SEQ ID NO: 9 - DNA, 24 bp, Artificial sequence
  Synthetic primer sequence
tctagagctgatgtttctatggat

SEQ ID NO: 10 - DNA, 26 bp, Artificial sequence
  Synthetic primer sequence
gtcgactcaaaataatggtaaccata

SEQ ID NO: 11 - DNA, 20 bp, Artificial sequence
  Synthetic primer sequence
gatatacccatggtccagct

SEQ ID NO: 12 - DNA, 31 bp, Artificial sequence
  Synthetic primer sequence
ctgactgtcgacacagataccagggcaattc

SEQ ID NO: 13 - DNA, 28 bp, Artificial sequence
  Synthetic primer sequence
gtcgactcaacagataccagggcaattc

SEQ ID NO: 14 - DNA, 28 bp, Artificial sequence
  Synthetic primer sequence
gagctcgcagcaagattccaatatattg

SEQ ID NO: 15 - DNA, 26 bp, Artificial sequence
  Synthetic primer sequence
ctcgagtcaaaactgtgacgatggtg

SEQ ID NO: 16 - DNA, 26 bp, Artificial sequence
  Synthetic primer sequence
tctagaccagatcctagcgtaattac

SEQ ID NO: 17 - DNA, 29 bp, Artificial sequence
  Synthetic primer sequence
tctagaagtaaaggagaagaacttttcac

SEQ ID NO: 18 - DNA, 26 bp, Artificial sequence
  Synthetic primer sequence
gagctctttgtatagttcatccatgc

SEQ ID NO: 19 - DNA, 26 bp, Artificial sequence
  Synthetic primer sequence
gagctcccagatcctagcgtaattac

SEQ ID NO: 20 - DNA, 24 bp, Artificial sequence
  Synthetic primer sequence
ctttactcatctgcagagaacctc

SEQ ID NO: 21 - DNA, 25 bp, Artificial sequence
  Synthetic primer sequence
ctgcaggctgatgtttctatggatc

SEQ ID NO: 22 - DNA, 26 bp, Artificial sequence
  Synthetic primer sequence
ctgcaggatgctgcaccaactgtatc

SEQ ID NO: 23 - DNA, 29 bp, Artificial sequence
  Synthetic primer sequence
gtcgacctaacactcattcctgttgaagc

SEQ ID NO: 24 - DNA, 27 bp, Artificial sequence
  Synthetic primer sequence
tctagagatgctgcaccaactgtatcc

SEQ ID NO: 25 - DNA, 28 bp, Artificial sequence
  Synthetic primer sequence
ctcgagacactcattcctgttgaagctc

SEQ ID NO: 26 - DNA, 24 bp, Artificial sequence
  Synthetic primer sequence
ctgcagagagagcccaccatctac

SEQ ID NO: 27 - DNA, 25 bp, Artificial sequence
  Synthetic primer sequence
tctagaagagagcccaccatctacc

SEQ ID NO: 28 - DNA, 25 bp, Artificial sequence
  Synthetic primer sequence
ctcgagacagataccagggcaattc

SEQ ID NO: 29 - DNA, 24 bp, Artificial sequence
  Synthetic primer sequence
tctagagtccagctgctccagtct

SEQ ID NO: 30 - DNA, 24 bp, Artificial sequence
  Synthetic primer sequence
gaattcgtccagctgctccagtct

SEQ ID NO: 31 - DNA, 27 bp, Artificial sequence
  Synthetic primer sequence
gaattcgttgtgatgacccaaactcca

SEQ ID NO: 32 - DNA, 28 bp, Artificial sequence
  Synthetic primer sequence
gactcatatggtccagctgctccagtct

SEQ ID NO: 33 - DNA, 69 bp, Artificial sequence
  Synthetic primer sequence
ctgactaagcttgaagctggtggctgttgtcctggctgttgcggtggcggcaccggtctcgagctgact

SEQ ID NO: 34 - DNA, 69 bp, Artificial sequence
  Synthetic primer sequence
agtcagctcgagaccggtgccgccaccgcaacagccaggacaacagccaccagcttcaagcttagtcag

SEQ ID NO: 35 - DNA, 25 bp, Artificial sequence
  Synthetic primer sequence
gagtgtctcgagggtgatgtttcta

SEQ ID NO: 36 - DNA, 26 bp, Artificial sequence
  Synthetic primer sequence
ccatagaaacatcacctcgaacactc

SEQ ID NO: 37 - DNA, 28 bp, Artificial sequence
  Synthetic primer sequence
gagtgtctcgaggctggtgtttctatgg

SEQ ID NO: 38 - DNA, 28 bp, Artificial sequence
  Synthetic primer sequence
ccatagaaacaccagcctcgagacactc

SEQ ID NO: 39 - DNA, 26 bp, Artificial sequence
  Synthetic primer sequence
ctcgaggctgatggttctatggatcc

SEQ ID NO: 40 - DNA, 26 bp, Artificial sequence
  Synthetic primer sequence
ggatccatagaaccatcagcctcgag

SEQ ID NO: 41-46 - skipped (6 entries)

SEQ ID NO: 47 - protein, 10 aa, Artificial Sequence
  Ricin B
ADVCMDPEPI

SEQ ID NO: 48 - protein, 10 aa, Ebulin B from Sambucus Ebulus
GETCAIPAPF

SEQ ID NO: 49 - protein, 10 aa, Misletoe lectin from Viscum album
CSASEPTVRI

SEQ ID NO: 50 - protein, 10 aa, Abrin B Abrus precatorius
IVEKSKICSS

SEQ ID NO: 51 - DNA, 23 bp, Artificial sequence
  Synthetic primer sequence
gcactcgagaaataatggtaacc

SEQ ID NO: 52 - DNA, 27 bp, Artificial sequence
  Synthetic primer sequence
cgctctagagctgatgtttgtatggat

SEQ ID NO: 53 - DNA, 37 bp, Artificial sequence
  Synthetic primer sequence
cmgatcctcgagaaggatgagctttgagagctcgatc

SEQ ID NO: 54 - DNA, 35 bp, Artificial sequence
  Synthetic primer sequence
gatcgagctctcaaagctcatccttctcgaggatc

SEQ ID NO: 55 - DNA, 25 bp, Artificial sequence
  Synthetic primer sequence
ctcgagatgtgggagctggagaaag

SEQ ID NO: 56 - DNA, 25 bp, Artificial sequence
  Synthetic primer sequence
gagctctcaggcggaactcagatag

SEQ ID NO: 57 - DNA, 54 bp, Artificial sequence
  Synthetic primer sequence
acgctcgagggaggtggatcaggtggcggatctggtggaggttctctcgagtac

SEQ ID NO: 58 - DNA, 25 bp, Artificial sequence
  Synthetic primer sequence
ggtaccatgggtcctcagaagctaa

SEQ ID NO: 59 - DNA, 24 bp, Artificial sequence
  Synthetic primer sequence
gagctcggcggaactcagatagcc